SEQUENCE LISTING (60 sequences)

SEQ ID NO: 1 - DNA, 1362 bp, Oryza rufipogon
  misc_feature (638)..(699): n is a, c, g, or t
atgtgaaagctagacccgacgagctcgacattgctatcgttggtggaggcatggtgggattggctgttgcttgtgcattgtccaatatgccattgactaaacatctgagggttggcatcatcgatagtaatcctgcattgaagtcaagaaattatctgaagaaagatggtgtacctgattcgagggtcagtactattactcccgcgaccatatccttcttcagagatattggcgcatgggatcatgttcaacagcaaagacacgctttctttggtaaaatgcaggtatgggattacactggactgggttacacaaggtatagtgcaagagatgtgggaaaagaatatctcggatgtgtggtggagaacaaggtgctttgcaactcactgctgttacggttacaggaggaaaatggagacattgaaaagatgatctaccctactagactgatttctcttgctttaccatcaaagagcagacaagcaccaacaagggaagcaatatccagtggttaccctccagaagaattaaatcgcagcaatttagttaaacttgagctaagtgatggacaaactttatattcaaaactagtggtaggagctgatggttccaagtccaatgtaaggcaaacagcaggnnnnnnnnnnnnnnnnnnnnnnnnnnnnnnnnnnnnnnnnnnnnnnnnnnnnnnnnnnnnnnatttgtacagtggagcatattaccgaaaacgattgtgcatggcagaggtttctcccttctggtccaattgctctacttccaataggtgacaactacagcaatattgtgtggacaatgagcccagaggagtcattgcgccataagtcaatgagtcctgaagattttgtgaagtctgtgaacaatgcattggattttggttacggtccacatccacactctggtaatcttgactattacatggaaaagttattttctgacattggaagtactgctacatctacaaaggaatgctttgaggtacctccaaaagcaatcggggtagtctctgagagaatggcatttccattgtcgctgaagcattcacatgattatgtttcaaaaaggatagcactagtcggtgatgcagctcatacagtccatcccttagctggccaaggtgtcaacttaggttttggagatgcagctgctttagcaaaagttatcgctgatggagtttctgtgggtgctgattttggggatatttccatgctcaaaaggtatgagaatgatcgcaaagctgccaatgttgcaatggcagcgctgctagatggtttccagaagatgtactctgttgattttggtcccctcaatgtgcttagggctgcagcgtttcacggtgcccag

SEQ ID NO: 2 - DNA, 1312 bp, Oryza rufipogon
  misc_feature (588)..(649): n is a, c, g, or t
atggtgggattggctgttgcttgtgcattgtccaatatgccattgactaaacatctgagggttggcatcatcgatagtaatcctgcattgaagtcaagaaattatctgaagaaagatggtgtacctgattcgagggtcagtactattactcccgcgaccatatccttcttcagagatattggcgcatgggatcatgttcaacagcaaagacacgctttctttggtaaaatgcaggtatgggattacactggactgggttacacaaggtatagtgcaagagatgtgggaaaagaatatctcggatgtgtggtggagaacaaggtgctttgcaactcactgctgttacggttacaggaggaaaatggagacattgaaaagatgatctaccctactagactgatttctcttgctttaccatcaaagagcagacaagcaccaacaagggaagcaatatccagtggttaccctccagaagaattaaatcgcagcaatttagttaaacttgagctaagtgatggacaaactttatattcaaaactagtggtaggagctgatggttccaagtccaatgtaaggcaaacagcaggnnnnnnnnnnnnnnnnnnnnnnnnnnnnnnnnnnnnnnnnnnnnnnnnnnnnnnnnnnnnnnatttgtacagtggagcatattaccgaaaacgattgtgcatggcagaggtttctcccttctggtccaattgctctacttccaataggtgacaactacagcaatattgtgtggacaatgagcccagaggagtcattgcgccataagtcaatgagtcctgaagattttgtgaagtctgtgaacaatgcattggattttggttacggtccacatccacactctggtaatcttgactattacatggaaaagttattttctgacattggaagtactgctacatctacaaaggaatgctttgaggtacctccaaaagcaatcggggtagtctctgagagaatggcatttccattgtcgctgaagcattcacatgattatgtttcaaaaaggatagcactagtcggtgatgcagctcatacagtccatcccttagctggccaaggtgtcaacttaggttttggagatgcagctgctttagcaaaagttatcgctgatggagtttctgtgggtgctgattttggggatatttccatgctcaaaaggtatgagaatgatcgcaaagctgccaatgttgcaatggcagcgctgctagatggtttccagaagatgtactctgttgattttggtcccctcaatgtgcttagggctgcagcgtttcacggtgcccag

SEQ ID NO: 3 - DNA, 1536 bp, Oryza sativa
ctcgacattgctatcgttggtggaggcatggtgggattggctgttgcttgtgcattgtccaatatgccattgactaaacatctgagggttggcatcattgatagcaatcctgcattgaagtcaagaaattatctgaagaaagatggtgtacctgattcaagggtcagtactattactcccgcgaccatatccttcttcagagatattggtgcatgggatcatgttcaacagcaaagacacgctttctttggtaaaatgcaggtatgggattacactggactgggttacacaaggtatagtgcaagggatgtgggaaaagaatatctcggatgcgtggtggagaacaaggtgctttgcaactcactgttgttacgattacaggaggaaaatggagacattgaaaagatgatctaccctactagactgatttctcttgctttgccatcaaagagcagacaagcaccaacaagggaagcaatatccagtggttaccctccagaagaattaaatcgcagcaatttagttaaacttgagctaagtgatggacaaactttatattcaaaactagtggtgggagctgacggttccaagtccaatgtaaggcaaacagcaggcataaaaacaacagggtggaattatcctcaaagtgcaattatttgtacagtggagcatattaccgaaaacgattgtgcatggcagaggtttctcccttctgggccaattgctctacttccaataggtgacaactacagcaatattgtgtggacaatgagcccagaggagtcattgcgccataagtcaatgagtcctgaagattttgtgaagtctgtgaacaatgcattggattttggttacggtccacatccacactctggtagtcttgactattacatggaaaagttattttctgacattggaagtactgctgcatctacaaaggaatgctttgaagtacctccaaaagcaatcggggtagtctgtgagagaatggcatttccattgtcgctgaagcattcacatgattatatttcaaaaaggttagcactagtcggtgatgcagctcatacagtccatcccttagctggccaaggtgtcaacttaggttttggagatgcagctgctttagcaaaagttatcgctgaaggagtttctgtgggtgctgattttggggatatttccttgctcaaaaggtatgagaataatcgcaaagcagccaatgttgcaatggcagcgctgctagatggtttccagaagatgtactctgttgattttggacccctcaatgtgcttagggctgcagcgtttcacggtgcccagtacatatcaccactgaagaagaacataatctcgtatgcaatgggcgacgcgaaatggccgctgttttcatagacagtagatccggaaactttaaatttgttagttgttactgcagtgtcgatgtgttattctaaattagtatagtgtactttgtctccagtgttctcatccagagtagtaatgcatcacaggcacacgttgcagagaagaaatgtagct

SEQ ID NO: 4 - DNA, 1362 bp, Oryza sativa
atggtgggattggctgttgcttgtgcattgtccaatatgccattgactaaacatctgagggttggcatcattgatagcaatcctgcattgaagtcaagaaattatctgaagaaagatggtgtacctgattcaagggtcagtactattactcccgcgaccatatccttcttcagagatattggtgcatgggatcatgttcaacagcaaagacacgctttctttggtaaaatgcaggtatgggattacactggactgggttacacaaggtatagtgcaagggatgtgggaaaagaatatctcggatgcgtggtggagaacaaggtgctttgcaactcactgttgttacgattacaggaggaaaatggagacattgaaaagatgatctaccctactagactgatttctcttgctttgccatcaaagagcagacaagcaccaacaagggaagcaatatccagtggttaccctccagaagaattaaatcgcagcaatttagttaaacttgagctaagtgatggacaaactttatattcaaaactagtggtgggagctgacggttccaagtccaatgtaaggcaaacagcaggcataaaaacaacagggtggaattatcctcaaagtgcaattatttgtacagtggagcatattaccgaaaacgattgtgcatggcagaggtttctcccttctgggccaattgctctacttccaataggtgacaactacagcaatattgtgtggacaatgagcccagaggagtcattgcgccataagtcaatgagtcctgaagattttgtgaagtctgtgaacaatgcattggattttggttacggtccacatccacactctggtagtcttgactattacatggaaaagttattttctgacattggaagtactgctgcatctacaaaggaatgctttgaagtacctccaaaagcaatcggggtagtctgtgagagaatggcatttccattgtcgctgaagcattcacatgattatatttcaaaaaggttagcactagtcggtgatgcagctcatacagtccatcccttagctggccaaggtgtcaacttaggttttggagatgcagctgctttagcaaaagttatcgctgaaggagtttctgtgggtgctgattttggggatatttccttgctcaaaaggtatgagaataatcgcaaagcagccaatgttgcaatggcagcgctgctagatggtttccagaagatgtactctgttgattttggacccctcaatgtgcttagggctgcagcgtttcacggtgcccagtacatatcaccactgaagaagaacataatctcgtatgcaatgggcgacgcgaaatggccgctgttttcatag

SEQ ID NO: 5 - protein, 429 aa, Oryza sativa
MVGLAVACALSNMPLTKHLRVGIIDSNPALKSRNYLKKDGVPDSRVSTITPATISFFRDIGAWDHVQQQRHAFFGKMQVWDYTGLGYTRYSARDVGKEYLGCVVENKVLCNSLLLRLQEENGDIEKMIYPTRLISLALPSKSRQAPTREAISSGYPPEELNRSNLVKLELSDGQTLYSKLVVGADGSKSNVRQTAGIKTTGWNYPQSAIICTVEHITENDCAWQRFLPSGPIALLPIGDNYSNIVWTMSPEESLRHKSMSPEDFVKSVNNALDFGYGPHPHSGSLDYYMEKLFSDIGSTAASTKECFEVPPKAIGVVCERMAFPLSLKHSHDYISKRLALVGDAAHTVHPLAGQGVNLGFGDAAALAKVIAEGVSVGADFGDISLLKRYENNRKAANVAMAALLDGFQKMYSVDFGPLNVLRAAAFHGA

SEQ ID NO: 6 - DNA, 4649 bp, Oryza sativa
gatcacacagagaaggttgatgatgtgaaagctagacccgacgagctcgacattgctatcgttggtggaggcatggtgggattggctgttgcttgtgcattgtgtaggtattttctgttcatgttgtgcttccttgttttttgttggctctgcttactgtcagtcgttttcggctgtaaagaagacttctcccttattgttgtaggctatgatattgtctagaaaaaagtttttaacccagatgaaaatagttctcaacgatcctcaccgacctattagtgcaatttgaagtgctattaatgattcttttttttcattggtattggtatgagtagcttttcatgctatagttgaatcgcaccttagacatgtgaaaatgacgattggatgtttcaaatcagaaacacctgtcatttaaggatcaagtttttcacatgcttatttatccacatgcagccaatatgccattgactaaacatctgagggttggcatcattgatagcaatcctgcattgaagtcaagaaattatctgaagaaagatggtgtacctgattcaagggtcagtactattactcccgcgaccatatccttcttcagaggtacagtttttaatcgaacatgcgttgatattatctgtacgtgttcagaatttttcaggcgcttgtactttgaattaagtttttcttaacatggttgggtactttgttgatgacgttttatgttggttttgagcagatatataaacataatgctataatcgtttttgtattacttctatgtctccatcattttgtaaagttatctagttacatcattagtagtttattttattatcttcctttttactcttgtggatatctgcttcattatataaattagttgatagcctaccgcctacctgggatgatcactatgcaatattcggctgaactttctttcttttggagtacgtactacagtaccgtatagtgataagctgaagcgacttgtgaactccattgatctatatgctcaccatttccgttgcagtacagttgcatcatattatatatcctttctcatgtgtgatcttcacaattttttgctaaggtttcagccctatttttgcacattaaggctgggaaattgcatgcttgcacagtacagcttgagaattgctgtatctgtatttctcccttcaaacgtgcaaataagctacttttacctactagtgatggtagcgtaccattcaacaatttcttcatacttgtaccatgttatattgcagatattggtgcatgggatcatgttcaacagcaaagacacgctttctttggtaaaatgcaggtgatataccttgcttgtcctgatgaaatgatttgttctgtatgtatagtgaatatccttttattttcaatttcacctttactctatttttgtccaacacatttaactctatttcctctttgagcccacttatttctttaaagttcaaaattctgaacatgttgattcttattggactaggcggctttgttcttgcctttatatcaatatcaatgaaaaactaagtcagcctctgaattcatctactgcaggttccacatcaggctagcaataaaagatcattgtacttagaattttaaaatactcgtaatcagttctcaattggaacatagaatagttgtatacctatattatgcttctgtttgcatacccatatttttagtagtctggacccatacatgcaagtattagaacttggatcctttaccttttgtattttcttctttgatgttgaacttgcacaaaataagagcagtttcttcctccagatagtgggcgcttcagtttaagcactaactgcaattctggatgtgctcatgtgctatttgctaacaggtatgggattacactggactgggttacacaaggtatagtgcaagggatgtgggaaaagaatatctcgggtatgctctatgaaagcagctgtgatttatgactaatttctgtcaccaactcattactaacttcttatttattaattcaatgtttagatgcgtggtggagaacaaggtgctttgcaactcactgttgttacgattacaggtttgtcttcaattgacatatttgattttttttagtatctgcatggagcgtgatgtttgaactcgtctaataccactttaatctgtaaatggaattgtatgcttccttgcatatatttggaatagaacattcttaccatctatgcattcaagacagctaaatttatgaggcctttataaacacacacacacacacacacacaccattttatggttcctttcctcttggttaaccaatatagtttattagcagtaaaggttttgtgataataatagagtaaattgcattagcggtacatgaacttgtcaggtgggtgcactttagtgtatgaacttataaaatgctcgttttggtgcatgaacttgtttgatacatgcaaactaaggctaaaatgacacgacaagaccgatttttctatgctgaacattataaattataaattagtagatgatgtgcgtgcacatagtagaaatgttatatttgtaaacttactttccactttatctttctttgtttctatctcttacatcattactcggttttttattttatgcaatcgttatgcctatatgtttatccttcttagacacatgtttacatagagtttaaatcaacaagattaaccttgatgtgcgattttgaccttgttcgcacgcaccaaacaagtttgtgcaccaaaacaagcgttttacaagttcatgtactagattgcacccatctgacaagttcgtgtacctctgatacaatttactcataataatactcccacccatgtgtaggacagagtaaacctacatagaagttgatggtcgggttatttgcagccagataatcttacctttatttttagtaaggctaatgcttaaagatttttgcttgatcaggaggaaaatggagacattgaaaagatgatctaccctactagactgatttctcttgctttgccatcaaagagcagacaagcaccaacaagggaagcaatatccagtggttaccctccagaagaattaaatcgcagcaatttagttaaacttgagctaagtgatggacaaactttatattcaaaactagtggtaattgcctaccatcttcttttgcatttgttcaaacttctgtctttgtccattgccttcagttgcctacttaaatgtatggttgcaggtgggagctgacggttccaagtccaatgtaaggcaaacagcaggcataaaaacaacagggtggaattatcctcaaagtgcaattatttgtacagtggagcatattaccgaaaacgattgtgcatggcagaggtttctcccttctgggccaattgctctacttccaataggtgacaactacagcaatattgtgtggacaatgagcccagaggagtcattgcgccataagtcaatgagtcctgaagattttgtgaagtctgtgaacaatgcattggattttggttacggtccacatccacactctggtagtcttgactattacatggaaaagttattttctgacattggaagtactgctgcatctacaaaggaatgctttgaagtacctccaaaagcaatcggggtagtctgtgagagaatggcatttccattgtcgctgaagcattcacatgattatatttcaaaaaggttagcactagtcggtgatgcagctcatacagtccatcccttagctggccaaggtgtcaacttaggttttggagatgcagctgctttagcaaaagttatcgctgaaggagtttctgtgggtgctgattttggggatgtaagtacctcttgctgcaatcttttggttaacattttattcaaatactaacacaacctggattctattagcaaatatatcatcatcttgaaagttgaaacttaagtacttgactagatacaagtgaaagaaccgatcatgctgcatctgaaatattgtaattcatttcccctttagtgtagtgtaattcttgttggagtgcactataaacatcatatttatcatcacatcaaactaacatgataaatgattttatttgacagatttccttgctcaaaaggtatgagaataatcgcaaagcagccaatgttgcaatggcagcgctgctagatggtttccagaagatgtactctgttgattttggacccctcaatgtgcttagggctgcagcgtttcacggtgcccagtacatatcaccactgaagaagaacataatctcgtatgcaatgggcgacgcgaaatggccgctgttttcatagacagtagatccggaaactttaaatttgttagttgttactgcagtgtcgatgtgttattctaaattagtatagtgtactttgtctccagtgttctcatccagagtagtaatgcatcacaggcacacgttgcagagaagaaatgtagctagaaagaaataaattggatctcctggtgatctaggattaccttgaatgtactcctcttgcactccagtaactcgcttctgttttgacccacaaagttcagatctgtgcaaggcaagatgatattgaagtgttgactgttgacgaagatcagtcaatctgtgcaggttgca

SEQ ID NO: 7 - DNA, 26 bp, Oryza sativa
cacccatgtgtaggacagagtaaacc

SEQ ID NO: 8 - DNA, 32 bp, Oryza sativa
caacagagtacatcttctggaaaccatctagc

SEQ ID NO: 9 - DNA, 554 bp, Sorghum bicolor
  misc_feature (492)..(492): n is a, c, g, or t
caaccggtgtagtttttgagagaatggcatttccgttgtcattgatgcattcccatgattatgtttccaaaaggctagcattagttggagacgctgctcatactgtccaccccttagccggccaaggcgtcaacttgggctttggtgatgctgctgctttagcaaaagttattgctgaaggagtttctgtgggctcagatgttggggacctgactttgctacaacggtatgagaaggaccggaaggctgccaatgtggcgatgacagcagtgctagatggtttccagaagatgtactctgtggattttgggccccttaatgtagtgagagctgctgcgttccacggtgcccagtacatatcaccactgaaaaagaacatcatctcttatgcaatgggtgacaccaaatggccactatttcggtgaacagtactgctttgaaactgttgttctgaacttgtgcgatgcacctggcttatagtttcggcttgtnactggatcactaagaaacattgtagtgcagatgcaatattacagtcaatcaataaatttttt

SEQ ID NO: 10 - DNA, 823 bp, Saccharum officinarum
caactgggtggaattatccgcaaagtgcaattatctgtacagtagagcatgttgtggaaaatgattgtgcatggcagaggtttctcccttctggtccaattgcactacttccaataggcaacaacttcagcaacattgtatggacaatgagcccagaggagtcactgcgccataagtcaatgagccccgaagagcttgtgaagtcggtgaaccatgctttggattttggttatggtccacatcctcgctctactactcttgaccattacatggaacagttcttttctggcattgggaacactgcagcatctacaaaggaatgctttgaagtaccaccaaaagcaactggtgtagtttctgagagaatggcatttccattgtcattgatgcattcccgtgattatgtttccaaaaggctagcattagttggtgatgctgctcatactgtccaccccttagccggccaaggtgtcaacttgggctttggtgatgctgctgctttagcaaaagttattgctgaaggagtttctgtgggctcagatgttggggacgtaagtatctattgctgtttcttttagcctttcaacaacaacaacaacaacaacaacaacatagcctctttttgtcttgtgccctttccctctgaaatttaattttggaactatacagctgcacctgaaccattcttatgattcatggtgggtatgaattttagtggtgatgatattgccacttttgccagttctagatttctagttgaaccatacctaatactaattttaaacgttcttttttccctttggcaagctgactttgttaccac

SEQ ID NO: 11 - DNA, 778 bp, Triticum aestivum
  misc_feature (661)..(661): n is a, c, g, or t
tgatggttccaatccaatgtaaggcagattgcgggcataaaaactactggttggagctatcctcagagtgcgattatctgtacagtagagcatactgcagcgaatgactgtgcatggcagaggtttctcccttctggtccgattgccctgcttccaataggcgacaacttcagcaatatagtgtggacaatgagcccagaggaggcatcacgccataagtcaatgagccctgaagattttgtgatgtcagtgaaccgtgcattggattttggttatggaccgcatcccaactctagtagtcttgaccattacgtggaaaggttattttctgactttggaggtactgcagcgtctacaaaagaatgctttgaagtaccaccaagggcaactgggctgatctcagagagaatggcatttccattgtcactgatgcactcccatgattatgtttcaaaaaggctagcattagttggtgatgctgctcatacagtgcaccccttggccggtcaaggtgtcaatttgggttttggagacgcagctgccttagcggaagttatttctgaaggagtttctgttggtgctgatattggggatctgtctttgctgaatcggtatgagaatgaccgcaaggctgccaatgttgcgatggcggcagttctanatggcttccagaagatgtactctgtcgatttcggacccctcaatgttctgagagccgctgcattccatggtgcccagtacatatcaccactgaagaanaacataatttcttacncaa

SEQ ID NO: 12 - DNA, 600 bp, Triticum aestivum
gaattcggcacgagcataaaaactactggttggagctatcctcagagtgcgattatctgtacagtagagcatactgcagcgaatgactgtgcatggcagaggtttctcccttctggtccgattgccctgcttccaataggcgacaacttcagcaatatagtgtggacaatgagcccagaggaggcatcacgccataagtcaatgagccctgaagattttgtgatgtcagtgaaccgtgcattggattttggttatggaccgcatcccaactctagtagtcttgaccattacgtggaaaggttattttctgactttggaggtactgcagcgtctacaaaagaatgctttgaagtaccaccaagggcaactgggctgatctcagagagaatggcatttccattgtcactgatgcactcccatgattatgtttcaaaaaggctagcattagttggtgatgctgctcatacagtgcaccccttggccggtcaaggtgtcaatttgggttttggagacgcagctgccttagcggaagttatttctgaaggagtttctgttggtgctgatattggggatctgtctttgctgaatcggtatgagaat

SEQ ID NO: 13 - DNA, 626 bp, Triticum aestivum
gttggtggtaggagctgatggttccaaatccaatgtaaggcagattgcgggcataaaaacaactggttggagttatcctcagagtgccattatctgtacagtagagcatactgcagagaatgacactgcatggcagaggtttctcccttctggtccgattgccctgcttccaataggtgacaacttcagcaatatagtgtggacaatgagcccagaggaggcatcacgccataagtcaatgagctctgaagattttgtgatgtcagtgaaccgtgcattggattttggttatggaccccatcccaattctagtagccttgaccattacgtggaaaggttgttttctgactttggaggtactgcagcgtctacgaaagaatgctttgaagtaccaccaagggcaattgggctgatctcagagagaatggcatttccattgtccctgatgcactcccatgattatgtttcaaaaaggctagcattagttggtgatgctgctcatacagtgcaccccttggccggtcaaggtgtcaatttgggttttggagacgcagctgccttacaaaagttatttctgaaggagtttctgttggtgctgatattggggatctatctttgctgaaccg

SEQ ID NO: 14 - DNA, 563 bp, Hordeum vulgare
ggagttatcctcagagtgcgattatatgtacagtagagcatactgcaaagaatgattgtgcgtggcagaggtttctcccttctggtccaattgccctgcttccaataggtgacaacttcagcaatatagtgtggacaatgagcccagaggaggcatcacgccataagtcaatgagctctgaagattttgtgatggcagtgaaccgtgcattggattttggctatggaccgcatcccagctctactagtcttgaccattacatggaaaggttattttctggcgttggagacactgcagcgtctacaaaagaatgctttgaagtaccgccaagagcaatcgggttgatctctgagagaatggcatttccattgtcactgatgcattcccatgattatgtttcaaaaaggctagcactggttggtgatgctgctcatacagtccaccccttggccggtcaaggtgtcaatttgggttttggagatgcagctgccttagcggaagttatttctgaaggagtttctgttggtgctgatattggggatctatctttgctgaatcggtat

SEQ ID NO: 15 - DNA, 961 bp, Agrostis capillaries
ggatgaaggctgccaattcagctctagatggcttccagaagatgtgctctgttgatttcagacccctcaatgtcggtagagccgcagcattccatggttcccaatatcccattacatatcatcactgaagaagaacagctcttacgctatggttgacaggaagtctctatttcataggtaacaggtcttacacactccttggctgtttactgtcactgcactactatggtgattaaatgtgtagttttaaactagtatggtgtactcttgcttgtattgagtattctaagctagtgatgtttcgctcagaaacgctaaaaatagataaagtgtaataaagtaagattccctggcgtggttagaatattcattggatttattttcacctaggtgatctcaatgttcagtactttgcttgcagtggaagagtgacatctttgttatctaaaaaaaaaaaaaaaagcggcggcggcggcggcggcgcagcatcgaagatgatcatccccgtgcgctgcttcacctgcggcaaggtgatcgggaacaagtgggaccactacctcgatcttctccaggccgattacacggagggggatgctctggacgccttgggcttggttcgctactgctgccggcgtatgctcatgacccatgttgaccttatcgagaagttgctcaactacaacacgctggagaaaacggagacaacttaattgaacccagcctgctgcgaaacccttgttcctcttacttctcagacatgggtgtgagactgtgaggtcctaatgcgaagttggttcgttcctgaatgtccaaagactgttagtgtgacaatgtgtttgtcctatgtagctttcggtactattaacgaacatatgtatgcactttccggtaccaaacctatgaagttctaagccttggaattacaaatctgtgttttctgtttaaaaaaaaaaaaaaaaaa

SEQ ID NO: 16 - DNA, 612 bp, Populus tremula
  misc_feature (466)..(466): n is a, c, g, or t
gtttctaccagcaggtccaattgcacttctgccaattggagacaaattcagcaatattgtttggaccatgaacccagaggagtcatctgattttaaatcaatgaaagaggatgattttgtgaaagctgtaaatcatgctctggattatggatatggttctcatcctaagtcaagtctaccgggcagtgcaggcatgttttcatggctcggaggaaatgtgacaatatttgctaatgagtcttttgaaattccaccaaaagtggtcaagttggcatctgagaggatggcatttccattgtctttaatgcatgcaaatgaatatgcatcaaagcgtgttgttctgattggtgatgcagcacacacagtacatcctttagctggccagggagttaatttgggttttggagatgcatttgctctttcaagaatcattgctgaaggaattgctgtgggcatggacattggnggaggtgatcgttgttgaaaaaatatgaagcagagagaaaactggcaaacatgactatgatggctattctagatggctttcagaaggcatactccgttgattttggccctctaaatatactacgtgctgctgcacttcatggggca

SEQ ID NO: 17 - DNA, 567 bp, Populus tremula x Populus tremuloides
  misc_feature (504)..(504): n is a, c, g, or t
ggaccatgaacccagaggagtcatctgactttaaatcaatgaaagaggatgattttgtgaaagctgtaaatcatgctctggattatggatatggttctcatcctaagtcaagtctaccgggcagtgcaggcatgttttcatggctcagaggaaatgtgacaatatttgctaatgagtcttttgaaattccaccaaaagtggtcaagttggcatctgagaggatggcatttccattgtctttaatgcatgcaaatgaatatgcatcaaagcgtgttgttctgattggtgatgcagcacacacagtacatcctttagctggccagggagttaatttgggttttggagatgcatttgctctttcaagaatcattgctgaaggaattgctgtgggcatggacattggggaggtatccttgttgaaaaaatatgaagcagagagaaaactggcaaacatgactatgatggctattctagatggctttcagaaggcatactccgttgatnttggccctctaaatatactacgtgctgctgcacttcatggggcacactttatttcgccactca

SEQ ID NO: 18 - DNA, 457 bp, Gossypium hirsutum
gctttcgagcggccgcccgggcaggtacagtgtgagctgcatcgccaattagagcaacgcgctttgacgcatagtcattggcatgccttaaggataatgggaaagccattctttcagaggccaatttgactacttttggtgggatttcaaagcaatcattagcagacttggagatacctcctttaagccaagaaaataagtctgaactacccaatatgcttggtgtaggatgaggaccataaccataatccagagcatgatttacagctttcagaaagttatcttcggttattgatttaaactctgatgactctttcgggctcatagtccaaacgatattgctaaacttatcacctattggcagaagagcaagcggaccagaaggtagaaacctctgccaggcacaatggttttctacatcatgttcaactgtacctcggccgcgaccacgctaagc

SEQ ID NO: 19 - DNA, 670 bp, Solanum tuberosum
cgtagcaacagcaagcatgaacaggctttctgttagcagaagaattgtgtcgaatattcatggattcagatctgtacagaggactttatgcagtgatacaggtgttaaaaatcccgatttggtcagtgaaaagcctttggaaaagcctggattaacagatgacataccgcaatatgatattgctattgttggaggtggaatggttgggatggctctagcgtgctccttagcaactatgccattgacaaagcaactaagtgttgccatcattgacagtaatcctgctttgatgaataactttcacatcaagaaagaggaccccccagatccacgggtcagtacagtcacacctgccaccatttctttcttcaaagatatgggtgcatgggaatttgttcagcagcaccgtcatgcttacttcgataaaatgcaggtttgggattatactggtctgggctatacaaggtacaatgcaagagatgttgatgcagatgttctcgggtaagaattccttttattgaggtaacaacttgctttcatagttatgatgttcttgattccagatgatgcttgagttttgtctttaggtgcgtggtggagaataaggtgctacacaaatccctgctatcatgcatacagaatacggattttcagaagacaatacactcgt

SEQ ID NO: 20 - DNA, 1991 bp, Arabidopsis thaliana
tgttaaagtttcaatcttttgtcggtgatctcatgtttgttgtgtctgtcaagttctgcaatctcttaactttataggaaattggatcccttgtttgttttgtgcaatttagattagagtccatcttggaatgttgaatttgtatctatttattactatccagcttacttggatattagagaagattattatataatgatgctatgtctaaggacagggtgctcgcaaggaatcttaggacaagtgtctgtggaacaaggattttgcagaggacttgttcgactgttgtgacacatgatgtttctacttcttgttccaatgagagcaatgcattaaataattccactgacgatggtccacaacatgacattgctattgttggtggtggaatggttggaatcgctttagctgcttcattggccagcaagccattgacaaagcacttgaatgtcgctattattgacaataatcctcttttggggagaagaaatatcatagagaaagggcatcaacctgatcccagagtcagtacagtcacacctgcaacaatatccttcctcaaagatattggtgcctggaaatacattgagcaacagcgacatgcgtattttgataagatgcaggtatgggactatacaggccttggatatacaagatacaatgctaatgatgtacatcaagatgttttggggtgtgtagtggagaataaagttcttcagagttctcagctatcttgtgtacaggaatcagatctccagaagactgtttatcccgcgaggttaaatgcgatggatatgctacctagctcttcgttgacaggtcttggtgaagtaccatccacaacagacttgttcatgcgaggacgccttgctaagctggagctaagcgatggaaatcgcgtctacgcaaaactggtggtaggcgctgatgggtctaagtcgcgagtaagggagttggctgggatcaaaacgactggatggaactactcacagaacgctataatttgtactgttgagcatacagccgaaaactacacagcatggcaacggtttttacccaatggcccaatcgcacttcttcccattggtgacaagtttagcaacatagtatggaccatggacccaaaagaggcatcagatcgaaaattgatgaacgaggatgacttcatcaaagctgtaaatgatgctctggattctggatatggtcctaatccagagactacaagttcacgggatagtctctcttggcttacaggagatgcgaccatatctgccaaagagaggtttgaaactcctcccaaggttgtgaagctttcttcagagagaatgatgttccctttgtctttaaggcacgcaaaagactatgtgtcaaaacgtgtggctcttgttggtgactcagctcatacagttcacccgttggctggtcaaggagtaaatctaggatttgcagatgcgtgtgctctctccaaagctatagcagagggcattgctcttggtactgatataggcgaggcgaatctgctcaaacgttatgaagcagagagaaaacctgcaaatatagcaatgatggcggttttagacggaatccagaagatgtactcgttggatttcggaccgctgaatgctctaagagcagctgcattccacggggctcactacatttcgccgcttaaaaagcgtatcatatcttatgcttcaggagagcaatctttgccgctattttcttgaagaatgcgagggattgttcgctttgttctcaaaatactctgcatcatctcttcttttgccttatatagaaatttaatggtgactgaaaacagctcccagagcccatgttttaaataagtctgttgtatttagttttttatgtttcagtcaataaaatactaaaatgttacataatctgatatcaaaacaatgtaaaatgtgaatactgtgatttaaattttgatgtccaacactgccacataaggaaggcaaagtcattgttcc

SEQ ID NO: 21 - DNA, 745 bp, Arabidopsis thaliana
cctggaaatacattgagcaacagcgacatgcgtattttgataagatgcaggtatgggactatacaggccttggatatacaagatacaatgctaatgatgtacatcaagatgttttggggtgtgtagtggagaataaagttcttcagagttctcagctatcttgtgtacaggaatcagatctccagaagactgtttatcccgcgaggttaaatgcgatggatatgctacctagctcttcgttgacaggtcttggtgaagtaccatccacaacagacttgttcatgcgaggacgccttgctaagctggagctaagcgatggaaatcgcgtctacgcaaaactggtggtaggcgctgatgggtctaagtcgcgagtaagggagttggctgggatcaaaacgactggatggaactactcacagaacgctataatttgtactgttgagcatacagccgaaaactacacagcatggcaacggtttttacccaatggcccaatcgcacttcttcccattggtgacaagtttagcaacatagtatggaccatggacccaaaagaggcatcagatcgaaaattgatgaacgaggatgacttcatcaaagctgtaaatgatgctctggattctggatatggtcctaatccagagactacaagttcacgggatagtctctcttggcttacaggagatgcgaccatatctgccaaagagaggtttgaaactcctcccaaggttgtgaagctttcttc

SEQ ID NO: 22 - DNA, 632 bp, Oryza sativa
gaaaagatgatctaccctactagactgatttctcttgctttgccatcaaagagcagacaagcaccaacaagggaagcaatatccagtggttaccctccagaagaattaaatcgcagcaatttagttaaacttgagctaagtgatggacaaactttatattcaaaactagtggtgggagctgacggttccaagtccaatgtaaggcaaacagcaggcataaaaacaacagggtggaattatcctcaaagtgcaattatttgtacagtggagcatattaccgaaaacgattgtgcatggcagaggtttctcccttctgggccaattgctctacttccaataggtgacaactacagcaatattgtgtggacaatgagcccagaggagtcattgcgccataagtcaatgagtcctgaagattttgtgaagtctgtgaacaatgcattggattttggttacggtccacatccacactctggtagtcttgactattacatggaaaagttattttctgacattggaagtactgctgcatctacaaaggaatgctttgaagtacctccaaaagcaatcggggtagtctgtgagagaatggcatttccattgtcgctgaagcattcacatgattatatttc

SEQ ID NO: 23 - DNA, 544 bp, Oryza sativa
tctgtgagagaatggcatttccattgtcgctgaagcattcacatgattatatttcaaaaaggttagcactagtcggtgatgcagctcatacagtccatcccttagctggccaaggtgtcaacttaggttttggagatgcagctgctttagcaaaagttatcgctgaaggagtttctgtgggtgctgattttggggatatttccttgctcaaaaggtatgagaataatcgcaaagcagccaatgttgcaatggcagcgctgctagatggtttccagaagatgtactctgttgattttggacccctcaatgtgcttagggctgcagcgtttcacggtgcccagtacatatcaccactgaagaagaacataatctcgtatgcaatgggcgacgcgaaatggccgctgttttcatagacagtagatccggaaactttaaatttgttagttgttactgcagtgtcgatgtgttattctaaattagtatagtgtactttgtctccagtgttctcatccagagtagtaatgcatcacaggcacacgttgca

SEQ ID NO: 24 - DNA, 807 bp, Oryza sativa
  misc_feature (732)..(732): n is a, c, g, or t
ggacgagacaagggggattttggttacggtccacatccacactctggtagtcttgactcttacatggaaaagttattttctgacattggaagtactgctgcatctacaaaggaatgctttgaagtacctccaaaagcaatcggggtagtctgtgagagaatggcatttccattgtcgctgaagcgttcacatgattatatttcaaaaaggttagcactagtcggtgatgcagctcatacagtccatcccttagctggccaaggtgtcaacttaggttttggagatgcagctgctttagcaaaagttatcgctgaaggagtttctgtgggtgctgattttggggatatttccttgctcaaaaggtatgagaatgatcgcaaagcagccaatgttgcaatggcagcgctgctagatggtttccagaagatgtactctgttgattttggacccctcaatgtgcttagggctgcagcgtttcacggtgcccagtacatatcaccactgaagaagaacataatctcgtatgcaatgggcgacgcgaaatggcccgctgttttcatagacagtagatccggaaactttaaatttgttagttgttactgcagtggtcgatgtgttattctaaattagtatagtgtactttgtctctagtgttctcatccagagtagtaatgcatcacaggcacacgttgcagagaagaaatgtagctagaaagaaataaattggatctnctnggtgatctangattacccttggaatgtacctcctcttgcactncagtaactcgctttntgntttgacccaca

SEQ ID NO: 25 - DNA, 1732 bp, Oryza sativa
aaactcgcaaaccaaacccggcgacggcacacaggttttagcaccggacgggagctctccgccgtcgccgacgacgacgccgccggggtgcccagctcgcaggatcacacagagaaggttgatgatgtgaaagctagacccgacgagctcgacattgctatcgttggtggaggcatggtgggattggctgttgcttgtgcattgtccaatatgccattgactaaacatctgagggttggcatcattgatagcaatcctgcattgaagtcaagaaattatctgaagaaagatggtgtacctgattcaagggtcagtactattactcccgcgaccatatccttcttcagagatattggtgcatgggatcatgttcaacagcaaagacacgctttctttggtaaaatgcaggtatgggattacactggactgggttacacaaggtatagtgcaagggatgtgggaaaagaatatctcggatgcgtggtggagaacaaggtgctttgcaactcactgttgttacgattacaggaggaaaatggagacattgaaaagatgatctaccctactagactgatttctcttgctttgccatcaaagagcagacaagcaccaacaagggaagcaatatccagtggttaccctccagaagaattaaatcgcagcaatttagttaaacttgagctaagtgatggacaaactttatattcaaaactagtggtgggagctgacggttccaagtccaatgtaaggcaaacagcaggcataaaaacaacagggtggaattatcctcaaagtgcaattatttgtacagtggagcatattaccgaaaacgattgtgcatggcagaggtttctcccttctgggccaattgctctacttccaataggtgacaactacagcaatattgtgtggacaatgagcccagaggagtcattgcgccataagtcaatgagtcctgaagattttgtgaagtctgtgaacaatgcattggattttggttacggtccacatccacactctggtagtcttgactattacatggaaaagttattttctgacattggaagtactgctgcatctacaaaggaatgctttgaagtacctccaaaagcaatcggggtagtctgtgagagaatggcatttccattgtcgctgaagcattcacatgattatatttcaaaaaggttagcactagtcggtgatgcagctcatacagtccatcccttagctggccaaggtgtcaacttaggttttggagatgcagctgctttagcaaaagttatcgctgaaggagtttctgtgggtgctgattttggggatatttccttgctcaaaaggtatgagaataatcgcaaagcagccaatgttgcaatggcagcgctgctagatggtttccagaagatgtactctgttgattttggacccctcaatgtgcttagggctgcagcgtttcacggtgcccagtacatatcaccactgaagaagaacataatctcgtatgcaatgggcgacgcgaaatggccgctgttttcatagacagtagatccggaaactttaaatttgttagttgttactgcagtgtcgatgtgttattctaaattagtatagtgtactttgtctccagtgttctcatccagagtagtaatgcatcacaggcacacgttgcagagaagaaatgtagctagaaagaaataaattggatctcctggtgatctaggattaccttgaatgt

SEQ ID NO: 26 - DNA, 1732 bp, Oryza sativa
aaactcgcaaaccaaacccggcgacggcacacaggttttagcaccggacgggagctctccgccgtcgccgacgacgccgccgccggggtgcccagctcgcaggatcacacagagaaggttgatgatgtgaaagctagacccgacgagctcgacattgctatcgttggtggaggcatggtgggattggctgttgcttgtgcattgtccaatatgccattgactaaacatctgagggttggcatcattgatagcaatcctgcattgaagtcaagaaattatctgaagaaagatggtgtacctgattcaagggtcagtactattactcccgcgaccatatccttcttcagagatattggtgcatgggatcatgttcaacagcaaagacacgctttctttggtaaaatgcaggtatgggattacactggactgggttacacaaggtatagtgcaagggatgtgggaaaagaatatctcggatgcgtggtggagaacaaggtgctttgcaactcactgttgttacgattacaggaggaaaatggagacattgaaaagatgatctaccctactagactgatttctcttgctttgccatcaaagagcagacaagcaccaacaagggaagcaatatccagtggttaccctccagaagaattaaatcgcagcaatttagttaaacttgagctaagtgatggacaaactttatattcaaaactagtggtgggagctgacggttccaagtccaatgtaaggcaaacagcaggcataaaaacaacagggtggaattatcctcaaagtgcaattatttgtacagtggagcatattaccgaaaacgattgtgcatggcagaggtttctcccttctgggccaattgctctacttccaataggtgacaactacagcaatattgtgtggacaatgagcccagaggagtcattgcgccataagtcaatgagtcctgaagattttgtgaagtctgtgaacaatgcattggattttggttacggtccacatccacactctggtagtcttgactattacatggaaaagttattttctgacattggaagtactgctgcatctacaaaggaatgctttgaagtacctccaaaagcaatcggggtagtctgtgagagaatggcatttccattgtcgctgaagcattcacatgattatatttcaaaaaggttagcactagtcggtgatgcagctcatacagtccatcccttagctggccaaggtgtcaacttaggttttggagatgcagctgctttagcaaaagttatcgctgaaggagtttctgtgggtgctgattttggggatatttccttgctcaaaaggtatgagaataatcgcaaagcagccaatgttgcaatggcagcgctgctagatggtttccagaagatgtactctgttgattttggacccctcaatgtgcttagggctgcagcgtttcacggtgcccagtacatatcaccactgaagaagaacataatctcgtatgcaatgggcgacgcgaaatggccgctgttttcatagacagtagatccggaaactttaaatttgttagttgttactgcagtgtcgatgtgttattctaaattagtatagtgtactttgtctccagtgttctcatccagagtagtaatgcatcacaggcacacgttgcagagaagaaatgtagctagaaagaaataaattggatctcctggtgatctaggattaccttgaatgt

SEQ ID NO: 27 - DNA, 787 bp, Zea mays
cactgaaatagtgaccatttcgtgtcacccgttgcataaaagatgatgttcttttttagtggtgatatgtactgggcaccgtggaacgcagcagctctcactacattaaggggccccaaatcaacagagtacatcttctgaaaaccatctagcactgctgccatcgccacattggcagccttcctctccttctcataccgctgtagcaaagtcagatccccaacatccgagcccactgaaactccctcagcaataacttttgctaaagcagcagcatcaccaaagcccaagtttacaccttggccggctaaggggtggacagtatgagcagcatctccaactaatgctaaccttttggaaacataatcatgggaatgcatcaacgacaatggaaacgccattctctcaaaaactacacgggttgctttcggtggcacttcgaagcattcctttgtagatgctgcactgctcccaatgccagaaaagaactgttccatgtaatggtcaagagcggtagagtgtggatgcggaccataaccaaaatccaatgcgtggttcacagaattcacaaactcttctgggctcattgacttatggcgcagtgactcctctggactcattgtccatacaatgttgctgaagttattgcctatcggaagtagcgcaattggaccagaagggagaaacctctgccacgcacaatcattttccacaacatgctctactgtacagataattgcactctgcggataattccaactagttgtttttatgcctgcaatctgtc

SEQ ID NO: 28 - DNA, 802 bp, Zea mays
caaagcagtgctgttcactgaaatagtgaccatttcgtgtcacccgttgcataaaaaatgatgttcttttttagtggtgatatgtactgggcaccgtggaacgcagcagctctcactacattaaggggccccaaatcaacagagtacatcttctgaaaaccatctagcactgctgccatcgccacattggcagccttcctctccttctcataccgctgtagcaaagtcagatccccaacatccgagcccactgaaactccctcagcaataacttttgctaaagcagcagcatcaccaaagcccaagtttacaccttggccggctaaggggtggacagtatgagcagcatctccaactaatgctaaccttttggaaacataatcatgggaatgcatcaacgacaatggaaacgccattctctcaaaaactacacgggttgctttcggtggcacttcgaagcattcctttgtagatgctgcactgctcccaatgccagaaaagaactgttccatgtaatggtcaagagcggtagagtgtggatgcggaccataaccaaaatccaatgcgtggttcacagaattcacaaactcttctgggctcattgacttatggcgcagtgactcctctggactcattgtccatacaatgttgctgaagttattgcctatcggaagtagcgcaattggaccagaagggagaaacctctgccacgcacaatcattttccacaacatgctctactgtacagataattgcactctgcggataattccaactagttgtttttatgcctgcaatctgtc

SEQ ID NO: 29 - DNA, 792 bp, Zea mays
cgaacgaaattactgattgcgttctttgtttccagtacatgccgaaacaaagaacgaggtgaggacaagagtttcaaagcagtgctgttcactgaaatagtgaccatttcgtgtcacccgttgcataagagatgatgttcttttttagtggtgatatgtactgggcaccgtggaacgcagcagctctcactacattaaggggccccaaatcaacagagtacatcttctgaaaaccatctagcactgctgccatcgccacattggcagccttcctctccttctcataccgctgtagcaaagtcagatccccaacatccgagcccactgaaactccctcagcaataacttttgctaaagcagcagcatcaccaaagcccaagtttacaccttggccggctaaggggtggacagtatgagcagcatctccaactaatgctaaccttttggaaacataatcatgggaatgcatcaacgacaatggaaacgccattctctcaaaaactacacgggttgctttcggtggcacttcgaagcattcctttgtagatgctgcactgctcccaatgccaaaaaagaactgttccatgtaatggtcaagagcggtagagtgtggatgcggaccataaccaaaatccaatgcgtggttcacagaattcacaaactcttctgggctcattgacttatggcgcagtgactcctctggactcattgtccatacaatgttgctgaagttattgcctatcggaagtagcgcaattggaccagaagggagaaacctctgccacgcacaat

SEQ ID NO: 30 - DNA, 565 bp, Sorghum sp.
tactgctcttgaccgttacatggaacagttcttttctggcattgggaacacggcagcatctacaaaggaatgctttgaagtaccaccgaaagcaaccggtgtagtttctgagagaatggcatttccattgtcattgatgcattcccatgattatgtttccaaaaggctagcattagttggagacgctgctcatactgtccaccccttagccggccaaggcgtcaacttgggctttggtgatgctgctgctttagcaaaagttattgctgaaggagtttctgtgggctcagatgttggggacctgactttgctacaacggtatgagaaggaccggaaggctgccaatgtggcgatgacagcagtgctagatggtttccagaagatgtactctgttgattttgggccccttaatgtagtgagagctgctgcattccacggtgcccagtacatatcaccactgaaaaagaacatcatatcttatgcaatgggtgacaccaaatggccactatttcggtgaacagtactgctttaaactgttgttctgaacttgtgcgtttcggattgt

SEQ ID NO: 31 - DNA, 989 bp, Zea mays
  misc_feature (471)..(471): n is a, c, g, or t
ctgctttgctcccgttcccatgctgatttttttaggcggctgctggcctgcctgtctccactggaagctccggattacgtaggaaaggggcgggcggcatgaggatccagtgcgacgcgtgcgaggccgcggcggccacggtggtgtgctgcgccgacgaggcggcgctgtgcgcgcgctgcgacgtggagatccacgccgccaacaagctcgccagcaagcaccagcggctcccgctcggcgacgccgcggcgctccccgccagcctcccgcgctgcgacgtgtgccaggagaggccggccttcatcttctgcgtcgaggacagggcgctcctctgccgcgactgcgacgagcccatccacgtccccggcacgctctccggcaaccaccagcgctacctcgccaccggcatccgcgtcggcttcagctccgtctgcggcgccagcgccgagggcctcccgccggcgccgnccaagggcagctccaagcccgcggccgtcatcagcgcgccagctgccgggacgaccaccaaaacaaggacggtcaaggacgcgctgccacaggaggtgccgtcctcgccgttcctccccccatccggttgggccgtcgaggacctcctcgagctgtcggactacgaatccagcgacaagaaagactcgtctcttgggctcaaggagctggagtggttcgaggacatcgacctgttccacgcccattcgccagctaaaactgccatggcggagttcttcgcctcgccgcagccagtgagtaacgccgggttctacaaggcgaacggggcgcgcctgttcaagaagtcacggatggaggtgcctgacgacgacgacgaggactacttcatcgttcctgatcttggctgagatgagagatctctgcatgattctccagtcatgtgatgtgtgtatgtatgcaaaatgggtatgtataccatcatggggtgcactgacgcttccatagggcatatgtaatca

SEQ ID NO: 32 - DNA, 516 bp, Zea mays
gggagagaaaaaggggagcccgatccaatcccaatcccctcccgccgcgtcacatcgccgccgtcctcgtgccccgttacctctaaaaccctagcggggagccgccgagccggggatgaagatccagtgcaacgcgtgcggggccgcggaggcgcgggtgctgtgctgcgcggacgaggccgcgctctgcgtcgcctgcgacgaggaggtgcacgccgccaacaagctcgccgggaagcaccagcgggtgccgctcctgacggacgccgccgccgctgccgccgcagcggcgccggccgtgcccaagtgcgacatctgtcaggaggcttctggatacttcttctgcctagaggaccgtgcactactttgtagagactgtgatgttgctatacacacagtgaattcctttgtttcagtacatcagagattcctgctaactggagttcaggtgggtcttgatcctgctgatccagttccacctattgctgacaagcatgttaatgctgttggtggatc

SEQ ID NO: 33 - DNA, 862 bp, Zea mays
tgacactatagaacaagtttgtacaaaaaagcaggctggtaccggtccggaattcccgggacgatccaatcccaatcccctcccgccgcgtcacatcgccgccgtcctcgtgccccgttacctctaaaaccctagcggggagccgccgagccggggatgaagatccagtgcaacgcgtgcggggccgcggaggcgcgggtgctgtgctgcgcggacgaggccgcgctctgcgtcgcctgcgacgaggaggtgcacgccgccaacaagctcgccgggaagcaccagcgggtgccgctcctgacggacgccgccgccgctgccgccgcagcggcgccggccgtgcccaagtgcgacatctgtcaggaggcttctggatacttcttctgcctagaggaccgtgcactactttgtagagactgtgatgttgctatacacacagtgaattcctttgtttcagtacatcagagattcctgctaactggagttcaggtgggtcttgatcctgctgatccagttccacctattgctgaaaagcatgttaatgctgttggtggatcagtgaatcaaccagtgagacatcagccaaggagaagcccgacagttcaattttcagttgaagggagtgcttctgttcctaccaaaaatgtaacaaatggagattgttcaaggcagaattttgtcccaacagccagggcagaagtggttgattggactatgaataatagtacaattcgatcagtagaatctccgcctaagtacatttcagaggagagccccacacttctgcaatctagccagaccacaacagtcttctccaaccaaattaatggcaatagtgatggggcctaccacttgtcattct

SEQ ID NO: 34 - DNA, 832 bp, Sorghum bicolor
cgcattggctcctcgcaaccatatctccctcctctcgctttattacaaagctttactattagctcacgcgcgccgtgcgggagagagagagagagaggagccgatccaacccctcccgtcgcgtcccctcgccgccgtcctcgtccctctcccctcccctccctctaaaaccctagctagcgaggagccaggggatgaagatccagtgcaacgcgtgcggggccgcggaggcgcgggtgctgtgctgcgcggacgaggccgcgctctgcgtcgcctgcgacgaggaggtgcacgccgccaacaaactcgccgggaagcaccagcgggtgccgctcctgacggacgccgacgccgctgggaccgccgccgcggcgccggccgtgcccaagtgcgacatctgtcaggaggcttctggatacttcttctgcctagaggaccgtgcactactttgtagagactgtgatgttgctatacacacagtaaattcctttgtttcagtacaccagagattcctgctaactggagttcaggtgggccttgatcctgctgatccagttccacctattgctgaaaagcatgttaatgccgctggtggatcagtaaatcaaccagtgaaacatctgccaaggagaagcccgacagttcaattttcagttgaaggcagtgcttctgttccaagcaaaaatgtaacacatggagattattcaaggcagaattctgttccaacagccagggcagaagtggttgattggactatgaacaatagtacaattcgttcagtagaatccccgcctaagtacatgtcagaggaaagtccaacacttctgcaatc

SEQ ID NO: 35 - DNA, 787 bp, Sorghum bicolor
ccatatctccctcctctcgctttattacaaagctttactattagctcacgcgcgccgtgcgggagagagagagagagaggagccgatccaacccctcccgtcgcgtcccctcgccgccgtcctcgtccctctcccctcccctccctctaaaaccctagctagcgaggagccaggggatgaagatccagtgcaacgcgtgcggggccgcggaggcgcgggtgctgtgctgcgcggacgaggccgcgctctgcgtcgcctgcgacgaggaggtgcacgccgccaacaaactcgccgggaagcaccagcgggtgccgctcctgacggacgccgacgccgctgggaccgccgccgcggcgccggccgtgcccaagtgcgacatctgtcaggaggcatctggatacttcttctgcctagaggaccgtgcactactttgtagagactgtgatgttgctatacacacagtaaattcctttgtttcagtacaccagagattcctgctaactggagttcaggtgggccttgatcctgctgatccagttccacctattgctgaaaagcatgttaatgccgctggtggatcagtaaatcaaccagtgaaacatctgccaaggagaagcccgacagttcaattttcagttgaaggcagtgcttctgttccaagcaaaaatgtaacaaatggagattattcaaggcagaattctgttccaacagccagggcagaagtggttgattggactatgaacaatagtacaattcgttcagtagaatccccgcctaagtacatgtc

SEQ ID NO: 36 - DNA, 645 bp, Saccharum officinarum
gagagaaagggggagccgggagccgatccagtccctcccgccgcgtcccctcgcctcgccgccgtcctcgtccccttcccctccctctaaaaccctagcggggagccagccggggatgaagatccagtgcaacgcgtgcggggccgcggaggcgcgggtgctgtgctgcgcggacgaggccgcgctctgcgtcgcctgcgacgaggaggtgcacgccgccaacaagctcgccgggaagcaccagcgggtgccgctcctgaccgacgccgccgggaccgccgcgccggccgtgcccaagtgcgacatctgccaggaggcttctggatacttcttctgcctagaggaccgtgcactactttgtagagactgtgatgttgctatacacacagtgaattcctttgtttcagtacatcagagattcctgctaactggagttcaggtgggtcttgatcctgctgatccagttccacctattgctgaaaagcatgttaatgccgctggtggatcagtaaatcaaccagcgaaaaatctgccaaggagaagcccgacagttcaaggcagtgcttctgttccaagcaaaaatgtaacaaatggagattattcaaggcagaattctgttccaacagcccaggcagaagtgggtga

SEQ ID NO: 37 - DNA, 595 bp, Saccharum officinarum
gagccgatccagtccctcccgccgcgtcccctcgcctcgccgccgtcctcgtccccttcccctccctctaaaaccctagcggggagccagccggggatgaagatccagtgcaacgcgtgcggggccgcggaggcgcgggtgctgtgctgcgcggacgaggccgcgctctgcgtcgcctgcgatgaggaggtgcacgccgccaacaagctcgccaggaagcaccagcgggtgccgctcctgaccgacgccgccgggaccgccgcgccggccgtgcccaagtgcgacatctgccaggaggcttctggatacttcttctgcctagaggaccgtgcactactttgtagagactgtgatgttgctatacacacagtgaattcctttgtttcagtacatcagagattcctgctaactggagttcaggtgggtcttgatcctgctgatccagttccacctattgctgaaaagcatgttaatgccgctggtggatcagtaaatcaaccagcgaaaaatctgccaaggagaagcccgacagttcaaggcagtgcttctgttccaagcaaaaatgtaacaaatggagattattcaaggcagaatt

SEQ ID NO: 38 - DNA, 1169 bp, Triticum aestivum
gcacgagcttatctcttctccggctcagcctagcaaaagcgctctccggccagcgagaaagcaagaaggcggaggcatgaagatccagtgcgactcgtgcggcgtcgcggcggccacggtggtgtgctgcgcggacgaggcggcgctgtgcgcgcgctgcgacgtggagatccacgccgctaacaagctcgccagcaagcaccagaggctcccgctcgacgcgctcggcgccaagctcccgcgctgcgacatctgccaggagaaggccgccttcatcttctgcgtcgaggaccgggcgctcttctgccgcgactgcgacgagcccatccacgtccccggcacgctctccgggaaccaccagcgctacctcgccaccggcatccgcgtcggcctcggccccgtctccgcctgcgccgccggggaccacggcgccgcccacgacgccgaccaccacgccccgcccaaggtctcctgcgagccccagcccgccgcccgcgcgcagcaggtcccctcgccgccgcagttcctgccgcaggactgggccgtcgacgagctcctgcagttctcggactacgagtccagcgacaagctgcacaaggactcgcctctcgccttcaaggagctggagtggttcaccgcggacatggagctcttccacgaccacgcgcccaagggcggcagggcgaccatggaggtccccgagttcttcgcctcgcaggcggcagacgacgcgggctactacaggccgagcagagtcgccgccaccgccggcgtgcgccagagcaagaaggcccgcgtcgagatccccgacgacgaggacttcttcatcgtgcctgatcttggctaagcgatgctcctagatgctagtacctcctcgtgtgattttgcagtgttgttgtatacagagaggagctgtgtacaaaagctagtcttctgtcgaactgtgcccctgtgcatcttgacgtagcatcgctgaactgtgacatataataattattatcgtaagatatgagctttgcaaaatggacgcttttgatgtacctctgtacttttgcactagttagagcatctctagtggcttgtgtaaatttggtagtctatatcttcacatacacaatggtgtaattctttttcattttagacaacctcagtttttcagtggattgtct

SEQ ID NO: 39 - DNA, 840 bp, Triticum aestivum
  misc_feature (599)..(599): n is a, c, g, or t
caccagctattgagatataggcctatccaggtgagatgatataacaagtttgtacaaaaaagcaggctggtaccggtccggaattcccgggatatcgtcgacccacgcgtccgccgtatcctcccttcccctcccctcccctcccccccctttcccgaggcagcgggcgagagagaacagagcggagcagcaaagagaggccgtatctggcgcgggcgatgaagatccagtgccgcgccgcccaagtgcgacatctgccaggaggcttctggatacttcttctgcctggaagaccgtgctctactttgtagagattgtgatgttgctatacacacagtgaattcctttgtttcagcacatcaaaggttcctgctgacaggagttcaagttggcctcgatcctgctgatcctgttccacctgttgctgacaagcacgtaaacacttcaggtggatcagtggattcactaccgaaacacttgccaaagaaaaatcctacagtcctattttcaggtgaaaactgtgcatctattcccagccagaatgcaatcagtggagattattcgaggcagttctgtcaatacacacagaggacactgtngttatgacacatggcttagatggggagcgcccctaccactgcagaannnnggatgtaatcaaattttgtctctatcagacacacagcttatccaccaatgacagaatgtgccgggcttacattacattctaggcggaatggtcagacgtttactgatgggcggggatgaatctcaagaactagatatgtggcaattgggttactggcatggttctcagggttgacatgctactgggaagtg

SEQ ID NO: 40 - DNA, 591 bp, Hordeum vulgare
cctcgaggccccccgcccccctcccctcccctcccttcccctttcccgaggcagcgagcgagcgagagagcagagcagcgcggggcaggcggggaaaacgatgaagatccagtgcaacgcgtgcggggcggcggaggcgcgggtgctctgctgcgccgacgaggccgcgctctgcgccgcctgcgacgaggaggtgcacgccgccaacaggctcgccgccaagcaccagcgcgtgccgctcctccccgacgccgcgcccaacgccgccgcgccgcccaagtgcgacatctgccaggaggcttctggatacttcttctgcctggaggaccgtgctctactttgtagagattgtgatgttgctatacacacagtgaattcctttgtttcagcacatcaaaggttcctgctgacaggagttcaagttggcctcgatcctgccgatcctgttccacctgttactgaaaagcatgttaacacttctagtggatcagtggattcacaaccaaaacacctgccaaagaaaaatcctacagtcctggtttcaggtgaaatcggtggatctattcccagccaaaatgcaatcagtgaggg

SEQ ID NO: 41 - DNA, 643 bp, Panicum virgatum
ccacgcgtccggatccagtcccatccggccgaatcccctcgccgccgccgccctctccttcctctaactaaaaccctagcgtgcgtgcgcggggatgaagatccagtgcaacgcgtgcggggccgcggaggcgcgggtgctgtgctgcgcggacgaggcggcgctctgcggggcctgcgacgaggaggtgcacgccgccaacaagctcgcggggaagcaccagcgggtgccgctgctctcggacgccgacgccgccgccgcggcggcggcgcccgccgtgcccaagtgcgacatctgccaggaggcttctggatacttcttctgcctagaggaccgtgcactactttgtagagattgtgatgttgctatacacacggtaaattcatttgtttcggtgcatcagagattcctgctaactggagttcaggtgggccttgatcctgccgatccagttccgcccattgctgaaaaccgtgttaatgacgctggtggctcggtatatcaaccagcaaaacatctgccaaggagaaacccaacagttcaattttcaggtgaaggcactgcttctgttcccagcaaaaatttaataaatggagactattcaagacagaattctgttccaacagtcaggacgggagtggtt

SEQ ID NO: 42 - DNA, 590 bp, Secale cereale
cagtgcgccgcccaagtgcgacatctgccaggaggcttctggatacttcttctgcctggaggaccgtgctctactttgtagagattgtgatgttgctatacacacagtgaattcctttgtttcagcacatcaaaggttcctgctgacaggagttcaagttggcctcgatcctgccgatcctgttccacctgttgctgacaagcacgttaatacttcaggtggatcagtggattcactaccgaaacacttggcaaagaaaaaccctacaggcctattttcaggtgaaaacagtgcatctattcccagccaaagtgcaatcagtggagagtattcgaggcagatttctgctccaaataccaagacaggaatgaccaattggactatggacaacagtgcacttagattggcagagcctccacctaagtacctgtcagatggaaattcaaaacttttgctctctaatcagaccaccgcagctttatccagccaaatgagcagagatagtggccgggcttacaacttaccattctcaggcggtaatgggtcagacagtttacctgattggcctgtcgatgaatttttcagtaact

SEQ ID NO: 43 - DNA, 900 bp, Arabidopsis thaliana
atgaagattcagtgtaacgtttgtgaggcggcggaagcgacggttctatgttgcgccgacgaggctgctctttgttgggcttgcgatgagaaaattcacgccgctaataaactcgccggaaaacatcagagagtccctctctctgcctctgcctcttccatacccaaatgtgacatttgtcaggaagcatctggattcttcttttgtctgcaagatagagctttgctatgtaggaaatgtgatgttgcaatccacactgtgaatcctcatgtttcagctcaccagagatttcttctcactggaatcaaagttggtcttgaatctatagacactggtccttctactaaatcctcacctaccaatgatgataaaaccatggagaccaaaccttttgttcaatctatacctgagcctcaaaagatggccttcgatcatcatcatcaccagcagcagcaggaacagcaggaaggagttataccgggaactaaagtcaatgatcagacatcgacaaagcttcctctcgtaagtagcggatcaactactggaagcattcctcagtggcaaatagaggagattttcgggctaaccgactttgatcagagctatgaatacatggagaataatggatcatctaaggcggatactagtagacgaggagattcagacagttcttcgatgatgagatctgcagaagaagatggagaagataacaataactgcttgggaggtgagacatcatgggcggttccacagattcagtctccacctacagcgtctggtctaaactggcctaagcattttcaccaccactctgtgtttgttccggacataacttcttcaactccttataccggttcatccccgaatcaaagggttgggaaacggcggcgacggttctag

SEQ ID NO: 44 - DNA, 838 bp, Oryza sativa
cagtggagagataggggaagagagggagcgatccaatcgcgtcgctaaaaccctagcggctggctgatttgggggattgctcgtcggagagatgaagatccagtgcaacgcgtgcggcgcggcggaggcgcgggtgctgtgctgcgccgacgaggcagcgctctgcacggcgtgcgacgaggaggtgcacgccgccaacaagctcgccgggaagcaccagcgggtgccgctgctctccgacgacggcggcgccgcgcccgccgccgccgccccggccgtgcccaagtgcgacatctgccaggaggcttctggatacttcttctgcctggaggaccgtgcacttctttgcagagattgtgatgtttctatacacacagtaaactcctttgtttcagtacaccaaagattcctactaacaggtgttcaagttggccttgatcctgctgatccagttccacctgttgctgacaagcatgttaagagtgctggtggttcagtggattcagcaactaaacatttgcaaaggaatcctacagacttatctggtgaaaacagtgcatctttgcccagccaaaatgtaatcaatggtaattattctaggcagagttctgttacaatggccaagacaggacaggtcaattggactatgagcaacaacacaattagatcaatagaccctccacccaagtattcatcagaggaaagtccagcacttctgctagctagccacactagcaccatggcagcgtactccagtcaaatcagtaaggatagtgatcggatctacagcttaccattcacaggtggtaatgggtcagatagtctacatgattggcat

SEQ ID NO: 45 - DNA, 780 bp, Oryza sativa
gggaagagagggagcgatccaatcgcgtcgctaaaaccctagcggctggctgatttgggggattgctcgtcggagagatgaagatccagtgcaacgcgtgcggcgcggcggaggcgcgggtgctgtgctgcgccgacgaggcagcgctctgcacggcgtgcgacgaggaggtgcacgccgccaacaagctcgccgggaagcaccagcgggtgccgctgctctccgacgacggcggcgccgcgcccgccgccgccgccccggccgtgcccaagtgcgacatctgccaggaggcttctggatacttcttctgcctggaggaccgtgcacttctttgcagagattgtgatgtttctatacacacagtaaactcctttgtttcagtacaccaaagattcctactaacaggtgttcaagttggccttgatcctgctgatccagttccacctgttgctgacaagcatgttaagagtgctggtggttcagtggattcagcaactaaacatttgcaaaggaatcctacagacttatctggtgaaaacagtgcatctttgcccagccaaaatgtaatcaatggtaattattctaggcagagttctgttacaatggccaagacaggacaggtcaattggactatgagcaacaacacaattagatcaatagaccctccacccaagtattcatcagaggaaagtccagcacttctgctagctagccacactagcaccatggcagcgtactccagtcaaatcagtaaggatagtgatcggatctacaacttac

SEQ ID NO: 46 - DNA, 1450 bp, Oryza sativa
gatacagtggagagataggggaagagagggagcgatccaatcgcgtcgctaaaaccctagcggctggctgatttgggggattgctcgtcggagagatgaagatccagtgcaacgcgtgcggcgcggcggaggcgcgggtgctgtgctgcgccgacgaggcagcgctctgcacggcgtgcgacgaggaggtgcacgccgccaacaagctcgccgggaagcaccagcgggtgccgctgctctccgacgacggcggcgccgcgcccgccgccgccgccccggccgtgcccaagtgcgacatctgccaggaggcttctggatacttcttctgcctggaggaccgtgcacttctttgcagagattgtgatgtttctatacacacagtaaactcctttgtttcagtacaccaaagattcctactaacaggtgttcaagttggccttgatcctgctgatccagttccacctgttgctgacaagcatgttaagagtgctggtggttcagtggattcagcaactaaacatttgcaaaggaatcctacagacttatctggtgaaaacagtgcatctttgcccagccaaaatgtaatcaatggtaattattctaggcagagttctgttacaatggccaagacaggacaggtcaattggactatgagcaacaacacaattagatcaatagaccctccacccaagtattcatcagaggaaagtccagcacttctgctagctagccacactagcaccatggcagcgtactccagtcaaatcagtaaggatagtgatcggatctacaacttaccattcacaggtggtaatgggtcagatagtctacatgattggcatgttgatgagttctttagtaactcagaatttggctttgctgagcatggttcttctaagggtgacaacgctaagccagggagtgctggtggatctccgcagtgccgtctggctgaaggcctgtttgtcgaaggacttctaggtcaagtgcctgacaatccatggacagtgcctgaggtcccctcgccaccgacagcctctggtctctattggcaaaataatttgctttgcccttcgtacgacagcaccatgttcgtccctgagatttcctccttggagaactctcagaacaacttcactgtatctgctggtttgaagcgccgaaggaggcagttttgatctgccgatagcatcagacaccattcggtttagttggcaggtcacgttgagaggagggcttcttgcctgcctgaccaaagccacaaaacacagggatggatgatcatccagtggtcagtctaaaaatatgttgtaatgtttgtgtttcgactatacgtttgttgctgaagtttaaaggcgagggaattgttgtgagatctgggatgacagatttgttgttaccaattttgtgaatagtgaaaataaaaagagcatggataagatcatgaatt

SEQ ID NO: 47 - DNA, 1449 bp, Oryza sativa
atacagtggagagataggggaagagagggagcgatccaatcgcgtcgctaaaaccctagcggctggctgatttgggggattgctcgtcggagagatgaagatccagtgcaacgcgtgcggcgcggcggaggcgcgggtgctgtgctgcgccgacgaggcagcgctctgcacggcgtgcgacgaggaggtgcacgccgccaacaagctcgccgggaagcaccagcgggtgccgctgctctccgacgacggcggcgccgcgcccgccgccgccgccccggccgtgcccaagtgcgacatctgccaggaggcttctggatacttcttctgcctggaggaccgtgcacttctttgcagagattgtgatgtttctatacacacagtaaactcctttgtttcagtacaccaaagattcctactaacaggtgttcaagttggccttgatcctgctgatccagttccacctgttgctgacaagcatgttaagagtgctggtggttcagtggattcagcaactaaacatttgcaaaggaatcctacagacttatctggtgaaaacagtgcatctttgcccagccaaaatgtaatcaatggtaattattctaggcagagttctgttacaatggccaagacaggacaggtcaattggactatgagcaacaacacaattagatcaatagaccctccacccaagtattcatcagaggaaagtccagcacttctgctagctagccacactagcaccatggcagcgtactccagtcaaatcagtaaggatagtgatcggatctacaacttaccattcacaggtggtaatgggtcagatagtctacatgattggcatgttgatgagttctttagtaactcagaatttggctttgctgagcatggttcttctaagggtgacaacgctaagccagggagtgctggtggatctccgcagtgccgtctggctgaaggcctgtttgtcgaaggacttctaggtcaagtgcctgacaatccatggacagtgcctgaggtcccctcgccaccgacagcctctggtctctattggcaaaataatttgctttgcccttcgtacgacagcaccatgttcgtccctgagatttcctccttggagaactctcagaacaacttcactgtatctgctggtttgaagcgccgaaggaggcagttttgatctgccgatagcatcagacaccattcggtttagttggcaggtcacgttgagaggagggcttcttgcctgcctgaccaaagccacaaaacacagggatggatgatcatccagtggtcagtctaaaaatatgttgtaatgtttgtgtttcgactatacgtttgttgctgaagtttaaaggcgagggaattgttgtgagatctgggatgacagatttgttgttaccaattttgtgaatagtgaaaataaaaagagcatggataagatcatgaatt

SEQ ID NO: 48 - DNA, 565 bp, Oryza rufipogon
tctggtgaaaacagtgcatctttgcccagccaaaatgtaatcaatggtaattattctaggcagagttctgctacaatggccaagacaggacaggtcaattggactatgagcaacaacacaattagatcaatagaccctccacccaagtactcatcagaggaaagtccagcacttctgctagctagccacactaacaccgtggcagcgtactccaatcaaatcagtaaggatagtgatcggatctacaacttaccattcacaggtggtaatgggtcagatagtctacatgattggcatgttgatgagttctttagtaactcagaatttggctttgctgagcatggttcttctaagggcgacaacgctaagccaggtagtgctggtggatctccgcagtgccgtctggctgaaggcctgtttgttgaaggacttctaggtcaagtgcctgacaatccatggacagtgcctgaggttccctcgccaccgacagcctctggtctctattggcaaaataatttgctttgcccttcgtatgacagcaccatgttcgtccctgagatttcct

SEQ ID NO: 49 - DNA, 565 bp, Oryza rufipogon; identical to SEQ ID NO: 48

SEQ ID NO: 50 - DNA, 1449 bp, Oryza sativa; identical to SEQ ID NO: 47

SEQ ID NO: 51 - DNA, 1083 bp, Oryza sativa
atgaagatccagtgcaacgcgtgcggcgcggcggaggcgcgggtgctgtgctgcgccgacgaggcagcgctctgcacggcgtgcgacgaggaggtgcacgccgccaacaagctcgccgggaagcaccagcgggtgccgctgctctccgacgacggcggcgccgcgcccgccgccgccgccccggccgtgcccaagtgcgacatctgccaggaggcttctggatacttcttctgcctggaggaccgtgcacttctttgcagagattgtgatgtttctatacacacagtaaactcctttgtttcagtacaccaaagattcctactaacaggtgttcaagttggccttgatcctgctgatccagttccacctgttgctgacaagcatgttaagagtgctggtggttcagtggattcagcaactaaacatttgcaaaggaatcctacagacttatctggtgaaaacagtgcatctttgcccagccaaaatgtaatcaatggtaattattctaggcagagttctgttacaatggccaagacaggacaggtcaattggactatgagcaacaacacaattagatcaatagaccctccacccaagtattcatcagaggaaagtccagcacttctgctagctagccacactagcaccatggcagcgtactccagtcaaatcagtaaggatagtgatcggatctacaacttaccattcacaggtggtaatgggtcagatagtctacatgattggcatgttgatgagttctttagtaactcagaatttggctttgctgagcatggttcttctaagggtgacaacgctaagccagggagtgctggtggatctccgcagtgccgtctggctgaaggcctgtttgtcgaaggacttctaggtcaagtgcctgacaatccatggacagtgcctgaggtcccctcgccaccgacagcctctggtctctattggcaaaataatttgctttgcccttcgtacgacagcaccatgttcgtccctgagatttcctccttggagaactctcagaacaacttcactgtatctgctggtttgaagcgccgaaggaggcagttttga

SEQ ID NO: 52 - protein, 360 aa, Oryza sativa
MKIQCNACGAAEARVLCCADEAALCTACDEEVHAANKLAGKHQRVPLLSDDGGAAPAAAAPAVPKCDICQEASGYFFCLEDRALLCRDCDVSIHTVNSFVSVHQRFLLTGVQVGLDPADPVPPVADKHVKSAGGSVDSATKHLQRNPTDLSGENSASLPSQNVINGNYSRQSSVTMAKTGQVNWTMSNNTIRSIDPPPKYSSEESPALLLASHTSTMAAYSSQISKDSDRIYNLPFTGGNGSDSLHDWHVDEFFSNSEFGFAEHGSSKGDNAKPGSAGGSPQCRLAEGLFVEGLLGQVPDNPWTVPEVPSPPTASGLYWQNNLLCPSYDSTMFVPEISSLENSQNNFTVSAGLKRRRRQF

SEQ ID NO: 53 - DNA, 5810 bp, Oryza sativa
tttgctgatgtgctcgcctgcgctttgaccaccgcttcgtccccttctacagcctctcgctaagctaattatcaccattcaaagcaagcgaagccgctaaaccacacctactaaccacgcaattagacaaaacaaatcacactcgcttcgctaatcgctattcgctaactaaccaccacctccgcttaaacaaaaaaaaaagattaataaagcagtggggatttgggaagaaagggaggtatacagtggagagataggggaagagagggagcgatccaatcgcgtcgctaaaaccctagcggctggctgatttgggggattgctcgtcggagagatgaagatccagtgcaacgcgtgcggcgcggcggaggcgcgggtgctgtgctgcgccgacgaggcagcgctctgcacggcgtgcgacgaggaggtgcacgccgccaacaagctcgccgggaagcaccagcgggtgccgctgctctccgacgacggcggcgccgcgcccgccgccgccgccccggccgtgcccaagtgcgacatctgccaggtcgcccccctcccctcccctcccctcctcgctctttctgcctcgattagggtagttgaggcgtgcaggaaattttgcctcgatttgtttgatagtagttttggatgttcttaccgagtatagtcagttagcaaagcgaaattactacgtttatcagcgattagaagaagctattcttccatgttgatcaagctgtgctggcacacgtttatcagcgattagaagaagctactcttccatgttgatcaagctgtgccggcactggagagggggtagtaactagtaagcatatggaagcacaaataggaattcctgtatatcctgagctaaaaattgagggggttgctattgatactatgatgaacatctataagtaaaaccccatttcgccgaggtgttttcttatcttgtgttgtatgcttaagcttcttaatcacaattagccaatttcacatctagttaaccactatatattttttatcagtggaaggatcaaactctctttaaggatacaaactgaattctcacagacttggatagtatgaggtaaaaaggaggttagaaaagattgacagttcatagttatggtttatattgtgcttgatgaatgaacactagagtgaagagacaagtctggtaatgatcttttctgttggtgctgtgtgaaagtatacggtccctaatatgatatcaaatgagatgatataaaataaacggtgaagtgatgactatctggtaaccctagctaaagctatgctttgccatttcaatcatctaaactagggcaaatatttgaacattacagttgcataggtttcttacactgaggacacatattgtaaagtagcaccgtaccaacatttaatagagtgatgtcatgctgcacattaagaccagctttttttttttccgatgctctgaggtagactgtaagccttgtgcagttttacatagcaaatcctagtatgtagtgactgtatgtgttttataggcttatcgggtcatttctagttgacagattgttatatgtaaaacttattgtttggaatacctaaaaaaaaataaggaaaaagtatatgctatttgctttgcatgtttatttgtgctgcactttaagtccaagatcttgcaaatgttttcttggttttttgcatatcaacgtgattatgcggtaagaaaattaaattttgaggcctctagcaaaaacatagaaagtcattggttggatagatacttatttatagtttatctgaaccccatcctttatttccataaggacgtttgtccaactgtattgttctattttagacggtgagatatggtaccagactggataggttggatgtagatcttttatgatgacatgatgaaatatggcactgttcagttggctgtgtcatgtgcaactgtgtaaagtccatgtgcaaagaaagggacaactaatggtctgattattggacacatgactgaagtgaacccacataggtttcattgcatctcgttggttcctgctggaactgcaagttgtgctatttatccttgatttattaggcgcagatctcaaactttatttgtcattgcaggaggcttctggatacttcttctgcctggaggaccgtgcacttctttgcagagattgtgatgtttctatacacacagtaaactcctttgtttcagtacaccaaagattcctactaacaggtgttcaagttggccttgatcctgctgatccagttccacctgttgctgacaagcatgttaagagtgctggtggttcagtggattcagcaactaaacatttgcaaaggaatcctacagacttatctggtgaaaacagtgcatctttgcccagccaaaatgtaatcaatggtaattattctaggcagagttctgttacaatggccaagacaggacaggtcaattggactatgagcaacaacacaattagatcaatagaccctccacccaagtattcatcagaggaaagtccagcacttctgctagctagccacactagcaccatggcagcgtactccagtcaaatcagtaaggatagtgatcggatctacaacttaccattcacaggtggtaatgggtcagatagtctacatgattggcatgttgatgagttctttagtaactcagaatttggctttgctgagcatggttcttctaaggtattccaaaatattatatatatgtaaattgctggttgcctgtttttcctgttatcaacagtatgtcgtgcttatttagtaattttttggaatgcagagtttaagcatgatactttgattaatattagagtactgatacaggacatgcacacactctctctctcacactctctcacacacacagacaaatacctgtcgaattgttagttgttcagtgaatgcatttaaaattaggagtttttactgtgatacaattagacttctgttataatttagtttattctagatgtttagatgttaacctttccgtcattcgttaagtgaaaacctgtacctaagcttatctgtgtttatgattaatgagaatttcttacctagttgctccttccaattgatatatgctgatggaactgtgcaatgctcaagtcctagaatcaatctagcatgctgatgctttgggggtttggattctatgacaatcttttgctgaaatataaattgtttaccgtaactatcaagcttataagacagtaacttaaagtctgtggaagtgattgctgttcccttggcgtatgctgtttcatgtgtgcttttggatgaattccaccacctttgctatatacagttcattgcgactaattttagagctgtcaatcatgatttttctagcatgtcttgttcgattatgcaatgtgactgaaccatactccctctgtcccaaaaaaaggcaacctagtactaggatgtgtcacgtcgtagtacgaggttgagtttttttgggacggagggagtactgctgacagaatgagggctagatttttgtgtggcatttacatcgatgcttaatttatttgcaagtagactttctattaccaaagaagctcactattgaatatctttctcttctcctgctgggttgaatttctccatttaggtttgcttcttatgccgttgttgttttggctgtgcacttacagggtgacaacgctaagccagggagtgctggtggatctccgcagtgccgtctggctgaaggcctgtttgtcgaaggacttctaggtcaagtgcctgacaatccatggacagtgcctgaggtcccctcgccaccgacagcctctggtctctattggcaaaataatttgctttgcccttcgtacgacagcaccatgttcgtccctgagatttcctccttggagaactctcagaacaacttcactgtatctgctggtttgaagcgccgaaggaggcagttttgatctgccgatagcatcagacaccattcggtttagttggcaggtcacgttgagaggagggcttcttgcctgcctgaccaaagccacaaaacacagggatggatgatcatccagtggtcagtctaaaaatatgttgtaatgtttgtgtttcgactatacgtttgttgctgaagtttaaaggcgagggaattgttgtgagatctgggatgacagatttgttgttaccaattttgtgaatagtgaaaataaaaagagcatggataagatcatgaattcatgatgcgcaggttaatacatgttgaagttccttagccacgaacagttttggtagcggggtgtatgatgtatgatgttaaccacaatttttcgagctagtttatggagctagacgacctgggttcaaacctcttctcttctaattatttgatattaggtcatttcttaatattcgtggttttttatgtgacgccaggtattgcaagcttgggatttatcctaacgtctcactgtatttggaaatttcatatgaattttacagagatttgtaagaatttgataaaaacacacacttcaatttcagtatgagacgggagaagttcagttcagttcacacgttcgtagggatttgatggttttgcttttgcatcaccaccactcaccagttctcttctgtaccacgaaggataaaatcagagccgtccatccaggtatccgacggttcagatactcccaacgtgttgaccccttcggccatttgcgactgccgctttgcagaagcatcacggccgcatcggcgaccccgaactccgccgccgcccgcagccgccgccgccttcgcagccgccgccgccgccgccttcttccagggcaaacgatgctccgccgcgtcgcgccatccctgcggcgagctatcctcacatcctccgcccatggccgcgcgggcgcgcaactcacggagcctggtctctcgcctcgccacgctctgctcccgcaatggcgcctttgctcctccgcggcctccacgaactctcccccgcccccgccgccgcctccttcttctccgcctcagggaacaccgcgtccagcaggcggatccaccgtctcgtcgctgaaccctgccgaggtcgccaagttcgccgccatcgctgagacctggtgagatcccccctccactccctcccattcctggcatggttttttgagatgatgaactaccacgccctcgcgatgcttggccaatttcgtttagtgcaatgacattgatagtgctttagtagtaattttgtacttggtgaattgtggtgaatactgcttctagcatgagctcagtcaacaaatttgattgtactctttgttttttggcactcgaatagtagaattctcatttaggagcagattcacaggaaaaaaaaagtggggttacatatgtgttgtttcaaatggaatgaacctccgacttatcacctgagcatatgattgcttattagtggcacaattcctgcaataaacaggtgggattctgaaggtccattcaagccgttgcatctgatgaatccaactcgactgtcttttatccggtccactctctgtagacatttcaggtacagttcctcagtcattgtattcatttgtggttgtgattatcactggtaaacctgaataagtaaagacaggacatagtgtcaactcttgttttgccccctaaccatgtgtgttcactctgctaggcgggatccaaattcttctaaaccacttgaaggccttaaagttattgatgttggatgtgggggtggcattctctcagaggtaccatcatttttttcttataatctgcta

SEQ ID NO: 54 - DNA, 22 bp, Oryza sativa
ttcagttggctgtgtcatgtgc

SEQ ID NO: 55 - DNA, 28 bp, Oryza sativa
ggtacaggttttcacttaacgaatgacg

SEQ ID NO: 56 - DNA, 903 bp, Hordeum vulgare
cgccgcgccgcccaagtgcgacatctgccaggaggcttctggatacttcttctgcctggaggaccgtgctctactttgtagagattgtgatgttgctatacacacagtgaattcctttgtttcagcacatcaaaggttcctgctgacaggagttcaagttggcctcgatcctgccgatcctgttccacctgttactgaaaagcatgttaacacttctagtggatcagtggattcacaaccaaaacacctgccaaagaaaaatcctacagtcctggtttcaggtgaaatcggtggatctattcccagccaaaatgcaatcagtgaggattattctaggcagatttctgctccaaataccaagaccggaatggccaattggactatcgacaacagtgcacttagattggcagagcctccacccaagtacctgtcagatggaaattcaaaacttatgctctctggtcagaccaccgcaggtctatccaaccaaatgaacagagatactggccgagcttacaacttacctttctcaggcggtaatgggtcagacagtttacctgattggcctgtggacgaattcttcagtaacttagaatatggtccaaactttggcttcactgagcatggttcttccaagggtgacaatgctaagctagggagtgctgggggatcaccacagtgccgcctagctgaaggcctgtttgcagaagatctgcttggccaggtacctggatttgatgctgaggatccatgggtggtgcccgaggttccctccccaccaacagcctcggctctgtgctggcaagggaatttgcattaccctgtgtatgacggcgccatgttcgtccctgaagctccctccctgcagagctctcaggaccacttaactgcatctgccggttt

SEQ ID NO: 57 - DNA, 903 bp, Hordeum vulgare; identical to SEQ ID NO: 56

SEQ ID NO: 58 - DNA, 1620 bp, Zea mays
ccatcgtggtctggcctcgtcgccgacggacgaccgacgcgcacccccgccatgctctccagccgaagatgcttcgcggcggcggccaaccgggtccggccgctcgccagggccttctgcgacgcgccggcctcgcgcgcggatgcgggggcctccgggacgcccagcccgcaggatcacgccgagaaggttgctgccctgaaggatccgcccgatgtgctagacgttgctatcgttggtggaggcatggtgggcttggctgtcgcttgtgcattgtcaaatatgccgttaacgaaacaccttagagtcgctattatcgatagcaatccagcgctgaagtcaaggagttacctggataaaaacagtatacccgattctagggtcagcaccgttacccctgcaaccatttctttcttcaaggatattggcgcctgggagcatgttcaacagcaaagacatgctttctttggtaaaatgcaggtgtgggattacactggacttggatacacgaggtataatgctagagatgtgggcaaagaacaccttggatgtgtggtggagaacaaggtgctttgcaactcactgctcttacgtttacaggaagaattggatgatattgaaaatgtgatatatcctaccagattggtgtctcttactttcccgttgaagaccagacaagcaggaatgaaaccaacatcaagagaaccattatctgttggtcatactacagaagagttccatcgcagtcatttagttaaacttgatctcagtgatggacggagtttatattccaaattggtgataggagctgatggttcgaagtccaatgttagacagattgcaggcataaaaacaactagttggaattatccgcagagtgcaattatctgtacagtagagcatgttgtggaaaatgattgtgcgtggcagaggtttctcccttctggtccaattgcgctacttccgataggcaataacttcagcaacattgtatggacaatgagtccagaggagtcactgcgccataagtcaatgagcccagaagagtttgtgaattctgtgaaccacgcattggattttggttatggtccgcatccacactctaccgctcttgaccattacatggaacagttcttttctggcattgggagcagtgcagcatctacaaaggaatgcttcgaagtgccaccgaaagcaacccgtgtagtttttgagagaatggcgtttccattgtcgttgatgcattcccatgattatgtttccaaaaggttagcattagttggagatgctgctcatactgtccaccccttagccggccaaggtgtaaacttgggctttggtgatgctgctgctttagcaaaagttattgctgagggagtttcagtgggctcggatgttggggatctgactttgctacagcggtatgagaaggagaggaaggctgccaatgtggcgatggcagcagtgctagatggttttcagaagatgtactctgttgatttggggccccttaatgtagtgagagctgctgcgttccacggtgcccagtacatatcaccactaaaaaagaacatcatyttttatgcaacgggtgacacgaaatggtcactatttcagtga

SEQ ID NO: 59 - DNA, 1109 bp, Zea mays
ctgcctagaggaccgtgcactactttgtagagactgtgatgttgctatacacacagtgaattcctttgtttcagtacatcagagattcctgctaactggagttcaggtgggtcttgatcctgctgatccagttccacctattgctgaaaagcatgttaatgctgctggtggatcagtaaatcaaccagtgagacatcagccaaggagaagcccgacagttcaattttcagttgaagggagtgcttctgttcctaccaaaaatgtaacaaatggagattgttcaaggcagaattttgtcccaacagccagggcagaagtggttgattggactatgaataatagtacaattcgatcagtagaatctccgcctaagtacatttcagaggaaagccccacacttctgcaatctagccagaccacaacagtcttctccaaccaaattaatggcaatagtgatggggcctaccacttgtcattctcaggtggtaatgtgacagacagcctaccagattggcccgtggatgagttcttcagtaactcagaatatggtccaaactttggcttctccgagaatggttcttctaagggtgataccgctaagctggggggcgctggtggatctccacaatgccgtttggctgaaggctcagttgctgaggaactattagggcaggtgcctggattgatcaccgatgaatatatgggccgagtacctgagaattcatggacagtgcctgaggttccctctccaccaacagcctcggggctcaactggcatgggaatttgtgtttccctgcgtatgacagcaccatgtttgttcctgaaattacctccctccagaactcccaaagtcacttcaccgtaccttccagtttcaagcgccggagaagagagtactaagaggcgacaagaagatcactagacaccactgtccggtctaggccatcttgagaaaggcttcttggtgggccaaatgacgaaagaaaatatccaggactgagtggtcattcaaacatcaactgagctatccaagtagccaggttttaagtgtgttgtaatgcttcaatgtgtttcattttctaaagacctgagcttcgcttgtgtttga

SEQ ID NO: 60 - DNA, 1001 bp, Zea mays
acaggatagtccaacacttctgcaatctattcagaccacaacactcttctctaaccaaattaacagcgatagtgatgggacctaccacttgtcattctcaagtggtaatgtgacagacagtctaccacattggcctgtggatgagttcttcactaactcacaatatgttccaaacttacgcttctccgagcatggttcttctaagggtgataacgctaagctggagagtgctggtggatctctacaatgccgtttagctgaaggctccattgctgaggaactattaggacaggtgcctgggttgatcactgatgattatatgagccgggtacctgagacttcatggacagtacctgaggttccctcgccaccaactgcctcggggctcaattggcatgggaatttgtgtttccctgcatatgacagcacaatgtttgttcctgaaattacatccctccagaactcccaaaatcagttcaccgtaccttcgagtttcaagcgccggagaagagagtattgaggtgacgacaagatcactagacatccactgtccagtttaggccatcttgagaaaggtttcttgctgtaccaaatgacggagaaaggatatccaggactgggttatcattcaaacttcaacagaacaatccaagtagctaggtttgaagtgtgctgcaatgcttcaatgatgttcattttccaaagatctaagcttcgctagtsttggggtcttcttstccscgaaggccctttagacaaaaacmccttcggcaagatagtacaaaggcaggtgcaacatgaagatataagccsaagctacaatcgaaagagcttcggtttggtgtcatgctaaagacgaaggacgataccgacttacaagaggaaaatacggtatagcccttggtagtccgtattacgatcatacttagttattactgatataaatgtaatttcgttgggggtacggctcgcgcctataaatcgatgaccaatatcctcct

(19) United States
(12) Patent Application Publication
(10) Pub. No.: US 2011/0173723 A1
(43) Pub. Date: Jul. 14, 2011

(54) EG82013 and EG81345 Nucleic Acids and Uses Thereof

(75) Inventor: Walter Messier, Longmont, CO (US)

(73) Assignee: EVOLUTIONARY GENOMICS, INC., Lafayette, CO (US)

(21) Appl. No.: 13/056,562

(22) PCT Filed: Jul. 29, 2009
(86) PCT No.: PCT/US09/52143

§ 371 Date: Mar. 11, 2011

Related U.S. Application Data
(60) Provisional application No. 61/084,457, filed on Jul. 29, 2008.

(51) Int. Cl. A01H 5/00; C07H 21/04; C07K 14/415; C12N 5/10; C12N 15/63; C12Q 1/68

(52) U.S. Cl. 800/295; 536/23.6; 530/372; 435/419; 435/320.1; 435/6.11

(57) ABSTRACT

The present invention provides methods for identifying nucleic acid and polypeptide sequences which may be associated with a commercially relevant trait in plants, specifically, so-identified nucleic acids and polypeptide sequences for yield genes EG82013 and EG81345. Sequences thus identified are useful in enhancing commercially desired traits in domesticated plants or wild ancestor plants, identifying related nucleic acid sequences, genotyping a plant, and marker assisted breeding. Sequences thus identified may also be used to generate heterologous DNA, transgenic plants, and transfected host cells.

### FIELD OF THE INVENTION

The invention relates to molecular and evolutionary techniques to identify nucleic acid and polypeptide sequences corresponding to commercially relevant traits, such as yield, in ancestral and domesticated plants, the identified nucleic acid and polypeptide sequences, and methods of using the identified nucleic acid and polypeptide sequences.

### BACKGROUND OF THE INVENTION

Humans have bred plants and animals for thousands of years, selecting for certain commercially valuable and/or aesthetic traits. Domesticated plants differ from their wild ancestor or family members in such traits as yield, short day length flowering, protein and/or oil content, ease of harvest, taste, disease resistance and drought resistance. Domesticated animals differ from their wild ancestor or family members in such traits as fat and/or protein content, milk production, docility, fecundity and time to maturity. At the present time, most genes underlying the above differences are not known, nor, as importantly, are the specific changes that have evolved in these genes to provide these capabilities. Understanding the basis of these differences between domesticated plants and animals and their wild ancestor or family members will provide useful information for maintaining and enhancing those traits. In the case of crop plants, identification of the specific genes that control desired traits will allow direct and rapid improvement in a manner not previously possible.

The identification in domesticated species of genes that have evolved to confer unique, enhanced or altered functions compared to homologous ancestral genes could be used to develop agents to modulate these functions. The identification of the underlying domesticated species genes and the specific nucleotide changes that have evolved, and the further characterization of the physical and biochemical changes in the proteins encoded by these evolved genes, could provide valuable information on the mechanisms underlying the desired trait. This valuable information could be applied to DNA marker assisted breeding or DNA marker assisted selection. Alternatively, this information could be used in developing agents that further enhance the function of the target proteins. Alternatively, further engineering of the responsible genes could modify or augment the desired trait. Additionally, the identified genes may be found to play a role in controlling traits of interest in other domesticated plants or family members.

Humans, through artificial selection, have provided intense selection pressures on crop plants. This pressure is reflected in evolutionarily significant changes between homologous genes of domesticated organisms and their wild ancestor or family members. It has been found that only a few genes, e.g., 10-15 per species, control traits of commercial interest in domesticated crop plants. These few genes have been exceedingly difficult to identify through standard methods of plant molecular biology.

Methods for identifying genes changed due to domestication are described in related patents and applications listed above. Methods for DNA marker assisted breeding (MAB) and DNA marker assisted selection (MAS) are well known to those skilled in the art and have been described in many publications (see for example Peleman and van der Voort, Breeding by Design, TRENDS in Plant Science 8(7):330-334). Such methods can make plant breeding more efficient by increasing the ability to select and incorporate specific alleles associated with a desired phenotype during the development of new plant varieties. One problem with markers generally used today is that they can become separated from target genes or traits through recombination (see Holland in Proceedings of the 4^{th} International Crop Science Congress 26 Sep.-1 Oct. 2004, Brisbane, Australia). In fact, Holland cites examples where use of markers was better than conventional breeding, and other examples where conventional breeding gave better results than marker assisted breeding. Holland states that “it is not likely that markers will soon be generally useful for manipulating complex traits like yield”. What is needed for markers to be useful for manipulating complex traits like yield are the specific genes underlying such complex traits instead of markers that are only sometimes associated with such complex traits.

### SUMMARY OF THE INVENTION

In one embodiment, the present invention includes a method for identifying an EG81345 homolog nucleic acid sequence or an EG81345 allele in a plant. This method includes the following steps. In one step, at least a portion of the plant nucleic acid sequence is compared with at least one nucleic acid. This nucleic acid can be any of the following: an isolated nucleic acid comprising at least 20 contiguous nucleotides of a nucleic acid selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:59, and SEQ ID NO:60; a nucleic acid having at least about 80% sequence identity to a foregoing nucleic acid and is a marker for yield or a yield gene in a plant; and the complement of any of the foregoing nucleic acids. The method also includes identifying at least one nucleic acid sequence that is identical to any of the foregoing nucleic acids in the plant. This method may also be carried out with EG81345 polypeptides. Alternatively, the method comprises comparing one of the above-named SEQ ID NOs, or at least a 20 nucleotide portion thereof, or a complement thereof, and identifying at least one nucleic acid sequence having at least about 80% sequence identity thereto.

In another embodiment, the present invention includes a method for identifying an EG82013 homolog nucleic acid sequence or an EG82013 allele in a plant. This method includes the following steps. In one step, at least a portion of the plant nucleic acid sequence is compared with at least one nucleic acid. This nucleic acid can by any of the following: an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:58 and SEQ ID 61; and a nucleic acid having at least about 80% sequence identity to a foregoing nucleic acid and is a marker for yield or a yield gene in a plant; and the complement of any of the foregoing nucleic acids. The method also includes identifying at least one nucleic acid sequence that is identical to any of the foregoing nucleic acids in the plant. This method may also be carried out with EG82013 polypeptides. Alternatively, the method comprises comparing one of the above-named SEQ ID NOs, or at least a 20 nucleotide portion thereof, or a complement thereof, and identifying at least one nucleic acid sequence having at least about 80% sequence identity thereto.

In another embodiment, the invention includes methods for marker assisted breeding including a method of marker assisted breeding of plants for a particular EG81345 nucleic acid sequence. This embodiment includes the following steps. One step includes comparing, for at least one plant, at least a portion of the nucleotide sequence of said plants with a particular EG81345 nucleic acid sequence of the present invention, such as, for example, at least a portion of those selected from the group consisting of (i) a nucleic acid comprising a nucleic acid selected from the group consisting of an isolated nucleic acid comprising at least 20 contiguous nucleotides of a nucleic acid selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:59, and SEQ ID NO:60; a nucleic acid having at least about 80% sequence identity to a foregoing nucleic acid and is a marker for yield in a plant; and the complement of any of the foregoing nucleic acids. This method also includes the step of identifying whether the plant comprises the particular nucleic acid sequence; and the step of breeding a plant comprising the particular nucleic acid sequence to produce progeny. This method may also be carried out with EG81345 polypeptides. Alternatively, the method comprises comparing one of the above-named SEQ ID NOs, or at least a 20 nucleotide portion thereof, or a complement thereof, and identifying at least one nucleic acid sequence having at least about 80% sequence identity thereto.

Methods for marker assisted breeding also include a method of marker assisted breeding of plants for a particular EG82013 nucleic acid sequence. Steps include comparing, for at least one plant, at least a portion of the nucleotide sequence of said plants with a particular EG82013 nucleic acid sequence of the present invention, such as, for example, at least a portion of a nucleic acid sequence selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:58; a nucleic acid having at least about 80% sequence identity to a foregoing nucleic acid and is a marker for yield in a plant; and the complement of any of the foregoing nucleic acids; identifying whether the plant comprises the particular nucleic acid sequence; and breeding a plant comprising the particular nucleic acid sequence to produce progeny. This method may also be carried out with EG82013 polypeptides. Alternatively, the method comprises comparing one of the above-named SEQ ID NOs, or at least a 20 nucleotide portion thereof, or a complement thereof, and identifying at least one nucleic acid sequence having at least about 80% sequence identity thereto.

In one embodiment, the present invention includes EG81345 and EG82013 nucleic acids which include an isolated nucleic acid comprising a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59; and SEQ ID NO:60; a nucleic acid having at least about 80% sequence identity to a foregoing nucleic acid and is a marker for yield or a yield gene in a plant. Isolated nucleic acids also includes complements of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59; and SEQ ID NO:60.

The present invention also includes an isolated polypeptide which comprises (includes) at least a 6 amino acid portion of SEQ ID NO:5 or SEQ ID NO:52; at least a portion of one or more of a polypeptide encoded by a nucleic acid comprising a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59; and SEQ ID NO:60; a nucleic acid having at least about 80% sequence identity to a foregoing nucleic acid and is a marker for yield or a yield gene in a plant; and the complement of any of the foregoing nucleic acids; and a polypeptide encoded by a nucleic acid having at least about 80% sequence identity to a nucleic acid enumerated above and confers substantially the same yield as a nucleic acid enumerated above.

In another embodiment, the present invention includes a method for identifying one or more alleles of the gene encoding EG81345 in a plant. In particular, this method comprises: assaying a sample of nucleic acids from a plant for the presence of one or more single nucleotide polymorphisms in the plant EG81345 gene, wherein single nucleotide polymorphisms are selected from the group consisting of: SEQ ID NO:51, position 521, C or T; SEQ ID NO:51, position 600, C or T; SEQ ID NO:51, position 644, A or G; SEQ ID NO:51, position 649, G or A; SEQ ID NO:51, position 665, A or G; SEQ ID NO:51, position 807, C or T; SEQ ID NO:51, position 825, T or G; SEQ ID NO:51, position 873, T or C; SEQ ID NO:51, position 924, T or C; and SEQ ID NO:51, position 984, T or C. The equivalent nucleotide positions on the disclosed O. rufipogon sequence herein are SEQ ID NO:49, position 71, C or T; SEQ ID NO:49, position 150, C or T; SEQ ID NO:49, position 194, A or G; SEQ ID NO:49, position 199, G or A; SEQ ID NO:49, position 215, A or G; SEQ ID NO:49, position 357, C or T; SEQ ID NO:49, position 375, T or G; SEQ ID NO:49, position 423, T or C; SEQ ID NO:49, position 474, T or C; and SEQ ID NO:49, position 984, T or C.

The present invention also includes a method for identifying one or more alleles of the gene encoding EG82013 in a plant, comprising: assaying a sample of nucleic acids from a plant for the presence of one or more single nucleotide polymorphisms in the plant EG82013 gene, wherein single nucleotide polymorphisms are selected from the group consisting of: SEQ ID NO:2 (or SEQ ID NO:4), position 72, C or T; SEQ ID NO:2 (or SEQ ID NO:4), position 78, C or T; SEQ ID NO:2 (or SEQ ID NO:4), position 132, A or G; SEQ ID NO:2 (or SEQ ID NO:4), position 183, C or T; SEQ ID NO:2 (or SEQ ID NO:4), position 279, A or G; SEQ ID NO:2 (or SEQ ID NO:4), position 306, C or T; SEQ ID NO:2 (or SEQ ID NO:4), position 340, T or C; SEQ ID NO:2 (or SEQ ID NO:4), position 348, G or A; SEQ ID NO:2 (or SEQ ID NO:4), position 414, A or G; SEQ ID NO:2 (or SEQ ID NO:4), position 546, A or G; SEQ ID NO:2 (or SEQ ID NO:4), position 555, T or C; SEQ ID NO:2 (or SEQ ID NO:4), position 690, T to G; SEQ ID NO:2 (or SEQ ID NO:4), position 851, A or G; SEQ ID NO:2 (or SEQ ID NO:4), position 901, A or G; SEQ ID NO:2 (or SEQ ID NO:4), Position 924, G to A; SEQ ID NO:2 (or SEQ ID NO:4), position 953, C or G; SEQ ID NO:2 (or SEQ ID NO:4), position 1000, G or A; SEQ ID NO:2 (or SEQ ID NO:4), position 1012, A or T; SEQ ID NO:2 (or SEQ ID NO:4), position 1016, T or A; SEQ ID NO:2 (or SEQ ID NO:4), position 1153, A or T; SEQ ID NO:2 (or SEQ ID NO:4), position 1174, G or A; SEQ ID NO:2 (or SEQ ID NO:4), position 1185, T or A; and SEQ ID NO:2 (or SEQ ID NO:4), position 1248, T or A.

One embodiment of the present invention includes a method of making a transfected plant cell or a transgenic plant comprising transfecting a plant cell with an EG82013 or EG81345 polynucleotide.

### DETAILED DESCRIPTION OF THE INVENTION

With the present invention, the inventors have identified genes, nucleic acids, and polypeptides corresponding to EG82013 (for O. sativa (domesticated rice) and O. rufipogon (ancestral rice)), and nucleic acids corresponding to EG81345 (for O. sativa (domesticated rice) and O. rufipogon (ancestral rice). The inventors also have selected ESTs from publicly available sources that correspond to the aforementioned genes in several other plant species. These genes have both been correlated with yield and have been shown to control yield by the inventors. The nucleic acids and polypeptides of the present invention are useful in a variety of methods such as a method to identify a nucleic acid sequence that is associated with yield or is a marker for yield in a plant; a method of determining whether a plant has one or more of a nucleic acid sequence comprising an EG81345 or EG82013 sequence; and a method for marker assisted breeding of plants for a particular EG81345 or EG82013 sequence. The nucleic acids and polypeptides of the present invention are also useful for creating plant cells, propagation materials, transgenic plants, and transfected host cells. More specifically, the nucleic acids and polypeptides of the present invention may be used as markers for improved marker assisted selection or marker assisted breeding. Moreover, such nucleic acids and polypeptides can be used to identify homologous genes in other species that share a common ancestor or family member, for use as markers in breeding such other species. For example, maize, rice, wheat, millet, sorghum and other cereals share a common ancestor or family member, and genes identified in rice can lead directly to homologous genes in these other grasses. Likewise, tomatoes and potatoes share a common ancestor or family member, and genes identified in tomatoes by the subject method are expected to have homologues in potatoes, and vice versa.

The practice of the present invention employs, unless otherwise indicated, conventional techniques of molecular biology, genetics and molecular evolution, which are within the skill of the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Current Protocols in Molecular Biology” (F. M. Ausubel et al., eds., 1987); “PCR: The Polymerase Chain Reaction”, (Mullis et al., eds., 1994); “Molecular Evolution”, (Li, 1997).

It is to be noted that the term “a” or “an” entity refers to one or more of that entity; for example, a gene refers to one or more genes or at least one gene. As such, the terms “a” (or “an”), “one or more” and “at least one” can be used interchangeably herein. It is also to be noted that the terms “comprising,” “including,” and “having” can be used interchangeably.

As used herein, a “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, a “gene” refers to a nucleic acid or portion of a nucleic acid comprising a sequence that encodes a protein. It is well understood in the art that a gene also comprises non-coding sequences, such as 5′ and 3′ flanking sequences (such as promoters, enhancers, repressors, and other regulatory sequences) as well as introns.

The terms “polypeptide,” “peptide,” and “protein” are used interchangeably herein to refer to polymers of amino acids of any length. These terms also include proteins that are post-translationally modified through reactions that include glycosylation, acetylation and phosphorylation.

The term “domesticated organism” refers to an individual living organism or population of same, a species, subspecies, variety, cultivar or strain that has been subjected to artificial selection pressure and developed a commercially or aesthetically relevant trait. In some preferred embodiments, the domesticated organism is a plant selected from the group consisting of maize, wheat, rice, sorghum, tomato or potato, or any other domesticated plant of commercial interest, where an ancestor or family member is known. A “plant” is any plant at any stage of development, particularly a seed plant.

The term “wild ancestor or family member” or “ancestor or family member” or “ancestor/family member” means a forerunner or predecessor organism, species, subspecies, variety, cultivar or strain from which a domesticated organism, species, subspecies, variety, cultivar or strain has evolved. A domesticated organism can have one or more than one ancestor or family member. Typically, domesticated plants can have one or a plurality of ancestor or family members. The term “family member” means a member of the same taxonomic family as a species. For example, rice and corn are in the Grass taxonomic family. Other “family members” in the Grass family include switchgrass, sugar cane, sorghum, miscanthus, and others.

The term “commercially or aesthetically relevant trait” is used herein to refer to traits that exist in domesticated organisms such as plants or animals whose analysis could provide information (e.g., physical or biochemical data) relevant to the development of improved organisms or of agents that can modulate the polypeptide responsible for the trait, or the respective nucleic acid. The commercially or aesthetically relevant trait can be unique, enhanced or altered relative to the ancestor or family member. By “altered,” it is meant that the relevant trait differs qualitatively or quantitatively from traits observed in the ancestor or family member. A preferred commercially or aesthetically relevant trait is yield.

The term “K_{A}/K_{S}-type methods” means methods that evaluate differences, frequently (but not always) shown as a ratio, between the number of nonsynonymous substitutions and synonymous substitutions in homologous genes (including the more rigorous methods that determine non-synonymous and synonymous sites). These methods are designated using several systems of nomenclature, including but not limited to K_{A}/K_{S}, d_{N}/d_{S}, D_{N}/D_{S}.

The terms “evolutionarily significant change” and “adaptive evolutionary change” refer to one or more nucleotide or peptide sequence change(s) between two organisms, species, subspecies, varieties, cultivars and/or strains that may be attributed to either relaxation of selective pressure or positive selective pressure. One method for determining the presence of an evolutionarily significant change is to apply a K_{A}/K_{S}-type analytical method, such as to measure a K_{A}/K_{S} ratio. Typically, a K_{A}/K_{S} ratio of 1.0 or greater is considered to be an evolutionarily significant change.

Strictly speaking, K_{A}/K_{S} ratios of exactly 1.0 are indicative of relaxation of selective pressure (neutral evolution), and K_{A}/K_{S} ratios greater than 1.0 are indicative of positive selection. However, it is commonly accepted that the ESTs in GenBank and other public databases often suffer from some degree of sequencing error, and even a few incorrect nucleotides can influence K_{A}/K_{S} ratios. For this reason, nucleic acids with K_{A}/K_{S} ratios as low as 0.75 can be carefully resequenced and re-evaluated for relaxation of selective pressure (neutral evolutionarily significant change), positive selection pressure (positive evolutionarily significant change), or negative selective pressure (evolutionarily conservative change).

The term “positive evolutionarily significant change” means an evolutionarily significant change in a particular organism, species, subspecies, variety, cultivar or strain that results in an adaptive change that is positive as compared to other related organisms. An example of a positive evolutionarily significant change is a change that has resulted in enhanced yield in crop plants. As stated above, positive selection is indicated by a K_{A}/K_{S} ratio greater than 0.75; positive selection is also seen with higher ratios, such as those greater than 1.0. In other embodiments, the K_{A}/K_{S} value is greater than 1.25, 1.5 and 2.0.

The term “neutral evolutionarily significant change” refers to a nucleic acid or polypeptide change that appears in a domesticated organism relative to its ancestral organism, and which has developed under neutral conditions. A neutral evolutionary change is evidenced by a K_{A}/K_{S} value of between about 0.75-1.25, preferably between about 0.9 and 1.1, and most preferably equal to about 1.0. Also, in the case of neutral evolution, there is no “directionality” to be inferred. The gene is free to accumulate changes without constraint, so both the ancestral and domesticated versions are changing with respect to one another.

The term “homologous” or “homologue” or “ortholog” is known and well understood in the art and refers to related sequences that share a common ancestor or family member and is determined based on degree of sequence identity. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to, (a) degree of sequence identity; (b) same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least about 99%.

Homology can be determined using software programs readily available in the art, such as those discussed in Current Protocols in Molecular Biology (F. M. Ausubel et al., eds., 1987) Supplement 30, section 7.718, Table 7.71. Preferred alignment programs are MacVector (Oxford Molecular Ltd, Oxford, U.K.) and ALIGN Plus (Scientific and Educational Software, Pennsylvania). Another preferred alignment program is Sequencher (Gene Codes, Ann Arbor, Mich.), using default parameters. An expanded discussion can be found below.

The term “nucleotide change” refers to nucleotide substitution, deletion, and/or insertion, as is well understood in the art.

“Housekeeping genes” is a term well understood in the art and means those genes associated with general cell function, including but not limited to growth, division, stasis, metabolism, and/or death. “Housekeeping” genes generally perform functions found in more than one cell type. In contrast, cell-specific genes generally perform functions in a particular cell type and/or class.

The term “agent”, as used herein, means a biological or chemical compound such as a simple or complex organic or inorganic molecule, a peptide, a protein or an oligonucleotide that modulates the function of a nucleic acid or polypeptide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

The term “to modulate function” of a nucleic acid or a polypeptide means that the function of the nucleic acid or polypeptide is altered when compared to not adding an agent. Modulation may occur on any level that affects function. A nucleic acid or polypeptide function may be direct or indirect, and measured directly or indirectly.

A “function of a nucleic acid” includes, but is not limited to, replication; translation; expression pattern(s). A nucleic acid function also includes functions associated with a polypeptide encoded within the nucleic acid. For example, an agent which acts on a nucleic acid and affects protein expression, conformation, folding (or other physical characteristics), binding to other moieties (such as ligands), activity (or other functional characteristics), regulation and/or other aspects of protein structure or function is considered to have modulated nucleic acid function.

A “function of a polypeptide” includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

The term “target site” means a location in a polypeptide which can be a single amino acid and/or is a part of, a structural and/or functional motif, e.g., a binding site, a dimerization domain, or a catalytic active site. Target sites may be useful for direct or indirect interaction with an agent, such as a therapeutic agent.

The term “molecular difference” includes any structural and/or functional difference. Methods to detect such differences, as well as examples of such differences, are described herein.

A “functional effect” is a term well known in the art, and means any effect which is exhibited on any level of activity, whether direct or indirect.

The term “ease of harvest” refers to plant characteristics or features that facilitate manual or automated collection of structures or portions (e.g., fruit, leaves, roots) for consumption or other commercial processing.

The term “yield” refers to the amount of plant or animal tissue or material that is available for use by humans for food, therapeutic, veterinary or other markets. Yield can be quantified by a number of direct measures. For example, rice yield can be quantified through direct measures of: yield per acre, grain weight (1000-seed weight, hulled and dehulled seed weight), grain width, grain length, panicle number, seeds per panicle, filled seeds per panicle, weight of filled seeds, % of seed set, panicles per plant, total milled weight, whole milled weight, biomass, and others. Corn yield can be quantified through direct measures such as yield per acre, number of ears, number of kernels per ear, total weight of kernels, and others. Yield can also be quantified by a number of indirect measures, for example, lodging, plant vigor, and measures of yield of grain of acceptable quality, such as, for example, rice yield can be quantified through grain quality measures such as ASV, amylose content, and chalk. Corn yield can be quantified through indirect measures such as amount of starch per kernel, lodging, and others. Plant yield can be measured by total plant biomass, plant growth rate, and others.

The term “yield gene” refers to a gene that has a significant impact on yield or yield-related traits, for example, as quantified by the direct and indirect measures described above.

The term “enhanced economic productivity” refers to the ability to modulate a commercially or aesthetically relevant trait so as to improve desired features. Increased yield and enhanced stress resistance are two examples of enhanced economic productivity.

For the purposes of this invention, the source of the nucleic acid from the domesticated plant or its ancestor or family member, or any other plant, can be any suitable source, e.g., genomic sequences or cDNA sequences. Preferably, cDNA sequences are compared. Protein-coding sequences can be obtained from available private, public and/or commercial databases such as those described herein. These databases serve as repositories of the molecular sequence data generated by ongoing research efforts. Alternatively, protein-coding sequences may be obtained from, for example, sequencing of cDNA reverse transcribed from mRNA expressed in cells, or after PCR amplification, according to methods well known in the art. Alternatively, genomic sequences may be used for sequence comparison. Genomic sequences can be obtained from available public, private and/or commercial databases or from sequencing of genomic DNA libraries or from genomic DNA, after PCR.

In some embodiments, the cDNA is prepared from mRNA obtained from a tissue at a determined developmental stage, or a tissue obtained after the organism has been subjected to certain environmental conditions. cDNA libraries used for the sequence comparison of the present invention can be constructed using conventional cDNA library construction techniques that are explained fully in the literature of the art. Total mRNAs are used as templates to reverse-transcribe cDNAs. Transcribed cDNAs are subcloned into appropriate vectors to establish a cDNA library. The established cDNA library can be maximized for full-length cDNA contents, although less than full-length cDNAs may be used. Furthermore, the sequence frequency can be normalized according to, for example, Bonaldo et al. (1996) Genome Research 6:791-806. cDNA clones randomly selected from the constructed cDNA library can be sequenced using standard automated sequencing techniques. Preferably, full-length cDNA clones are used for sequencing. Either the entire or a large portion of cDNA clones from a cDNA library may be sequenced, although it is also possible to practice some embodiments of the invention by sequencing as little as a single cDNA, or several cDNA clones.

In one preferred embodiment of the present invention, cDNA clones to be sequenced can be pre-selected according to their expression specificity. In order to select cDNAs corresponding to active genes that are specifically expressed, the cDNAs can be subject to subtraction hybridization using mRNAs obtained from other organs, tissues or cells of the same organism. Under certain hybridization conditions with appropriate stringency and concentration, those cDNAs that hybridize with non-tissue specific mRNAs and thus likely represent “housekeeping” genes will be excluded from the cDNA pool. Accordingly, remaining cDNAs to be sequenced are more likely to be associated with tissue-specific functions. For the purpose of subtraction hybridization, non-tissue-specific mRNAs can be obtained from one tissue, or preferably from a combination of different tissues and cells. The amount of non-tissue-specific mRNAs is maximized to saturate the tissue-specific cDNAs.

Alternatively, information from online databases can be used to select or give priority to cDNAs that are more likely to be associated with specific functions. For example, the ancestral cDNA candidates for sequencing can be selected by PCR using primers designed from candidate domesticated organism cDNA sequences. Candidate domesticated organism cDNA sequences are, for example, those that are only found in a specific portion of a plant, or that correspond to genes likely to be important in the specific function. Such specific cDNA sequences may be obtained by searching online sequence databases in which information with respect to the expression profile and/or biological activity for cDNA sequences may be specified.

Sequences of ancestral homologue(s) to a known domesticated organism's gene may be obtained using methods standard in the art, such as PCR methods (using, for example, GeneAmp PCR System 9700 thermocyclers (Applied Biosystems, Inc.)). For example, ancestral cDNA candidates for sequencing can be selected by PCR using primers designed from candidate domesticated organism cDNA sequences. For PCR, primers may be made from the domesticated organism's sequences using standard methods in the art, including publicly available primer design programs such as PRIMER® (Whitehead Institute). The ancestral sequence amplified may then be sequenced using standard methods and equipment in the art, such as automated sequencers (Applied Biosystems, Inc.). Likewise, ancestor or family members gene mimics can be used to obtain corresponding genes in domesticated organisms.

In a preferred embodiment, the methods described herein can be applied to identify the genes that control traits of interest in agriculturally important domesticated plants. Humans have bred domesticated plants for several thousand years without knowledge of the genes that control these traits. Knowledge of the specific genetic mechanisms involved would allow much more rapid and direct intervention at the molecular level to create plants with desirable or enhanced traits.

Humans, through artificial selection, have provided intense selection pressures on crop plants. This pressure is reflected in evolutionarily significant changes between homologous genes of domesticated organisms and their wild ancestor or family members. It has been found that only a few genes, e.g., 10-15 per species, control traits of commercial interest in domesticated crop plants. These few genes have been exceedingly difficult to identify through standard methods of plant molecular biology. The K_{A}/K_{S} and related analyses described herein can identify the genes controlling traits of interest.

In particular, K_{A}/K_{S} type analysis has identified the EG82013 and EG81345 genes as genes that have been positively evolutionarily selected during the course of domestication of rice.

For any crop plant of interest, cDNA libraries can be constructed from the domesticated species or subspecies and its wild ancestor or family member. As is described in U.S. Ser. No. 09/240,915, filed Jan. 29, 1999, (now U.S. Pat. No. 6,228,586), the cDNA libraries of each are “BLASTed” against each other to identify homologous nucleic acids. Alternatively, the skilled artisan can access commercially and/or publicly available genomic or cDNA databases rather than constructing cDNA libraries. All patents and patent applications referenced herein are incorporated by reference into the present document in their entireties

Next, a K_{A}/K_{S} or related analysis may be conducted to identify selected genes that have rapidly evolved under selective pressure. These genes are then evaluated using standard molecular and transgenic plant methods to determine if they play a role in the traits of commercial or aesthetic interest. Using the methods of the invention, the inventors have identified nucleic acids and polypeptides corresponding to genes EG81345 or EG82013, which are yield genes. The genes of interest can be manipulated by, e.g., random or site-directed mutagenesis, to develop new, improved varieties, subspecies, strains or cultivars.

Generally, in one embodiment of the present invention, nucleotide sequences are obtained from a domesticated organism and a wild ancestor or family member. The domesticated organism's and ancestor or family member's nucleotide sequences are compared to one another to identify sequences that are homologous. The homologous sequences are analyzed to identify those that have nucleic acid sequence differences between the domesticated organism and ancestor or family member. Then molecular evolution analysis is conducted to evaluate quantitatively and qualitatively the evolutionary significance of the differences. For genes that have been positively selected, outgroup analysis can be done to identify those genes that have been positively selected in the domesticated organism (or in the ancestor or family member). Next, the sequence is characterized in terms of molecular/genetic identity and biological function. Finally, the information can be used to identify agents that can modulate the biological function of the polypeptide encoded by the gene.

The general methods of the invention entail comparing protein-coding nucleotide sequences of ancestral and domesticated organisms. Bioinformatics is applied to the comparison and sequences are selected that contain a nucleotide change or changes that is/are evolutionarily significant change(s). The invention enables the identification of genes that have evolved to confer some evolutionary advantage and the identification of the specific evolved changes. For example, the domesticated organism may be Oryza sativa and the wild ancestor or family member Oryza rufipogon. In the case of the present invention, protein-coding nucleotide sequences were obtained from plant clones by standard sequencing techniques.

Protein-coding sequences of a domesticated organism and its ancestor or family member are compared to identify homologous sequences. Any appropriate mechanism for completing this comparison is contemplated by this invention.

The following terms are used to describe the sequence relationships between two or more nucleic acids or nucleic acids: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence. (b) As used herein, “comparison window” includes reference to a contiguous and specified segment of a nucleic acid sequence, wherein the nucleic acid sequence may be compared to a reference sequence and wherein the portion of the nucleic acid sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the nucleic acid sequence, a gap penalty is typically introduced and is subtracted from the number of matches. Methods of alignment of sequences for comparison are well-known in the art.

Alignment may be performed manually or by software (examples of suitable alignment programs are known in the art). Preferably, protein-coding sequences from an ancestor/family member or a family member are compared to the domesticated species sequences via database searches, e.g., BLAST searches. The high scoring “hits,” i.e., sequences that show a significant similarity after BLAST analysis, will be retrieved and analyzed. Sequences showing a significant similarity can be those having at least about 60%, at least about 75%, at least about 80%, at least about 85%, or at least about 90% sequence identity. Preferably, sequences showing greater than about 80% identity are further analyzed. The homologous sequences identified via database searching can be aligned in their entirety using sequence alignment methods and programs that are known and available in the art, such as the commonly used simple alignment program CLUSTAL V by Higgins et al. (1992) CABIOS 8:189-191.

In other embodiments, alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, PASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Chapter Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (B) of 10, a cutoff of 100, M=5, N=−4, and a comparison of both strands. For amino acid sequences, the BLASTP program uses as defaults a wordlength (W) of 3, an expectation (B) of 10, and the BLOSUM62 scoring matrix (see Henikoff & Henikoff (1989) Proc. Natl. Acad. Sci, USA 89:10915).

In addition to calculating percent sequence identity, the BLAST algorithm also performs a statistical analysis of the similarity between two sequences (see, e.g., Karlin & Altschul, Proc. Nalt. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Clayerie and States, Comput. Chem., 17:191-201 (1993)) low-complexity filters can be employed alone or in combination. (c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g. as implemented in the program PC/GENE (Intelligenetics, Mountain View, Calif. USA). (d) As used herein, “percentage of sequence identity” means the value determined by comparing two optimally aligned sequences over a comparison window, wherein the portion of the nucleic acid sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. (e) (I) The term “substantial identity” of nucleic acid sequences means that a nucleic acid comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, and most preferably at least 80%.

In one embodiment, for the methods of the invention, the invention provides the following sequences: SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30 SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:58, SEQ ID NO:59, and SEQ ID 60. The invention also provides isolated nucleic acids comprising at least 20 contiguous nucleotides of a nucleic acid provided above, as well as nucleic acids having at least about 50%, at least about 55%, at least about 60%, at least about 65%, at least about 70%, at least about 75%, at least about 80%, at least about 85%, at least about 90%, at least about 95%, to a foregoing nucleic acid and is a marker for yield or a yield gene in a plant, and complements of any of the foregoing nucleic acids. The 20 contiguous nucleotides can be selected from any portion of the nucleic acid sequence.

In one embodiment, the nucleic acid sequence comprises a primer set that when used to genotype a portion of a plant's nucleic acid sequence, creates an amplicon which comprises at least one nucleotide difference which identifies a particular allele, homolog, or ortholog of EG81345 or EG82013. Preferably, the at least one nucleotide difference is linked to a difference in yield. More particularly, in this embodiment, primers are designed to areas adjacent to the targeted amplicon in a EG82013 or EG81345 nucleic acid (such as, for example, an amplicon containing at least one nucleotide change compared to a reference nucleic acid, or one that is identical to a reference nucleic acid, depending on the application), areas distal to it, or areas that overlap it and additional amplicons are produced. Genotyping, in one embodiment, can be accomplished by DNA sequencing of each amplicon. If any nucleotides differ in amplicons from one plant line (inbred or hybrid) compared with an orthologous amplicon from another, statistical analysis is conducted to determine any association with yield of such nucleotide differences. Target amplicons can vary in length. The most useful amplicon length for genotyping is determined empirically. Target amplicons may be designed anywhere throughout the EG82013 or EG81345 chromosomal locus; such amplicons can be adjacent to the target amplicon used in this example, or they may overlap the target amplicon. Alternatively, suitable amplicons may be separated from our target amplicon; they may lie within the EG82013 or EG81345 chromosomal locus, but at some distance either 5′ or 3′ from the target amplicon.

In one embodiment, the at least 20 contiguous nucleic acids of the invention include nucleic acids which include primers and/or primers designed to generate amplicons incorporating one or more of the polymorphisms described above. In another embodiment, the at least 20 contiguous nucleic acids of the invention include nucleic acids which have at least 60% identity to a named EG82013 or EG81324 sequence, at least 65% identity to named EG82013 or EG81324 sequence, at least 70% identity to a named EG82013 or EG81324 sequence, at least 75% identity to a named EG82013 or EG81324 sequence, at least 80% identity to a named EG82013 or EG81324 sequence, at least 85% identity to a named EG82013 or EG81324 sequence, at least 90% identity to a named EG82013 or EG81324 sequence, or at least 95% identity to a named EG82013 or EG81324 sequence.

In another embodiment, the nucleic acid identified by the methods of the invention include nucleotides that are at least 60% identical to at least a 20 contiguous nucleic acid portion to a named EG82013 or EG81324 sequence, nucleotides that are at least 65% identical to at least a 20 nucleic acid portion to a named EG82013 or EG81324 sequence, nucleotides that are at least 70% identical to at least a 20 nucleic acid portion to a named EG82013 or EG81324 sequence, nucleotides that are at least 75% identical to at least a 20 nucleic acid portion to a named EG82013 or EG81324 sequence, nucleotides that are at least 80% identical to at least a 20 nucleic acid portion to a named EG82013 or EG81324 sequence, nucleotides that are at least 85% identical to at least a 20 nucleic acid portion to a named EG82013 or EG81324 sequence, nucleotides that are at least 90% identical to at least a 20 nucleic acid portion to a named EG82013 or EG81324 sequence, or nucleotides that are at least 95% identical to at least a 20 nucleic acid portion to a named EG82013 or EG81324 sequence.

In one embodiment, the present invention includes a method for identifying an EG81345 homolog nucleic acid sequence or an EG81345 allele in a plant. This method includes the following steps. In one step, at least a portion of the plant nucleic acid sequence is compared with at least one nucleic acid. This nucleic acid can be any of the following: an isolated nucleic acid comprising at least 20 contiguous nucleotides of a nucleic acid selected from the group consisting of, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:59, and SEQ ID NO:60; a nucleic acid having at least about 80% sequence identity to a foregoing nucleic acid and is a marker for yield or a yield gene in a plant; and the complement of any of the foregoing nucleic acids. The method also includes identifying at least one nucleic acid sequence that is identical to any of the foregoing nucleic acids in the plant. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

The present invention also includes the steps of comparing at least a portion of the plant polypeptide sequence with at least one EG81345 polypeptide encoded by any of the following: an isolated nucleic acid comprising at least 20 contiguous nucleotides of a nucleic acid selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:59, and SEQ ID NO:60; a nucleic acid having at least about 80% sequence identity to a foregoing nucleic acid and is a marker for yield or a yield gene in a plant; and the complement of any of the foregoing nucleic acids. The method also includes identifying at least one polypeptide sequence that is identical to any of the foregoing polypeptides in the plant. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

In one embodiment, the present invention includes a method for identifying an EG82013 homolog nucleic acid sequence or an EG82013 allele in a plant. This method includes the following steps. In one step, at least a portion of the plant nucleic acid sequence is compared with at least one nucleic acid. This nucleic acid can by any of the following: an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:58; and a nucleic acid having at least about 80% sequence identity to a foregoing nucleic acid and is a marker for yield or a yield gene in a plant; and the complement of any of the foregoing nucleic acids. The method also includes identifying at least one nucleic acid sequence that is identical to any of the foregoing nucleic acids in the plant. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

The present invention also includes the steps of comparing at least a portion of the plant polypeptide sequence with at least one EG82013 polypeptide encoded by any of the following: an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:58; a nucleic acid having at least about 80% sequence identity to a foregoing nucleic acid and is a marker for yield or a yield gene in a plant; and the complement of any of the foregoing nucleic acids. The method also includes identifying at least one polypeptide sequence that is identical to any of the foregoing polypeptides in the plant. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

Several embodiments of the foregoing method are provided. Briefly, the inventors herein have provided the novel insight that EG82013 and EG81345 are yield related genes and/or are markers for yield. The inventors have also shown that EG80312 and EG81345 are yield controlling genes. As such, the present invention provides methods to identify homologous and/or othologous genes, as well as alleles of, EG82013 and EG81345 within a particular species of plant, between closely related species of plant, such as, for example, and ancestor plant and a domestic plant, within a particular family of plants, such as, for example, the Grass family, or less closely related plants. The homologous and/or orthologous genes, and alleles can be determined in a number of plant species, most particularly, those plant species which are domesticated, as described elsewhere herein. Any nucleotide sequence (and/or polypeptide sequence) corresponding to any region of EG82013 and EG81345, or nucleotide sequences (and/or polypeptide sequences) that have significant sequence identity to a region of EG82013 and EG81345, are useful in the invention.

As an example of the method to obtain orthologous sequences in the same species or closely related species, nucleotide sequences corresponding to EG82013 or EG81345 in O. rufipogon or O. sativa can be used as query sequences in a search of O. sativa, O. rufipogon or any other members of the genus Oryza, such as O. nivara. ESTs in GenBank to identify orthologous sequences, searching, for example, within GenBank or any other repository of sequence, or, for example, within a library generated by a skilled practitioner. As another example, nucleotide sequences corresponding to EG82013 or EG81345 obtained from O. sativa, O. rufipogon or any other members of the genus Oryza, such as O. nivara can be used as query sequences in a search of O. sativa, O. rufipogon or any other members of the genus Oryza, such as O. nivara ESTs in GenBank to identify orthologous sequences. A homolog may be understood as a gene related to a second gene by descent from a common ancestral DNA sequence. The term ortholog relates to genes in different species that evolved from a common ancestral gene by speciation.

Alternatively, nucleotide sequences corresponding to EG82013 or EG81345 in O. sativa, O. rufipogon or any other members of the genus Oryza, such as O. nivara can be used as query sequences in a search of genes corresponding to EG82013 or EG81345 in other species, in particular, commercially important species, such as corn, wheat, sorghum, barley, among others, as described elsewhere herein.

It should be noted that a complete protein-coding nucleotide sequence is not required. Indeed, partial cDNA sequences may be compared, such as, for example, sequences derived from ESTs. Once sequences of interest are identified by the methods described below, further cloning and/or bioinformatics methods can be used to obtain the entire coding sequence for the gene or protein of interest. The sequencing and homology comparison of protein-coding sequences between the domesticated organism and its ancestor/family member or a family member may be performed simultaneously by using sequencing chip technology. See, for example, Rava et al. U.S. Pat. No. 5,545,531.

The aligned protein-coding sequences of, for example, domesticated organism and ancestor/family member or a family member are analyzed to identify nucleotide sequence differences and/or peptide sequence differences at particular sites. Again, any suitable method for achieving this analysis is contemplated by this invention. If there are no nucleotide sequence differences, the ancestor/family member or a family member protein coding sequence is not usually further analyzed. The detected sequence changes are generally, and preferably, initially checked for accuracy. Preferably, the initial checking comprises performing one or more of the following steps, any and all of which are known in the art: (a) finding the points where there are changes between the ancestral and domesticated organism sequences; (b) checking the sequence fluorogram (chromatogram) to determine if the bases that appear unique to the ancestor or family member or domesticated organism correspond to strong, clear signals specific for the called base; (c) checking the domesticated organism hits to see if there is more than one domesticated organism sequence that corresponds to a sequence change.

Multiple domesticated organism sequence entries for the same gene that have the same nucleotide at a position where there is a different nucleotide in an ancestor or family member sequence provides independent support that the domesticated sequence is accurate, and that the change is significant. Such changes are examined using database information and the genetic code to determine whether these nucleotide sequence changes result in a change in the amino acid sequence of the encoded protein. As the definition of “nucleotide change” makes clear, the present invention encompasses at least one nucleotide change, either a substitution, a deletion or an insertion, in a protein-coding nucleic acid sequence of a domesticated organism as compared to a corresponding sequence from the ancestor or family member. Preferably, the change is a nucleotide substitution. More preferably, more than one substitution is present in the identified sequence and is subjected to molecular evolution analysis.

This embodiment of the present invention includes methods for identifying allelic variants of the sequences of the present invention. As used herein, “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes in that pair to be followed. A genotype may be defined by use of one or a plurality of markers.

In another embodiment, an EG82013 or EG81345 gene can be an allelic variant that includes a similar but not identical sequence to an EG82013 or EG81345 of the present invention, is a locus (or loci) in the genome whose activity is concerned with the same biochemical or developmental processes, and/or a gene that that occurs at essentially the same locus as the genes including an EG82013 or EG81345 gene of the present invention, but which, due to natural variations caused by, for example, mutation or recombination, has a similar but not identical sequence. Because genomes can undergo rearrangement, the physical arrangement of alleles is not always the same. Allelic variants typically encode polypeptides having similar activity to that of the polypeptide encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given cultivar or strain since the genome is multiploid and/or among a population comprising two or more cultivars or strains. An allele can be defined as a EG81345 or EG82013 nucleic acid sequence having at least one nucleotide change compared to a second EG81345 or EG82013 nucleic acid sequence

As such, the minimal size of a nucleic acid used to encode an EG82013 or EG81345 polypeptide homologue of the present invention is from about 12 to about 18 nucleotides in length. There is no limit, other than a practical limit, on the maximal size of such a nucleic acid in that the nucleic acid can include a portion of a gene, an entire gene, or multiple genes, or portions thereof. Similarly, the minimal size of an EG82013 or EG81345 polypeptide homologue of the present invention is from about 4 to about 6 amino acids in length, with the desired sizes depending on whether a full-length, fusion, multivalent, or functional portions of such polypeptides are desired. In some embodiments, the polypeptide is at least 30 amino acids in length.

As used herein, a EG82013 or EG81345 gene includes all nucleic acid sequences related to a natural EG82013 or EG81345 gene such as regulatory regions that control production of the EG82013 or EG81345 polypeptide encoded by that gene (such as, but not limited to, transcription, translation or post-translation control regions) as well as the coding region itself. In one embodiment, an EG82013 or EG81345 gene includes the EG82013 or EG81345 nucleic acids of the present invention. In another embodiment, an EG82013 or EG81345 gene can be an allelic variant that includes a similar but not identical sequence to the EG82013 or EG81345 nucleic acids of the present invention.

In another use herein, the term “marker for yield” or some other function references the observation that the EG81345 and EG82013 alleles can serve as markers for yield (or some other function) in a plant. The term “yield gene” as used herein references the observation that EG81345 and EG82013 have an impact on yield. In particular, for EG82013, results obtained by the inventor indicated strong, statistically significant associations with yield traits including yield, whole mill, total mill, seed weight, and seed width, as well as agronomic traits lodging and plant height. For EG81345, the inventor has demonstrated strong, statistically significant associations with seed weight, total milling yield, whole weight of milled rice, whole milling yield, and lodging. Because the instant genes were discovered by methods to select evolutionarily significant genes arising during domestication, the allele for each carried by the undomesticated variant of each referenced gene can be a marker for lowered yield relative to the domesticated variant. In addition, among domesticated plant species, and within species, such as among variants, there are allelic differences among EG81345 and EG82013 that are linked to yield and can be correlated with different amounts of yield. Accordingly, the present invention includes within its scope methods to determine which particular allele of these genes a particular plant may contain.

Preferably, the EG82013 or EG81345 nucleic acid sequence is associated with increased yield in a plant. In another embodiment, the EG82013 or EG81345 nucleic acid sequences of the invention modulate yield in a plant. In one embodiment, the EG82013 or EG81345 nucleic acid increases yield in a plant. Methods to determine and quantitate yields are known in the art, and discussed elsewhere in the present specification. For example, increased yield may be increased yield relative to a second plant from a common ancestor, genus or family member plant having a second EG82013 or EG81345 nucleic acid sequence with at least one nucleotide change relative to the EG82013 or EG81345 nucleic acid sequence from the plant.

Accordingly, the present invention also provides isolated nucleic acids comprising nucleic acids of sufficient length and complementarity to a gene of the present invention to use as probes or amplification primers in the detection, quantitation, or isolation of gene transcripts. For example, isolated nucleic acids of the present invention can be used as probes in detecting deficiencies in the level of mRNA in screenings for desired transgenic plants, for detecting mutations in the gene (e.g., substitutions, deletions, or additions), for monitoring upregulation of expression or changes in enzyme activity in screening assays of compounds, for detection of any number of allelic variants (polymorphisms) of the gene, or for use as molecular markers in plant breeding programs.

In particular, the present invention includes a method for identifying one or more alleles of the gene encoding EG81345 in a plant. In particular, this method comprises: assaying a sample of nucleic acids from a plant for the presence of one or more single nucleotide polymorphisms in the plant EG81345 gene, wherein single nucleotide polymorphisms are selected from the group consisting of: SEQ ID NO:51, position 521, C or T; SEQ ID NO:51, position 600, C or T; SEQ ID NO:51, position 644, A or G; SEQ ID NO:51, position 649, G or A; SEQ ID NO:51, position 665, A or G; SEQ ID NO:51, position 807, C or T; SEQ ID NO:51, position 825, T or G; SEQ ID NO:51, position 873, T or C; SEQ ID NO:51, position 924, T or C; and SEQ ID NO:51, position 984, T or C. It has been found that for the ancestral allele SEQ ID NO:49, in the equivalent position to SEQ ID NO:51, encompassing the domestic allele, has T at the equivalent position 71; T at the equivalent position 150; G at the equivalent position 194; A at the equivalent position 199; G at the equivalent position 215; T at the equivalent position 357; G at the equivalent position 375; C at the equivalent position 423; C at the equivalent position 474; C at the equivalent position 534. Conversely, ancestral allele has C at position 71; C at position 150; A at position 194; G at position 199; A at position 215; C at position 357; T at position 375; C at position 423; T at position 474; T at position 534. One of skill in the art will recognize where the corresponding position of each nucleotide change occurs on the domesticated allele, SEQ ID NO:51, by methods such as, for example, lining up the start codons as they occur on SEQ ID NO:51 and SEQ ID NO:49.

In another embodiment, the present invention includes a method for identifying one or more alleles of the gene encoding EG81345 in a plant. In particular, this method includes the following steps. In one step, at least a portion of the plant nucleic acid sequence is compared with at least one nucleic acid which comprises an allele of EG81345. This nucleic acid can be any of the following: an isolated nucleic acid comprising at least 20 contiguous nucleotides of a nucleic acid selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:59, and SEQ ID NO:60; a nucleic acid having at least about 80% sequence identity to a foregoing nucleic acid sequence and is a marker for yield or a yield gene in a plant; and the complement of any of the foregoing nucleic acids. In this method, the nucleic acids comprise, at the equivalent position to SEQ ID NO:51, the following nucleotides: position 521, C or T; SEQ ID NO:51, position 600, C or T; SEQ ID NO:51, position 644, A or G; SEQ ID NO:51, position 649, G or A; SEQ ID NO:51, position 665, A or G; SEQ ID NO:51, position 807, C or T; SEQ ID NO:51, position 825, T or G; SEQ ID NO:51, position 873, T or C; SEQ ID NO:51, position 924, T or C; and SEQ ID NO:51, position 984, T or C.

The present invention also includes a method for identifying one or more alleles of the gene encoding EG82013 in a plant, comprising: assaying a sample of nucleic acids from a plant for the presence of one or more single nucleotide polymorphisms in the plant EG82013 gene, wherein single nucleotide polymorphisms are selected from the group consisting of: SEQ ID NO:2 (or SEQ ID NO:4), position 72, C or T; SEQ ID NO:2 (or SEQ ID NO:4), position 78, C or T; SEQ ID NO:2 (or SEQ ID NO:4), position 132, A or G; SEQ ID NO:2 (or SEQ ID NO:4), position 183, C or T; SEQ ID NO:2 (or SEQ ID NO:4), position 279, A or G; SEQ ID NO:2 (or SEQ ID NO:4), position 306, C or T; SEQ ID NO:2 (or SEQ ID NO:4), position 340, T or C; SEQ ID NO:2 (or SEQ ID NO:4), position 348, G or A; SEQ ID NO:2 (or SEQ ID NO:4), position 414, A or G; SEQ ID NO:2 (or SEQ ID NO:4), position 546, A or G; SEQ ID NO:2 (or SEQ ID NO:4), position 555, T or C; SEQ ID NO:2 (or SEQ ID NO:4), position 690, T to G; SEQ ID NO:2 (or SEQ ID NO:4), position 851, A or G; SEQ SEQ ID NO:2 (or SEQ ID NO:4), position 901, A or G; SEQ ID NO:2 (or SEQ ID NO:4), Position 924, G to A; SEQ ID NO:2 (or SEQ ID NO:4), position 953, C or G; SEQ ID NO:2 (or SEQ ID NO:4), position 1000, G or A; SEQ ID NO:2 (or SEQ ID NO:4), position 1012, A or T; SEQ ID NO:2 (or SEQ ID NO:4), position 1016, T or A; SEQ ID NO:2 (or SEQ ID NO:4), position 1153, A or T; SEQ ID NO:2 (or SEQ ID NO:4), position 1174, G or A; SEQ ID NO:2 (or SEQ ID NO:4), position 1185, T or A; and SEQ ID NO:2 (or SEQ ID NO:4), position 1248, T or A. One of skill in the art will recognize where the corresponding position of each nucleotide change occurs on the domesticated allele and/or related polynucleotides, such as, for example, SEQ ID NO:4, by methods such as, for example, lining up the start codons as they occur on SEQ ID NO:2 and SEQ ID NO:4.

In another embodiment, the present invention includes a method for identifying one or more alleles of the gene encoding EG82013 in a plant. In particular, this method includes the following steps. In one step, at least a portion of the plant nucleic acid sequence is compared with at least one nucleic acid which comprises an allele of EG81345. This nucleic acid can be any of the following: an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:59, and SEQ ID NO:60; a nucleic acid having at least about 80% sequence identity to a foregoing nucleic acid sequence and is a marker for yield or a yield gene in a plant; and the complement of any of the foregoing nucleic acids. In this method, the nucleic acids comprise, at the equivalent position to SEQ ID NO:2 (or SEQ ID NO:4), position 72, C or T; SEQ ID NO:2 (or SEQ ID NO:4), position 78, C or T; SEQ ID NO:2 (or SEQ ID NO:4), position 132, A or G; SEQ ID NO:2 (or SEQ ID NO:4), position 183, C or T; SEQ ID NO:2 (or SEQ ID NO:4), position 279, A or G; SEQ ID NO:2 (or SEQ ID NO:4), position 306, C or T; SEQ ID NO:2 (or SEQ ID NO:4), position 340, T or C; SEQ ID NO:2 (or SEQ ID NO:4), position 348, G or A; SEQ ID NO:2 (or SEQ ID NO:4), position 414, A or G; SEQ ID NO:2 (or SEQ ID NO:4), position 546, A or G; SEQ ID NO:2 (or SEQ ID NO:4), position 555, T or C; SEQ ID NO:2 (or SEQ ID NO:4), position 690, T to G; SEQ ID NO:2 (or SEQ ID NO:4), position 851, A or G; SEQ ID NO:2 (or SEQ ID NO:4), position 901, A or G; SEQ ID NO:2 (or SEQ ID NO:4), Position 924, G to A; SEQ ID NO:2 (or SEQ ID NO:4), position 953, C or G; SEQ ID NO:2 (or SEQ ID NO:4), position 1000, G or A; SEQ ID NO:2 (or SEQ ID NO:4), position 1012, A or T; SEQ ID NO:2 (or SEQ ID NO:4), position 1016, T or A; SEQ ID NO:2 (or SEQ ID NO:4), position 1153, A or T; SEQ ID NO:2 (or SEQ ID NO:4), position 1174, G or A; SEQ ID NO:2 (or SEQ ID NO:4), position 1185, T or A; and SEQ ID NO:2 (or SEQ ID NO:4), position 1248, T or A.

The present inventor has determined that for the domesticated allele, SEQ ID NO:4, at the equivalent position as described above for SEQ ID NO:2, the equivalent position 72 is a T; the equivalent position 78 is a T; the equivalent position 132 is a G; the equivalent position 183 is a T; the equivalent position 279 is a G; the equivalent position 306 is a T; the equivalent position 340 is a C; the equivalent position 348 is an A; the equivalent position 414 is a G; the equivalent position 546 is a G; the equivalent position 555 is a C; the equivalent position 690 is a G; the equivalent position 851 is a G; the equivalent position 901 is a G; the equivalent position 924 is an A; the equivalent position 953 is a G; the equivalent position 1000 is an A; the equivalent position 1012 is a T; the equivalent position 1016 is an A; the equivalent position 1153 is a T; the equivalent position 1174 is an A; the equivalent position 1185 is an A; and the equivalent position 1248 is an A.

Conversely, the present inventor has also determined that for the ancestral allele, SEQ ID NO:2, position 72 is a C, position 78 is a C, position 132 is an A, position 183 is a C, position 279 is an A, position 306 is a C, position 340 is a T, position 348 is a G, position 414 is an A, position 546 is an A, position 555 is a T, position 690 is a T, position 851 is an A, position 901 is an A, position 924 is a G, position 953 is a C, position 1000 is a G, position 1012 is an A, position 1016 is a T, position 1153 is an A, position 1174 is a G, position 1185 is a T; and position 1248 is a T.

Additionally, the present invention further provides isolated nucleic acids comprising nucleic acids encoding one or more polymorphic (allelic) variants of polypeptides/nucleic acids. Polymorphic variants are frequently used to follow segregation of chromosomal regions in, for example, marker assisted selection methods for crop improvement.

The present invention provides a method of genotyping a plant utilizing nucleic acids of the present invention. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., PELEMAN AND VAN DER VOORT, (2003) TRENDS IN PLANT SCIENCE VOL 8(7):330-334 AND HOLLAND (2004) PROCEEDINGS OF THE 4^{TH} INTERNATIONAL CROP SCIENCE Congress 26 Sep.-1 Oct. 2004, Brisbane, Australia. Genotyping provides a measurement of the genetic variation between members of a species. One method of genotyping involves DNA sequencing of the target locus in order to detect single nucleotide polymorphisms (SNP). Single nucleotide polymorphisms (SNP) are the most common type of genetic variation. A SNP is a single base pair mutation at a specific locus, usually consisting of two alleles. Methods for genotyping are known in the art, and include methods such as, for example, hybridization based methods such as dynamic allele-specific hybridization. Briefly, a genomic segment is amplified and attached to a bead through a PCR reaction with a biotinylated primer. In the second step, the amplified product is attached to a streptavidin column and washed with NaOH to remove the unbiotinylated strand. An allele specific oligonucleotide is then added in the presence of a molecule that fluoresces when bound to double-stranded DNA. The intensity is then measured as temperature is increased until the Tm can be determined. An SNP will result in a lower than expected Tm (Howell et al. 1999). Another method relies on molecular beacons. Essentially, SNP detection through molecular beacons makes use of a specifically engineered single-stranded oligonucleotide probe. The oligonucleotide is designed such that there are complementary regions at each end and a probe sequence located in between. This design allows the probe to take on a hairpin, or stem-loop, structure in its natural, isolated state. Attached to one end of the probe is a fluorophore and to the other end a fluorescence quencher. Because of the stem-loop structure of the probe, the fluorophore is in close proximity to the quencher, thus preventing the molecule from emitting any florescence. The molecule is also engineered such that only the probe sequence is complementary to the genomic DNA that will be used in the assay (Abravaya et al. 2003).

Other methods include high density oligonucleotide SNP arrays, where hundreds of thousands of probes are arrayed on a small chip, allowing for a large number of SNPs to be interrogated simultaneously (Rapley & Harbron 2004). Because SNP alleles only differ in one nucleotide and because it is difficult to achieve optimal hybridization conditions for all probes on the array, the target DNA has the potential to hybridize to mismatched probes. This is addressed somewhat by using several redundant probes to interrogate each SNP. Probes are designed to have the SNP site in several different locations as well as containing mismatches to the SNP allele. By comparing the differential amount of hybridization of the target DNA to each of these redundant probes, it is possible to determine specific homozygous and heterozygous alleles (Rapley & Harbron 2004). Although, oligonucleotide microarrays have a comparatively lower specificity and sensitivity, the scale of SNPs that can be interrogated is a major benefit. The Affymetrix Human SNP 5.0 GeneChip performs a genome-wide assay that can genotype over 500,000 human SNPs (Affymetrix 2007).

Other methods include enzyme based methods, such as DNA ligase, DNA polymerase and nucleases for genotyping. Restriction fragment length polymorphism (RFLP) is considered to be the simplest and earliest method to detect SNPs. SNP-RFLP makes use of the many different restriction endonucleases and their high affinity to unique and specific restriction sites. By performing a digestion on a genomic sample and determining fragment lengths through a gel assay it is possible to ascertain whether or not the enzymes cut the expected restriction sites. A failure to cut the genomic sample results in an identifiably larger than expected fragment implying that there is a mutation at the point of the restriction site which is rendering it protected from nuclease activity. The particular method of genotyping in the present invention may employ any number of molecular marker analytic techniques such as, but not limited to, restriction fragment length polymorphisms (RFLPs). RFLPs are the product of allelic differences between DNA restriction fragments caused by nucleotide sequence variability. As is well known to those of skill in the art, RFLPs are typically detected by extraction of genomic DNA and digestion with a restriction enzyme. Generally, the resulting fragments are separated according to size and hybridized with a probe; single copy probes are suitable. Restriction fragments from homologous chromosomes are revealed. Differences in fragment size among alleles represent an RFLP. Thus, the present invention further provides a means to follow segregation of a gene or nucleic acid of the present invention as well as chromosomal sequences genetically linked to these genes or nucleic acids using such techniques as RFLP analysis. Linked chromosomal sequences are within 50 centiMorgans (cM), often within 40 or 30 cM, in some cases within 20 or 10 cM, and in some cases within 5, 3, 2, or 1 cM of a gene of the present invention.

The method of detecting an RFLP comprises the steps of (a) digesting genomic DNA of a plant with a restriction enzyme; (b) hybridizing a nucleic acid probe, under selective hybridization conditions, to a sequence of a nucleic acid of the present of said genomic DNA; (c) detecting therefrom a RFLP. Other methods of differentiating polymorphic (allelic) variants of nucleic acids of the present invention can be had by utilizing molecular marker techniques well known to those of skill in the art including such techniques as: 1) single stranded conformation analysis (SSCP); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6) allele-specific PCR. Other approaches based on the detection of mismatches between the two complementary DNA strands include clamped denaturing gel electrophoresis (CDGE); heteroduplex analysis (HA); and chemical mismatch cleavage (CMC).

Other methods include PCR-based methods, such as Tetra-primer ARMS-PCR, which employs two pairs of primers to amplify two alleles in one PCR reaction. The primers are designed such that the two primer pairs overlap at a SNP location but each match perfectly to only one of the possible SNPs. As a result, if a given allele is present in the PCR reaction, the primer pair specific to that allele will produce product but not to the alternative allele with a different SNP. The two primer pairs are also designed such that their PCR products are of a significantly different length allowing for easily distinguishable bands by gel electrophoresis.

Flap endonuclease (FEN) is an endonuclease that catalyzes structure-specific cleavage. This cleavage is highly sensitive to mismatches and can be used to interrogate SNPs with a high degree of specificity (Olivier 2005).

Another method is by primer extension. Primer extension is a two step process that first involves the hybridization of a probe to the bases immediately upstream of the SNP nucleotide followed by a ‘mini-sequencing’ reaction, in which DNA polymerase extends the hybridized primer by adding a base that is complementary to the SNP nucleotide. This incorporated base is detected and determines the SNP allele (Syvanen 2001). Because, primer extension is based on the highly accurate DNA polymerase enzyme, the method is generally very reliable. Primer extension is able to genotype most SNPs under very similar reaction conditions making it also highly flexible. The primer extension method is used in a number of assay formats. These formats use a wide range of detection techniques that include MALDI-TOF Mass spectrometry and ELISA-like methods (Rapley & Harbron 2004).

Taq DNA polymerase's 5′-nuclease activity is used in the Taqman assay for SNP genotyping. The Taqman assay is performed concurrently with a PCR reaction and the results can be read in real-time as the PCR reaction proceeds (McGuigan & Ralston 2002). The assay requires forward and reverse PCR primers that will amplify a region that includes the SNP polymorphic site. Another method is using DNA ligase. DNA ligase catalyzes the ligation of the 3′ end of a DNA fragment to the 5′ end of a directly adjacent DNA fragment. This mechanism can be used to interrogate a SNP by hybridizing two probes directly over the SNP polymorphic site, whereby ligation can occur if the probes are identical to the target DNA. In the oligonucleotide ligase assay, two probes are designed; an allele-specific probe which hybridizes to the target DNA so that it's 3′ base is situated directly over the SNP nucleotide and a second probe that hybridizes upstream of the SNP polymorphic site providing a 5′ end for the ligation reaction. If the allele-specific probe matches the target DNA, it will fully hybridize to the target DNA and ligation can occur. Ligation does not generally occur in the presence of a mismatched 3′ base. Ligated or unligated products can be detected by gel electrophoresis, MALDI-TOF mass spectrometry or by capillary electrophoresis for large-scale applications (Rapley & Harbron 2004).

In the present invention, the nucleic acid probes employed for molecular marker mapping of plant nuclear genomes selectively hybridize, under selective hybridization conditions, to a gene encoding a nucleic acid of the present invention. In some embodiments, the probes are selected from nucleic acids of the present invention. Typically, these probes are cDNA probes or Pst I genomic clones. The length of the probes is discussed in greater detail, supra, but are typically at least 15 bases in length, and in some cases at least 20, 25, 30, 35, 40, or 50 bases in length. Generally, however, the probes are less than about 1 kilobase in length. In some embodiments, the probes are single copy probes that hybridize to a unique locus in a haploid chromosome complement. Some exemplary restriction enzymes employed in RFLP mapping are EcoRI, EcoRV, and Sst1. As used herein the term “restriction enzyme” includes reference to a composition that recognizes and, alone or in conjunction with another composition, cleaves at a specific nucleotide sequence.

Thus, the present invention further provides a method of genotyping comprising the steps of contacting, under stringent hybridization conditions, a sample suspected of comprising a nucleic acid of the present invention with a nucleic acid probe. Generally, the sample is a plant sample; a sample suspected of comprising a nucleic acid of the present invention (e.g., a gene, mRNA, or EST). The nucleic acid probe selectively hybridizes, under stringent conditions, to a subsequence of a nucleic acid of the present invention comprising a polymorphic marker. Selective hybridization of the nucleic acid probe to the polymorphic marker nucleic acid sequence yields a hybridization complex. Detection of the hybridization complex indicates the presence of that polymorphic marker in the sample. In some embodiments, the nucleic acid probe comprises a nucleic acid of the present invention.

It is apparent to those skilled in the art that polymorphic variants can be identified for EG82013 and EG81345 by sequencing these genes. It is clear to one skilled in the art that additional polymorphic variants or alleles of EG82013 and EG81345 can be identified by sequencing more corn lines and hybrids, more rice lines and hybrids, more sorghum, barley, wheat lines, millet, or sugar cane lines and association tests can be performed to find the alleles of each of these two genes that are associated with the best phenotype for yield traits (such as, for example, for rice: total yield, seed weight, total milling yield, whole weight of milled rice, whole milling yield, or other yield related traits) or quality traits (such as lodging, plant height, and other quality related traits). Association tests with these additional alleles would indicate which alleles are associated with desired phenotypes for specific traits. Prospective parent inbred lines could then be screened for either the presence of the alleles (or portions of the desired alleles that are diagnostic) associated with best performance for a yield trait (such as total yield, grain weight, grain length, grains per plant, etc.) or best performance for a quality trait (such as ASV or chalk, etc.). Alleles associated with the best performance for a yield trait or a quality trait would be the “desired allele” for attaining the desired phenotype.

In preferred embodiments, the present invention provides methods for identifying alleles of EG82013 or EG81345 in a crop species; methods for determining whether a plant contains a preferred allele of EG82013 or EG81345, and methods for screening plants for preferred alleles of EG82013 or EG81345. Alleles of EG82013 and EG81345 include, for example, a nucleic acid comprising any of the following sequences: an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:58, SEQ ID NO:59, and SEQ ID NO:60; a nucleic acid having at least about 80% sequence identity to a foregoing nucleic acid and is a marker for yield or a yield gene in a plant; and the complement of any of the foregoing nucleic acids. Corresponding polypeptides can also be used in a like manner. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

For methods to identify other alleles of EG82013 or EG81345, methods include in one step, using at least a portion of any sequence from the nucleic acid sequences of the present invention to amplify the corresponding EG82013 or EG81345 sequence in one or more plants of a crop species. In another step, these methods include determining the nucleotide sequence of amplified sequences. In another step, these methods include comparing the amplified sequences to nucleic acid sequences of the present invention to identify any alleles of EG82013 or EG81345 in the tested plants of the crop species.

Generally, these methods also include methods for identifying or determining preferred alleles (e.g., alleles that are associated with a desired trait). In one step, using at least a portion of any sequence from the nucleic acid sequences of the present invention to amplify the corresponding EG82013 or EG81345 sequence in at least two plants for which a particular parameter for a trait has been or can be measured. Such a trait includes yield, for example. In another step, these methods include determining the sequence of EG82013 or EG81345 in each plant. In another step, these methods include identifying preferred alleles or nucleic acid sequences of EG82013 or EG81345. Preferred alleles may be identified by genotyping analysis by determining the association of the allele with the desired trait. Examples of such genotyping analysis can be found herein in the Examples.

Generally, these methods also include methods for screening plants for preferred alleles or nucleic acid sequences. Such methods include using at least a portion of a preferred allele (e.g., alleles associated with a desired trait) to amplify the corresponding EG82013 or EG81345 sequence in a plant, and select those plants that contain the desired allele (or nucleic acid sequence). The present invention also provides a method of producing an EG82013 or EG81345 polypeptide comprising: a) providing a cell transfected with a nucleic acid encoding an EG82013 or EG81345 polypeptide positioned for expression in the cell; b) culturing the transfected cell under conditions for expressing the nucleic acid; and c) isolating the EG82013 or EG81345 polypeptide.

The present invention also provides a method of isolating a yield gene from a recombinant plant cell library. The method includes providing a preparation of plant cell DNA or a recombinant plant cell library; contacting the preparation or plant cell library with a detectably-labeled EG82013 or EG81345 conserved oligonucleotide (generated from an EG82013 or EG81345 nucleic acid sequence of the present invention, as described elsewhere herein) under hybridization conditions providing detection of genes having 50% or greater sequence identity; and isolating a yield-gene by its association with the detectable label.

The present invention also provides a method of isolating a yield gene from plant cell DNA. The method includes providing a sample of plant cell DNA; providing a pair of oligonucleotides having sequence homology to a conserved region of an EG82013 or EG81345 gene oligonucleotides (generated from an EG82013 or EG81345 nucleic acid sequence of the present invention, as described elsewhere herein); combining the pair of oligonucleotides with the plant cell DNA sample under conditions suitable for polymerase chain reaction-mediated DNA amplification; and isolating the amplified yield gene or fragment thereof.

The sequences identified by the methods described herein can be used to identify agents that are useful in modulating domesticated organism-unique, enhanced or altered functional capabilities and/or correcting defects in these capabilities using these sequences. These methods employ, for example, screening techniques known in the art, such as in vitro systems, cell-based expression systems and transgenic animals and plants. The approach provided by the present invention not only identifies rapidly evolved genes, but indicates modulations that can be made to the protein that may not be too toxic because they exist in another species.

The present invention also provides a method of detecting a yield-increasing gene or a yield-increasing allelic variant of a gene in a plant cell which includes the following steps. Steps include contacting a EG82013 or EG81345 nucleic acid or a portion thereof at least about 12 nucleotides, at least about 20 nucleotides, in some cases at least about 30 nucleotides in length with a preparation of genomic DNA from the plant cell under hybridization conditions providing detection of nucleic acid molecule sequences having about 50% or greater sequence identity to a EG82013 or EG81345 nucleic acid of the present invention, such as, for example, a nucleic acid comprising at least 20 contiguous nucleotides of nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:58, SEQ ID NO:59, and SEQ ID NO:60; and a nucleic acid having at least about 80% sequence identity to any of the preceding SEQ ID Nos., and the complement of any of the foregoing; and detecting hybridization, whereby a yield-increasing gene may be identified.

The present invention also provides a method of detecting a yield-increasing gene or a specific yield increasing allelic variant of a gene in a plant cell. This method includes contacting the yield increasing genes EG82013 or EG81345 or a portion of any of these genes at least about 12 nucleotides, in some cases at least about 20 nucleotides in length, in some cases at least about 30 nucleotides in length with a preparation of genomic DNA from the plant cell under hybridization conditions providing detection of nucleic acid molecule sequences having about 50% or greater sequence identity to a nucleic acids of the present invention as described elsewhere herein; and detecting hybridization, whereby a yield-increasing gene or a specific yield increasing allelic variant of a gene may be identified.

The sequences identified by the methods described herein can be used to identify agents that are useful in modulating domesticated organism-unique, enhanced or altered functional capabilities and/or correcting defects in these capabilities using these sequences. These methods employ, for example, screening techniques known in the art, such as in vitro systems, cell-based expression systems and transgenic animals and plants. The approach provided by the present invention not only identifies rapidly evolved genes, but indicates modulations that can be made to the protein that may not be too toxic because they exist in another species.

In one embodiment, the present invention includes a method of determining whether a plant has a particular polypeptide or nucleic acid sequence comprising an EG82013 or a EG81345 sequence. This method includes the following steps. One step includes comparing at least about a portion of polypeptide-coding nucleotide sequence of said plant with at least a portion of a nucleic acid sequence of an EG82013 or EG81345 nucleic acid of the present invention, such as, for example, those comprising at least a portion of a nucleic acid selected from the group consisting of (i) a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:58, SEQ ID NO:59, and SEQ ID NO:60; and a polypeptide encoded by one of the above; and (ii) a nucleic acid having at least about 65% sequence identity to a nucleic acid of (i) and which is a marker for yield or a yield gene, and a polypeptide encoded by one of the above. One of the nucleic acids or polypeptides enumerated above can be selected as the particular nucleic acid or polypeptide (i.e., the nucleic acid or polypeptide of interest, for the determination of whether the plant contains that nucleic acid or polypeptide or a related one.) In another step, the method includes identifying whether the plant contains the particular nucleic acid or polypeptide. Preferably, the plant nucleic acid sequence is genomic DNA or cDNA, or polypeptide derived from cDNA. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

Knowing the nucleic acid sequences of certain plant EG82013 or EG81345 nucleic acids of the present invention allows one skilled in the art to, for example, (a) make copies of those nucleic acids, (b) obtain nucleic acids including at least a portion of such nucleic acids (e.g., nucleic acids including full-length genes, full-length coding regions, regulatory control sequences, truncated coding regions), and (c) obtain EG82013 or EG81345 nucleic acids for other plants. Such nucleic acids can be obtained in a variety of ways including screening appropriate expression libraries with antibodies of the present invention; traditional cloning techniques using oligonucleotide probes of the present invention to screen appropriate libraries or DNA; and PCR amplification of appropriate libraries or DNA using oligonucleotide primers of the present invention. Suitable libraries to screen or from which to amplify nucleic acids include libraries such as genomic DNA libraries, BAC libraries, YAC libraries, cDNA libraries prepared from isolated plant tissues, including, but not limited to, stems, reproductive structures/tissues, leaves, roots, and tillers; and libraries constructed from pooled cDNAs from any or all of the tissues listed above. In the case of rice and corn, BAC libraries, available from Clemson University may be used. Similarly, DNA sources to screen or from which to amplify nucleic acids include plant genomic DNA. Techniques to clone and amplify genes are disclosed, for example, in Sambrook et al., ibid. and in Galun & Breiman, TRANSGENIC PLANTS, Imperial College Press, 1997.

The present invention also includes nucleic acids that are oligonucleotides capable of hybridizing, under stringent hybridization conditions, with complementary regions of other, sometimes longer, nucleic acids of the present invention such as those comprising plant EG82013 or EG81345 genes or other plant EG82013 or EG81345 nucleic acids. Oligonucleotides of the present invention can be RNA, DNA, or derivatives of either. The minimal size of such oligonucleotides is the size required to form a stable hybrid between a given oligonucleotide and the complementary sequence on another nucleic acid of the present invention. Minimal size characteristics are disclosed herein. The size of the oligonucleotide must also be sufficient for the use of the oligonucleotide in accordance with the present invention. Oligonucleotides of the present invention can be used in a variety of applications including, but not limited to, as probes to identify additional nucleic acids, as primers to amplify or extend nucleic acids, as targets for expression analysis, as candidates for targeted mutagenesis and/or recovery, or in agricultural applications to alter EG82013 or EG81345 polypeptide production or activity. Such agricultural applications include the use of such oligonucleotides in, for example, antisense-, triplex formation-, ribozyme- and/or RNA drug-based technologies. The present invention, therefore, includes such oligonucleotides and methods to enhance economic productivity in a plant by use of one or more of such technologies.

In another embodiment, the present invention includes a method of determining whether a plant has a particular nucleic acid sequence comprising an EG82013 sequence. This method includes the step of comparing at least about a portion of the nucleic acid sequence of said plant with at least a portion of an EG82013 nucleic acid sequence of the present invention, such as, for example, a nucleic acid comprising a nucleic acid selected from the group consisting of an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:58; a nucleic acid having at least about 80% sequence identity to a nucleic acid in i) and is a marker for yield or a yield gene in a plant; and the complement of any of these aforementioned nucleic acids. One of the nucleic acids enumerated above can be selected as the particular nucleic acid (i.e., the nucleic acid of interest, for the determination of whether the plant contains that nucleic acid or a related one.) In another step, the method includes identifying whether the plant contains the particular nucleic acid. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

As used herein, “at least a portion” of a nucleic acid or polypeptide means a portion having the minimal size characteristics of such sequences, as described above, or any larger fragment of the full length molecule, up to and including the full length molecule. In one embodiment, “at least a portion” refers to an at least 20 contiguous nucleotides from an EG82013 and/or EG81345. In another embodiment, a portion of a nucleic acid may be at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 22 nucleotides, at least about 24 nucleotides, at least about 26 nucleotides, at least about 28 nucleotides, at least about 30 nucleotides, at least about 32 nucleotides, at least about 34 nucleotides, at least about 36 nucleotides, at least about 38 nucleotides, at least about 40 nucleotides, at least about 45 nucleotides, at least about 50 nucleotides, at least about 55 nucleotides, and so on, going up to the full length nucleic acid. Similarly, a portion of a polypeptide may be at least about 4 amino acids, at least about 5 amino acids, at least about 6 amino acids, at least about 7 amino acids, and so on, going up to the full length polypeptide. The length of the portion to be used will depend on the particular application. As discussed above, a portion of a nucleic acid useful as hybridization probe may be as short as 12 nucleotides; in one embodiment, it is 20 nucleotides. A portion of a polypeptide useful as an epitope may be as short as 4 amino acids; in one embodiment, it as at least about 6 amino acids. A portion of a polypeptide that performs the function of the full-length polypeptide would generally be longer than 4 amino acids.

Other plant EG82013 or EG81345 polypeptides of the present invention are polypeptides that include but are not limited to the encoded polypeptides, full-length polypeptides, processed polypeptides, fusion polypeptides and multivalent polypeptides thereof as well as polypeptides that are truncated homologues of polypeptides that include at least portions of the aforementioned SEQ ID NOs.

Preferred plant nucleic acid sequence includes plant sequence that is derived from genomic DNA or derived from the expressed genes of a plant, i.e., is cDNA. Methods to do so are known in the art and are discussed elsewhere in the instant specification.

Preferably, the EG82013 or EG81345 nucleic acid sequence is associated with increased yield in a plant. Methods to determine and quantitate yields are known in the art, and discussed elsewhere in the present specification (see, for example, the definition of the term “yield” at the beginning of the Detailed Description Of the Invention). Most preferably, yield may be quantitated by determining whether yield is increased relative to a second plant from a common ancestor, genus, or family member plant, more preferably the same species, even more preferably the same cultivar, having a second EG82013 or EG81345 nucleic acid sequence with at least one nucleotide change relative to the EG82013 or EG81345 nucleic acid sequence from the plant.

In all embodiments of the present invention, a preferred nucleic acid sequence includes a nucleic acid having at least about 60% sequence identity to a to a EG82013 or EG81345 nucleic acid of the present invention and has substantially the same effect on yield or is a marker for yield or a yield gene. Preferably, a nucleic acid of the present invention will have at least about 65% identity to, at least about 66% identity to, at least about 67% identity to, at least about 68% identity to, at least about 69% identity to, at least about 70% identity to, at least about 71% identity to, at least about 72% identity to, at least about 73% identity to, at least about 74% identity to, at least about 75% identity to, at least about 76% identity to, at least about 77% identity to, at least about 78% identity to, at least about 79% identity to, at least about 80% identity to, at least about 81% identity to, at least about 82% identity to, at least about 83% identity to, at least about 84% identity to, at least about 85% identity to, at least about 86% identity to, at least about 87% identity to, at least about 88% identity to, at least about 89% identity to, at least about 90% identity to, at least about 91% identity to, more preferably at least about at least about 92% identity to, at least about 93% identity to, at least about 94% identity to, at least about 95% identity to, and even more preferably at least about 95.5% identity to, at least about 96% identity to, at least about 96.5% identity to, at least about 97% identity to, at least about 97.5% identity to, at least about 98% identity to, at least about 98.5% identity to, at least about 99% identity to, at least about 99.5% identity to, or are identical to any of a nucleic acid sequence comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55; SEQ ID NO:56, SEQ ID NO:57, SEQ ID NO:58, SEQ ID NO:59, and SEQ ID NO:60 and is a marker for yield or a yield gene in a plant; and the complement of any of the foregoing nucleic acids.

In all embodiments of the present invention, a preferred polypeptide sequence includes a polypeptide having at least about 60% sequence identity to an EG82013 or EG81345 polypeptide of the present invention and has substantially the same effect on yield and/or is a marker for yield. Preferably, a polypeptide of the present invention will have at least about 65% identity to, at least about 66% identity to, at least about 67% identity to, at least about 68% identity to, at least about 69% identity to, at least about 70% identity to, at least about 71% identity to, at least about 72% identity to, at least about 73% identity to, at least about 74% identity to, at least about 75% identity to, at least about 76% identity to, at least about 77% identity to, at least about 78% identity to, at least about 79% identity to, at least about 80% identity to, at least about 81% identity to, at least about 82% identity to, at least about 83% identity to, at least about 84% identity to, at least about 85% identity to, at least about 86% identity to, at least about 87% identity to, at least about 88% identity to, at least about 89% identity to, at least about 90% identity to, at least about 91% identity to, more preferably at least about at least about 92% identity to, at least about 93% identity to, at least about 94% identity to, at least about 95% identity to, and even more preferably at least about 95.5% identity to, at least about 96% identity to, at least about 96.5% identity to, at least about 97% identity to, at least about 97.5% identity to, at least about 98% identity to, at least about 98.5% identity to, at least about 99% identity to, at least about 99.5% identity to, or are identical to any of a polypeptide sequence encoded by a nucleic acid comprising at least 20 contiguous nucleic acids of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55 SEQ ID NO:56, SEQ ID NO:57, SEQ ID NO:58, SEQ ID NO:59, and SEQ ID NO:60, wherein such nucleic acid is a marker for yield or a yield gene in a plant; or any of a polypeptide sequence selected from the group of a 6 contiguous amino acid sequence selected from the group consisting of SEQ ID NO:5, and SEQ ID NO:52.

In all embodiments of the present invention, the domesticated plants of the present invention preferably include Zea mays mays, Oryza sativa, Triticum aestivum, Hordeum vulgare, Saccharum officinarum, Sorghum bicolor, and Pennisetum typhoides. In all embodiments of the present invention, the wild ancestor or family member plants preferably include wild ancestor or family member plants for a domesticated plant selected from the group consisting of Zea mays mays, Oryza sativa, Triticum aestivum, Hordeum vulgare, Saccharum officinarum, Sorghum bicolor, and Pennisetum typhoides. A particularly preferred wild ancestor or family member plant is Oryza rufipogon. Any plant EG82013 or EG81345 polypeptide is a suitable polypeptide of the present invention. Suitable plants also include Pannicum virgatum, Secale cereale, and Arabidopsis thaliana, and/or Agrostis capillaries, Populus tremula, Gossypium hirsutum, and Solanum tuberosum.

Suitable plants from which to isolate EG82013 or EG81345 polypeptides (including isolation of the natural polypeptide or production of the polypeptide by recombinant or synthetic techniques) include maize, wheat, barley, rye, millet, grasses, chickpea, lentil, flax, olive, fig almond, pistachio, walnut, beet, parsnip, citrus fruits, including, but not limited to, orange, lemon, lime, grapefruit, tangerine, minneola, and tangelo, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugarbeet, sunflower, rapeseed, clover, tobacco, carrot, cotton, alfalfa, rice, potato, eggplant, cucumber, Arabidopsis, and woody plants such as coniferous and deciduous trees, with corn, sorghum, sugarcane, grasses, barley, and wheat being especially desirable.

The present invention also provides a method of producing an EG82013 or EG81345 polypeptide. Steps include providing a cell transfected with a nucleic acid encoding an EG82013 or EG81345 polypeptide positioned for expression in the cell; and culturing the transfected cell under conditions for expressing the nucleic acid; and c) isolating the EG82013 or EG81345 polypeptide.

The present invention also provides methods of modifying the frequency of a yield gene or a gene that is a marker for yield in a plant population, and methods for marker assisted breeding or marker assisted selection which includes the following steps. One step includes screening a plurality of plants using an oligonucleotide as a marker to determine the presence or absence of a yield-associated gene in an individual plant, the oligonucleotide consisting of not more than 300 nucleotides and/or at least 20 contiguous nucleotides of a nucleic acid sequence comprising a nucleic acid sequence selected from the group consisting of an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59, and SEQ ID NO:60; a nucleic acid having at least about 80% sequence identity to a foregoing nucleic acid and is a marker for yield or a yield gene in a plant; and the complement of any of the foregoing nucleic acids. Another step includes selecting at least one individual plant for breeding based on the presence or absence of the yield gene; and another step includes breeding at least one plant thus selected to produce a population of plants having a modified frequency of the yield gene. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

In one embodiment, methods for marker assisted breeding include a method of marker assisted breeding of plants for a particular EG81345 nucleic acid sequence. This embodiment includes the following steps. One step includes comparing, for at least one plant, at least a portion of the nucleotide sequence of said plants with a particular EG81345 nucleic acid sequence of the present invention, such as, for example, a nucleic acid comprising a nucleic acid selected from the group consisting of an isolated nucleic acid comprising at least 20 contiguous nucleotides of a nucleic acid selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:59, and SEQ ID NO:60; a nucleic acid having at least about 80% sequence identity to a foregoing nucleic acid and is a marker for yield in a plant; and the complement of any of the foregoing nucleic acids. This method also includes the step of identifying whether the plant comprises the particular nucleic acid sequence; and the step of breeding a plant comprising the particular nucleic acid sequence to produce progeny. Alternatively, the method comprises comparing one of the above-named SEQ ID NOs, or at least a 20 nucleotide portion thereof, or a complement thereof, with at least a portion of the plant nucleic acid sequence and identifying at least one nucleic acid sequence having at least about 80% sequence identity thereto.

The present invention also includes a method of marker assisted breeding of plants for a particular EG81345 polypeptide sequence. This method includes the following steps. Comparing, for at least one plant, at least a portion of the polypeptide sequence of said plant with the particular EG81345 polypeptide sequence. The polypeptide sequence can include a polypeptide encoded by an isolated nucleic acid comprising at least 20 contiguous nucleotides of a nucleic acid selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:59, and SEQ ID NO:60. The polypeptide sequence can also include a nucleic acid having at least about 80% sequence identity to a foregoing nucleic acid and is a marker for yield in a plant. The method also includes the steps of identifying whether the plant comprises the particular polypeptide sequence; and breeding a plant comprising the particular polypeptide sequence to produce progeny.

Methods for marker assisted breeding also include a method of marker assisted breeding of plants for a particular EG82013 nucleic acid sequence. Steps include comparing, for at least one plant, at least a portion of the nucleotide sequence of the plant with the particular nucleic acid sequence selected from the group consisting of an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:58; a nucleic acid having at least about 80% sequence identity to a foregoing nucleic acid and is a marker for yield in a plant; and the complement of any of the foregoing nucleic acids; identifying whether the plant comprises the particular nucleic acid sequence; and breeding a plant comprising the particular nucleic acid sequence to produce progeny.

The present invention also includes a method of marker assisted breeding of plants for a particular EG82013 polypeptide sequence. This method includes the following steps. Comparing, for at least one plant, at least a portion of the polypeptide sequence of said plant with the particular EG82013 polypeptide sequence. The polypeptide sequence can include a polypeptide encoded by an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:58. The polypeptide sequence can also include a nucleic acid having at least about 80% sequence identity to a foregoing nucleic acid and is a marker for yield in a plant. The method also includes the steps of identifying whether the plant comprises the particular polypeptide sequence; and breeding a plant comprising the particular polypeptide sequence to produce progeny.

These marker assisted breeding methods include a method for selecting plants, for example cereals or grasses (including, but not limited to maize, wheat, barley and other members of the Grass family) or legumes (for example, soy beans), having an altered yield comprising obtaining nucleic acid molecules from the plants to be selected, contacting the nucleic acid molecules with one or more probes that selectively hybridize under stringent or highly stringent conditions to a nucleic acid sequence comprising the EG82013 and EG81345 nucleic acids of the present invention; detecting the hybridization of the one or more probes to the nucleic acid sequences wherein the presence of the hybridization indicates the presence of a gene associated with altered yield; and selecting plants on the basis of the presence or absence of such hybridization. In one embodiment, marker-assisted selection is accomplished in rice. In another embodiment, marker assisted selection is accomplished in wheat using one or more probes which selectively hybridize under stringent or highly stringent conditions to sequences comprising the EG82013 and EG81345 nucleic acids of the present invention. In yet another embodiment, marker assisted selection is accomplished in maize or corn using one or more probes which selectively hybridize under stringent or highly stringent conditions to nucleic acids comprising the EG82013 and EG81345 nucleic acids of the present invention. In still another embodiment, marker assisted selection is accomplished in sorghum using one or more probes which selectively hybridize under stringent or highly stringent conditions to sequences comprising the EG82013 and EG81345 nucleic acids of the present invention. In still another embodiment, marker assisted selection is accomplished in barley using one or more probes which selectively hybridize under stringent or highly stringent conditions to sequences comprising the EG82013 and EG81345 nucleic acids of the present invention. In each case marker-assisted selection can be accomplished using a probe or probes to a single sequence or multiple sequences. If multiple sequences are used they can be used simultaneously or sequentially.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and against the markers of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called Genetic Marker Enhanced Selection.

In one embodiment, the present invention includes EG81345 and EG82013 nucleic acids which include an isolated nucleic acid comprising a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59, and SEQ ID NO:60; a nucleic acid having at least about 80% sequence identity to a foregoing nucleic acid and is a marker for yield or a yield gene in a plant. Isolated nucleic acids also includes complements of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59, and SEQ ID NO:60. Preferably, in this embodiment, a nucleic acid will have at least about 65% identity to, at least about 66% identity to, at least about 67% identity to, at least about 68% identity to, at least about 69% identity to, at least about 70% identity to, at least about 71% identity to, at least about 72% identity to, at least about 73% identity to, at least about 74% identity to, at least about 75% identity to, at least about 76% identity to, at least about 77% identity to, at least about 78% identity to, at least about 79% identity to, at least about 80% identity to, at least about 81% identity to, at least about 82% identity to, at least about 83% identity to, at least about 84% identity to, at least about 85% identity to, at least about 86% identity to, at least about 87% identity to, at least about 88% identity to, at least about 89% identity to, at least about 90% identity to, at least about 91% identity to, more preferably at least about at least about 92% identity to, at least about 93% identity to, at least about 94% identity to, at least about 95% identity to, and even more preferably at least about 95.5% identity to, at least about 96% identity to, at least about 96.5% identity to, at least about 97% identity to, at least about 97.5% identity to, at least about 98% identity to, at least about 98.5% identity to, at least about 99% identity to, at least about 99.5% identity to SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, and is a marker for yield or a yield gene in a plant.

One embodiment of the present invention is an isolated plant nucleic acid that hybridizes under stringent hybridization conditions with at least a portion of at least one of the following genes: an EG82013 or EG81345 gene. The identifying characteristics of such genes are heretofore described. A nucleic acid of the present invention can include an isolated natural plant EG82013 or EG81345 gene or a homologue thereof, the latter of which is described in more detail below. A nucleic acid of the present invention can include one or more regulatory regions, full-length or partial coding regions, or combinations thereof. The minimal size of a nucleic acid of the present invention is the minimal size that can form a stable hybrid with one of the aforementioned genes under stringent hybridization conditions. Suitable plants are disclosed above.

In accordance with the present invention, an isolated nucleic acid is a nucleic acid that has been removed from its natural milieu (i.e., that has been subject to human manipulation). As such, “isolated” does not reflect the extent to which the nucleic acid has been purified. An isolated nucleic acid can include DNA, RNA, or derivatives of either DNA or RNA.

An isolated plant EG82013 or EG81345 nucleic acid of the present invention can be obtained from its natural source either as an entire (i.e., complete) gene or a portion thereof capable of forming a stable hybrid with that gene. An isolated plant EG82013 or EG81345 nucleic acid can also be produced using recombinant DNA technology (e.g., polymerase chain reaction (PCR) amplification, cloning) or chemical synthesis. Isolated plant EG82013 or EG81345 nucleic acids include natural nucleic acids and homologues thereof, including, but not limited to, natural allelic variants and modified nucleic acids in which nucleotides have been inserted, deleted, substituted, and/or inverted in such a manner that such modifications do not substantially interfere with the nucleic acid's ability to encode an EG82013 or EG81345 polypeptide of the present invention or to form stable hybrids under stringent conditions with natural gene isolates.

Once the desired DNA has been isolated, it can be sequenced by known methods. It is recognized in the art that such methods are subject to errors, such that multiple sequencing of the same region is routine and is still expected to lead to measurable rates of mistakes in the resulting deduced sequence, particularly in regions having repeated domains, extensive secondary structure, or unusual base compositions, such as regions with high GC base content. When discrepancies arise, resequencing can be done and can employ special methods. Special methods can include altering sequencing conditions by using: different temperatures; different enzymes; proteins which alter the ability of oligonucleotides to form higher order structures; altered nucleotides such as ITP or methylated dGTP; different gel compositions, for example adding formamide; different primers or primers located at different distances from the problem region; or different templates such as single stranded DNAs. Sequencing of mRNA can also be employed.

A plant EG82013 or EG81345 nucleic acid homologue can be produced using a number of methods known to those skilled in the art (see, for example, Sambrook et al., ibid.). For example, nucleic acids can be modified using a variety of techniques including, but not limited to, classic mutagenesis techniques and recombinant DNA techniques, such as site-directed mutagenesis, chemical treatment of a nucleic acid to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acids and combinations thereof. Nucleic acid homologues can be selected from a mixture of modified nucleic acids by screening for the function of the polypeptide encoded by the nucleic acid (e.g., ability to elicit an immune response against at least one epitope of an EG82013 or EG81345 polypeptide, ability to increase yield in a transgenic plant containing an EG82013 or EG81345 gene) and/or by hybridization with an EG82013 or EG81345 gene.

An isolated nucleic acid of the present invention can include a nucleic acid sequence that encodes at least one plant EG82013 or EG81345 polypeptide of the present invention, examples of such polypeptides being disclosed herein. Although the phrase “nucleic acid” primarily refers to the physical nucleic acid and the phrase “nucleic acid sequence” primarily refers to the sequence of nucleotides on the nucleic acid, the two phrases can be used interchangeably, especially with respect to a nucleic acid, or a nucleic acid sequence, being capable of encoding an EG82013 or EG81345 polypeptide. As heretofore disclosed, plant EG82013 or EG81345 polypeptides of the present invention include, but are not limited to, polypeptides having full-length plant EG82013 or EG81345 coding regions, polypeptides having partial plant EG82013 or EG81345 coding regions, fusion polypeptides, multivalent protective polypeptides and combinations thereof.

At least certain nucleic acids of the present invention encode polypeptides that can selectively bind to immune serum derived from an animal that has been immunized with an EG82013 or EG81345 polypeptide from which the nucleic acid was isolated.

A nucleic acid comprising a nucleic acid of the present invention, when expressed in a suitable plant, is capable of increasing the yield of the plant. As will be disclosed in more detail below, such a nucleic acid can be, or encode, an antisense RNA, a molecule capable of triple helix formation, a ribozyme, or other nucleic acid-based compound.

One embodiment of the present invention is a plant EG82013 or EG81345 nucleic acid that hybridizes under stringent hybridization conditions to an EG82013 or EG81345 nucleic acid of the present invention, or to a homologue of such an EG82013 or EG81345 nucleic acid, or to the complement of such a nucleic acid. A nucleic acid complement of any nucleic acid sequence of the present invention refers to the nucleic acid sequence of the nucleic acid that is complementary to (i.e., can form a complete double helix with) the strand for which the sequence is cited. It is to be noted that a double-stranded nucleic acid molecule of the present invention for which a nucleic acid sequence has been determined for one strand that is represented by a SEQ ID NO also comprises a complementary strand having a sequence that is a complement of that SEQ ID NO. As such, nucleic acids of the present invention, which can be either double-stranded or single-stranded, include those nucleic acids that form stable hybrids under stringent hybridization conditions with either a given SEQ ID NO denoted herein and/or with the complement of that SEQ ID NO, which may or may not be denoted herein. Methods to deduce a complementary sequence are known to those skilled in the art. In some embodiments an EG82013 or EG81345 nucleic acid is capable of encoding at least a portion of an EG82013 or EG81345 polypeptide that naturally is present in plants.

In some embodiments, EG82013 or EG81345 nucleic acids of the present invention hybridize under stringent hybridization conditions with at least one of the following nucleic acids: an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59, and SEQ ID NO:60; or to a homologue or complement of such nucleic acid.

In some embodiments, EG82013 or EG81345 nucleic acids of the present invention include the following nucleic acids: an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:59, and SEQ ID NO:60. Preferably, in this embodiment, a nucleic acid will have at least about 65% identity to, at least about 66% identity to, at least about 67% identity to, at least about 68% identity to, at least about 69% identity to, at least about 70% identity to, at least about 71% identity to, at least about 72% identity to, at least about 73% identity to, at least about 74% identity to, at least about 75% identity to, at least about 76% identity to, at least about 77% identity to, at least about 78% identity to, at least about 79% identity to, at least about 80% identity to, at least about 81% identity to, at least about 82% identity to, at least about 83% identity to, at least about 84% identity to, at least about 85% identity to, at least about 86% identity to, at least about 87% identity to, at least about 88% identity to, at least about 89% identity to, at least about 90% identity to, at least about 91% identity to, more preferably at least about at least about 92% identity to, at least about 93% identity to, at least about 94% identity to, at least about 95% identity to, and even more preferably at least about 95.5% identity to, at least about 96% identity to, at least about 96.5% identity to, at least about 97% identity to, at least about 97.5% identity to, at least about 98% identity to, at least about 98.5% identity to, at least about 99% identity to, at least about 99.5% identity to the above-described sequences and is a marker for yield in a plant.

The present invention also includes an isolated polypeptide which comprises (includes) at least a portion of one or more of a polypeptide encoded by a nucleic acid comprising a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:60, and SEQ ID NO:59; a nucleic acid having at least about 80% sequence identity to a foregoing nucleic acid and is a marker for yield or a yield gene in a plant; and the complement of any of the foregoing nucleic acids; and a polypeptide encoded by a nucleic acid having at least about 80% sequence identity to a nucleic acid enumerated above and confers substantially the same yield as a nucleic acid enumerated above. Isolated polypeptides of the present invention also include at least a six amino acid portion of a polypeptide selected from the group consisting of SEQ ID NO:5 and SEQ ID NO:52 and a polypeptide having at least about 80% sequence identity to an at least six amino acid portion of SEQ ID NO:5 or SEQ ID NO:52, and wherein its coding nucleic acid is a marker for yield or a yield gene in a plant; and a nucleic acid having at least about 80% sequence identity to an at least six amino acid portion of SEQ ID NO:5 or SEQ ID NO:52; and confers substantially the same yield as any of the polypeptides enumerated above. Preferably, in this embodiment, a polypeptide encoded by the nucleic acid, or the polypeptide itself, will have at least about 65% identity to, at least about 66% identity to, at least about 67% identity to, at least about 68% identity to, at least about 69% identity to, at least about 70% identity to, at least about 71% identity to, at least about 72% identity to, at least about 73% identity to, at least about 74% identity to, at least about 75% identity to, at least about 76% identity to, at least about 77% identity to, at least about 78% identity to, at least about 79% identity to, at least about 80% identity to, at least about 81% identity to, at least about 82% identity to, at least about 83% identity to, at least about 84% identity to, at least about 85% identity to, at least about 86% identity to, at least about 87% identity to, at least about 88% identity to, at least about 89% identity to, at least about 90% identity to, at least about 91% identity to, more preferably at least about at least about 92% identity to, at least about 93% identity to, at least about 94% identity to, at least about 95% identity to, and even more preferably at least about 95.5% identity to, at least about 96% identity to, at least about 96.5% identity to, at least about 97% identity to, at least about 97.5% identity to, at least about 98% identity to, at least about 98.5% identity to, at least about 99% identity to, at least about 99.5% identity to SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:29, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:58, SEQ ID NO:60, and SEQ ID NO:59 and is a marker for yield or a yield gene in a plant or its coding nucleic acid is a marker for yield or a yield gene in a plant.

The present invention also includes an isolated polypeptide which comprises (includes) at least a portion of one or more of a polypeptide encoded by a nucleic acid comprising a nucleic acid selected from the group consisting of SEQ ID NO:1, SEQ ID NO:2, SEQ ID NO:3, SEQ ID NO:4, SEQ ID NO:6, SEQ ID NO:7, or SEQ ID NO:8, SEQ ID NO:58, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:59, and SEQ ID NO:60; a nucleic acid having at least about 80% sequence identity to a foregoing nucleic acid and is a marker for yield or a yield gene in a plant; and the complement of any of the foregoing nucleic acids; and a polypeptide encoded by a nucleic acid having at least about 80% sequence identity to a nucleic acid enumerated above and confers substantially the same yield as a nucleic acid enumerated above. Isolated polypeptides of the present invention also include a polypeptide selected from the group consisting of SEQ ID NO:5 and SEQ ID NO:52; and a polypeptide having at least about 80% sequence identity to SEQ ID NO:5 and SEQ ID NO:52, and wherein its coding nucleic acid is a marker for yield or a yield gene in a plant; a nucleic acid having at least about 80% sequence identity to SEQ ID NO:5 and SEQ ID NO:52; and confers substantially the same yield as any of the polypeptides enumerated above. Preferably, in this embodiment, a polypeptide encoded by the nucleic acid, or the polypeptide itself, will have at least about 65% identity to, at least about 66% identity to, at least about 67% identity to, at least about 68% identity to, at least about 69% identity to, at least about 70% identity to, at least about 71% identity to, at least about 72% identity to, at least about 73% identity to, at least about 74% identity to, at least about 75% identity to, at least about 76% identity to, at least about 77% identity to, at least about 78% identity to, at least about 79% identity to, at least about 80% identity to, at least about 81% identity to, at least about 82% identity to, at least about 83% identity to, at least about 84% identity to, at least about 85% identity to, at least about 86% identity to, at least about 87% identity to, at least about 88% identity to, at least about 89% identity to, at least about 90% identity to, at least about 91% identity to, more preferably at least about at least about 92% identity to, at least about 93% identity to, at least about 94% identity to, at least about 95% identity to, and even more preferably at least about 95.5% identity to, at least about 96% identity to, at least about 96.5% identity to, at least about 97% identity to, at least about 97.5% identity to, at least about 98% identity to, at least about 98.5% identity to, at least about 99% identity to, at least about 99.5% identity to SEQ ID NO:1, SEQ ID NO:2, SEQ ID NO:3, SEQ ID NO:4, SEQ ID NO:5, SEQ ID NO:6, SEQ ID NO:7, or SEQ ID NO:8, SEQ ID NO:58, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:59, and SEQ ID NO:60, and is a marker for yield or a yield gene in a plant or its coding nucleic acid is a marker for yield in a plant

According to the present invention, an isolated, or biologically pure, polypeptide, is a polypeptide that has been removed from its natural milieu. As such, “isolated” and “biologically pure” do not necessarily reflect the extent to which the polypeptide has been purified. An isolated EG82013 or EG81345 polypeptide of the present invention can be obtained from its natural source, can be produced using recombinant DNA technology or can be produced by chemical synthesis. An EG82013 or EG81345 polypeptide of the present invention may be identified by its ability to perform the function of natural EG82013 or EG81345 in a functional assay. By “natural EG82013 or EG81345 polypeptide,” it is meant the full length EG82013 or EG81345 polypeptide. The phrase “capable of performing the function of a natural EG82013 or EG81345 in a functional assay” means that the polypeptide has at least about 10% of the activity of the natural polypeptide in the functional assay. In other embodiments, the EG82013 or EG81345 polypeptide has at least about 20% of the activity of the natural polypeptide in the functional assay. In other embodiments, the EG82013 or EG81345 polypeptide has at least about 30% of the activity of the natural polypeptide in the functional assay. In other embodiments, the EG82013 or EG81345 polypeptide has at least about 40% of the activity of the natural polypeptide in the functional assay. In other embodiments, the EG82013 or EG81345 polypeptide has at least about 50% of the activity of the natural polypeptide in the functional assay. In other embodiments, the polypeptide has at least about 60% of the activity of the natural polypeptide in the functional assay. In other embodiments, the polypeptide has at least about 70% of the activity of the natural polypeptide in the functional assay. In other embodiments, the polypeptide has at least about 80% of the activity of the natural polypeptide in the functional assay. In other embodiments, the polypeptide has at least about 90% of the activity of the natural polypeptide in the functional assay. Examples of functional assays include antibody-binding assays, or yield-increasing assays, or direct and indirect measures of yield, as detailed elsewhere in this specification.

As used herein, an isolated plant EG82013 or EG81345 polypeptide can be a full-length polypeptide or any homologue of such a polypeptide. Examples of EG82013 or EG81345 homologues include EG82013 or EG81345 polypeptides in which amino acids have been deleted (e.g., a truncated version of the polypeptide, such as a peptide), inserted, inverted, substituted and/or derivatized (e.g., by glycosylation, phosphorylation, acetylation, myristylation, prenylation, palmitoylation, amidation and/or addition of glycerophosphatidyl inositol) such that the homolog has natural EG82013 or EG81345 activity.

In one embodiment, when the homologue is administered to an animal as an immunogen, using techniques known to those skilled in the art, the animal will produce a humoral and/or cellular immune response against at least one epitope of a EG82013 or EG81345 polypeptide. EG82013 or EG81345 homologues can also be selected by their ability to perform the function of EG82013 or EG81345 in a functional assay.

Plant EG82013 or EG81345 polypeptide homologues can be the result of natural allelic variation or natural mutation. EG82013 or EG81345 polypeptide homologues of the present invention can also be produced using techniques known in the art including, but not limited to, direct modifications to the polypeptide or modifications to the gene encoding the polypeptide using, for example, classic or recombinant DNA techniques to effect gene shuffling or random or targeted mutagenesis.

In accordance with the present invention, a mimetope refers to any compound that is able to mimic the ability of an isolated plant EG82013 or EG81345 polypeptide of the present invention to perform the function of EG82013 or EG81345 polypeptide of the present invention in a functional assay. Examples of mimetopes include, but are not limited to, anti-idiotypic antibodies or fragments thereof, that include at least one binding site that mimics one or more epitopes of an isolated polypeptide of the present invention; non-polypeptideaceous immunogenic portions of an isolated polypeptide (e.g., carbohydrate structures); and synthetic or natural organic molecules, including nucleic acids, that have a structure similar to at least one epitope of an isolated polypeptide of the present invention. Such mimetopes can be designed using computer-generated structures of polypeptides of the present invention. Mimetopes can also be obtained by generating random samples of molecules, such as oligonucleotides, peptides or other organic molecules, and screening such samples by affinity chromatography techniques using the corresponding binding partner.

The minimal size of an EG82013 or EG81345 polypeptide homologue of the present invention is a size sufficient to be encoded by a nucleic acid capable of forming a stable hybrid with the complementary sequence of a nucleic acid encoding the corresponding natural polypeptide. As such, the size of the nucleic acid encoding such a polypeptide homologue is dependent on nucleic acid composition and percent homology between the nucleic acid and complementary sequence as well as upon hybridization conditions per se (e.g., temperature, salt concentration, and formamide concentration). It should also be noted that the extent of homology required to form a stable hybrid can vary depending on whether the homologous sequences are interspersed throughout the nucleic acids or are clustered (i.e., localized) in distinct regions on the nucleic acids. The minimal size of such nucleic acids is typically at least about 12 to about 15 nucleotides in length if the nucleic acids are GC-rich and at least about 15 to about 17 bases in length if they are AT-rich. In some embodiments, the nucleic acid is at least 12 bases in length. A plant EG82013 or EG81345 polypeptide of the present invention is a compound that when expressed or modulated in a plant, is capable of increasing the yield of the plant.

One embodiment of the present invention is a fusion polypeptide that includes EG82013 or EG81345 polypeptide-containing domain attached to a fusion segment. Inclusion of a fusion segment as part of an EG82013 or EG81345 polypeptide of the present invention can enhance the polypeptide's stability during production, storage and/or use. Depending on the segment's characteristics, a fusion segment can also act as an immunopotentiator to enhance the immune response mounted by an animal immunized with an EG82013 or EG81345 polypeptide containing such a fusion segment. Furthermore, a fusion segment can function as a tool to simplify purification of an EG82013 or EG81345 polypeptide, such as to enable purification of the resultant fusion polypeptide using affinity chromatography. A suitable fusion segment can be a domain of any size that has the desired function (e.g., imparts increased stability, imparts increased immunogenicity to a polypeptide, and/or simplifies purification of a polypeptide). It is within the scope of the present invention to use one or more fusion segments. Fusion segments can be joined to amino and/or carboxyl termini of the EG82013 or EG81345-containing domain of the polypeptide. Linkages between fusion segments and EG82013 or EG81345-containing domains of fusion polypeptides can be susceptible to cleavage in order to enable straightforward recovery of the EG82013 or EG81345-containing domains of such polypeptides. Fusion polypeptides are produced in some embodiments by culturing a recombinant cell transformed with a fusion nucleic acid that encodes a polypeptide including the fusion segment attached to either the carboxyl and/or amino terminal end of a EG82013 or EG81345-containing domain.

Some fusion segments for use in the present invention include a glutathione binding domain; a metal binding domain, such as a poly-histidine segment capable of binding to a divalent metal ion; an immunoglobulin binding domain, such as Polypeptide A, Polypeptide G, T cell, B cell, Fc receptor or complement polypeptide antibody-binding domains; a sugar binding domain such as a maltose binding domain from a maltose binding polypeptide; and/or a “tag” domain (e.g., at least a portion of β-galactosidase, a strep tag peptide, other domains that can be purified using compounds that bind to the domain, such as monoclonal antibodies). Other fusion segments include metal binding domains, such as a poly-histidine segment; a maltose binding domain; a strep tag peptide.

With regard to EG82013 or EG81345, some recombinant cells are plant cells. By “plant cell” is meant any self-propagating cell bounded by a semi-permeable membrane and containing a plastid. Such a cell also requires a cell wall if further propagation is desired. Plant cell, as used herein includes, without limitation, algae, cyanobacteria, seeds, suspension cultures, embryos, meristematic regions, callus tissue, leaves, roots, shoots, gametophytes, sporophytes, pollen, and microspores. Characteristics of recombinant cells and transgenic plants and suitable methods are described in WO 03/062382, as well as U.S. Pat. No. 6,040,497, both of which are incorporated by reference in their entireties. For example, expression of genes in corn is known in the art and appropriate promoters are known and may be selected by the knowledgeable artesan. For example, plant expression vectors may be constructed using known maize expression vectors, such as those which can be obtained from Rhone Poulenc Agrochimie. Methods to construct the expression constructs and transformation vectors include standard in vitro genetic recombination and manipulation. See, for example, the techniques described in Weissbach and Weissbach, 1988, Methods For Plant Molecular Biology, Academic Press, Chapters 26-28. The transformation vectors of the invention may be developed from any plant transformation vector known in the art including, but are not limited to, the well-known family of Ti plasmids from Agrobacterium and derivatives thereof, including both integrative and binary vectors, and including but not limited to pBIB-KAN, pGA471, pEND4K, pGV38SO, and pMONSOS. Also included are DNA and RNA plant viruses, including but not limited to CaMV, geminiviruses, tobacco mosaic virus, and derivatives engineered therefrom, any of which can effectively serve as vectors to transfer a coding sequence, or functional equivalent thereof, with associated regulatory elements, into plant cells and/or autonomously maintain the transferred sequence. In addition, transposable elements may be utilized in conjunction with any vector to transfer the coding sequence and regulatory sequence into a plant cell.

To aid in the selection of transformants and transfectants, the transformation vectors may preferably be modified to comprise a coding sequence for a reporter gene product or selectable marker. Such a coding sequence for a reporter or selectable marker should preferably be in operative association with the regulatory element coding sequence described supra.

Reporter genes which may be useful in the invention include but are not limited to the ′3-glucuronidase (GUS) gene (Jefferson et al., Proc. Natl. Acad. Sci. USA, 83:8447 (1986)), and the luciferase gene (Ow et al., Science 234:856 (1986)). Coding sequences that encode selectable markers which may be useful in the invention include but are not limited to those sequences that encode gene products conferring resistance to antibiotics, anti-metabolites or herbicides, including but not limited to kanamycin, hygromycin, streptomycin, phosphinothricin, gentamicin, methotrexate, glyphosate and sulfonylurea herbicides, and include but are not limited to coding sequences that encode enzymes such as neomycin phosphotransferase II (NPTII), chloramphenicol acetyltransferase (CAT), and hygromycin phosphotransferase I (HPT, HYG).

A variety of plant expression systems may be utilized to express the coding sequence or its functional equivalent. Particular plant species may be selected from any dicotyledonous, monocotyledonous species, gymnospermous, lower vascular or non-vascular plant, including any cereal crop or other agriculturally important crop. Such plants include, but are not limited to, alfalfa, Arabidopsis, asparagus, wheat, sugarcane, pearl millet, sorghum, barley, cabbage, carrot, celery, corn, cotton, cucumber, flax, lettuce, oil seed rape, pear, peas, petunia, poplar, potato, rice, beet, sunflower, tobacco, tomato, wheat and white clover. Methods by which plants may be transformed or transfected are well-known to those skilled in the art. See, for example, Plant Biotechnology, 1989, Kung & Arntzen, eds., Butterworth Publishers, ch. 1, 2. Examples of transformation methods which may be effectively used in the invention include but are not limited to Agrobacterium-mediated transformation of leaf discs or other plant tissues, microinjection of DNA directly into plant cells, electroporation of DNA into plant cell protoplasts, liposome or spheroplast fusion, microprojectile bombardment, and the transfection of plant cells or tissues with appropriately engineered plant viruses. Plant tissue culture procedures necessary to practice the invention are well-known to those skilled in the art. See, for example, Dixon, 1985, Plant Cell Culture: A Practical Approach, IRL Press. Those tissue culture procedures that may be used effectively to practice the invention include the production and culture of plant protoplasts and cell suspensions, sterile culture propagation of leaf discs or other plant tissues on media containing engineered strains of transforming agents such as, for example, Agrobacterium or plant virus strains and the regeneration of whole transformed plants from protoplasts, cell suspensions and callus tissues. The invention may be practiced by transforming or transfecting a plant or plant cell with a transformation vector containing an expression construct comprising a coding sequence for the sequence and selecting for transformants or transfectants that express the sequence. Transformed or transfected plant cells and tissues may be selected by techniques well-known to those of skill in the art, including but not limited to detecting reporter gene products or selecting based on the presence of one of the selectable markers described supra. The transformed or transfected plant cells or tissues are then grown and whole plants regenerated therefrom. Integration and maintenance of the coding sequence in the plant genome can be confirmed by standard techniques, e.g., by Southern hybridization analysis, PCR analysis, including reverse transcriptase-PCR(RT-PCR) or immunological assays for the expected protein products. Once such a plant transformant or transfectant is identified, a non-limiting embodiment of the invention involves the clonal expansion and use of that transformant or transfectant in the production of a sequence.

Regulatory elements that may be used in the expression constructs include promoters which may be either heterologous or homologous to the plant cell. The promoter may be a plant promoter or a non-plant promoter which is capable of driving high levels transcription of a linked sequence in plant cells and plants. Non-limiting examples of plant promoters that may be used effectively in practicing the invention include cauliflower mosaic virus (CaMV) 19S or 35S, rbcS, the promoter for the chlorophyll a/b binding protein, AdhI, NOS and HMG2, or modifications or derivatives thereof. The promoter may be either constitutive or inducible. For example, and not by way of limitation, an inducible promoter can be a promoter that promotes expression or increased expression of the nucleic acids of the present invention after mechanical gene activation (MGA) of the plant, plant tissue or plant cell. One non-limiting example of such an MGA-inducible plant promoter is MeGA.

The expression constructs can be additionally modified according to methods known to those skilled in the art to enhance or optimize heterologous gene expression in plants and plant cells. Such modifications include but are not limited to mutating DNA regulatory elements to increase promoter strength or to alter the coding sequence itself. Other modifications include deleting intron sequences or excess non-coding sequences from the 5′and/or 3′ ends of the coding sequence in order to minimize sequence- or distance-associated negative effects on expression of proteins, e.g., by minimizing or eliminating message destabilizing sequences.

The expression constructs may be further modified according to methods known to those skilled in the art to add, remove, or otherwise modify peptide signal sequences to alter signal peptide cleavage or to increase or change the targeting of the expressed polypeptides through the plant endomembrane system. For example, but not by way of limitation, the expression construct can be specifically engineered to target the polypeptide for secretion, or vacuolar localization, or retention in the endoplasmic reticulum (ER).

The present invention also includes isolated antibodies capable of selectively binding to at least a portion of an EG82013 or EG81345 polypeptide of the present invention or to a mimetope thereof. Characteristics of recombinant cells and transgenic plants, and suitable methods are described in WO 03/062382.

The present invention also includes plant cells, which comprise heterologous DNA encoding at least a portion of an EG81345 or EG82013 polypeptide. Such polypeptides are capable of altering the yield of a plant. For example, most preferably the polypeptide is capable of increasing the yield of a plant, and less preferably the polypeptide is capable of decreasing the yield of a plant. The plant cells include the polypeptides of the present invention as described elsewhere herein. Additionally, the present invention includes a propagation material of a transgenic plant comprising the above-described transgenic plant cell.

The present invention also includes transgenic plants containing heterologous DNA which encodes an EG81345 or EG82013 polypeptide that is expressed in plant tissue. Such polypeptides are capable of altering the yield of a plant. The transgenic plants include the polypeptides of the present invention as described elsewhere herein.

The present invention also includes an isolated nucleic acid which includes a promoter operably linked to a nucleic acid that encodes at least a portion of an EG81345 or EG82013 polypeptide in plant tissue. Such polypeptides are capable of altering the yield of a plant. The transgenic plants include the polypeptides of the present invention as described elsewhere herein The nucleic acid can be a recombinant nucleic acid, and may include any promoter, including a promoter native to an EG81345 or EG82013 gene.

The present invention also includes a transfected host cell comprising a host cell transfected with a construct comprising a promoter, enhancer or intron nucleic acid from an EG81345 or EG82013 nucleic acid or any combination thereof, operably linked to a nucleic acid encoding a reporter protein. Such constructs are capable of altering the yield of a plant. The transfected host cells comprise the polypeptides of the present invention as described elsewhere herein.

The present invention also includes a recombinant vector, which includes at least a portion of at least one plant EG82013 or EG81345 nucleic acid of the present invention, inserted into any vector capable of delivering the nucleic acid into a host cell. Characteristics of recombinant molecules and suitable methods are described in WO 03/062382. Suitable nucleic acids to include in recombinant vectors of the present invention are as disclosed herein for suitable plant EG82013 or EG81345 nucleic acids per se. Nucleic acids to include in recombinant vectors, and particularly in recombinant molecules, of the present invention include the EG82013 and EG81345 nucleic acids of the present invention.

As used herein, stringent hybridization conditions refer to standard hybridization conditions under which nucleic acids, including oligonucleotides, are used to identify molecules having similar nucleic acid sequences. Such standard conditions are disclosed, for example, in Sambrook et al., MOLECULAR CLONING: A LABORATORY MANUAL, Cold Spring Harbor Labs Press, 1989. Examples of such conditions are provided in the Examples section of the present application.

As used herein, a EG82013 or EG81345 gene from a particular species of plant includes all nucleic acid sequences related to a natural EG82013 or EG81345 gene such as regulatory regions that control production of the EG82013 or EG81345 polypeptide encoded by that gene (such as, but not limited to, transcription, translation or post-translation control regions) as well as the coding region itself.

The following examples are provided to further assist those of ordinary skill in the art. Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### Example 1

### Discovery of EG82013

A cDNA library was prepared from tissues from O. rufipogon mRNA. Random cDNAs were sequenced in a high-throughput manner. ESTs from this sequencing effort were BLASTed against O. sativa DNA sequences in publicly available databases, such as GenBank. Pairwise comparisons using Ka/Ks analysis as described more fully in U.S. Pat. No. 6,274,319 were conducted. One homologous pair, O. rufipogon EST clone number EG82013 and O. sativa in a known database were found to have a Ka/Ks ratio of 3.9 indicating positive selection. The Ka/Ks ratio correlates with the strength of the selection pressure. The theoretical maximum is 4.0. Thus, EG82013 has been under strong selection pressure due to domestication.

EG82013 is located on chromosome 3, GenBank: AC104487 Pb:58670-59166, in a region associated with QTLs for 1000-grain weight, panicle number, grain yield, spikelet number, amylose content, and head rice percentage (see Example 4). It encodes a unique flavin-dependent monooxygenase related to a family of monooxygenases involved in plant growth. Proteins in this family have been reported to affect plant vigor, total plant growth (biomass), and plant growth rate.

The nucleic acid coding sequence corresponding to O. rufipogon clone number EG82013 is nucleic acid sequence SEQ ID NO: 1 and is called an O. rufipogon EG82013 nucleic acid and is also called the ancestral allele of EG82013. SEQ ID NO:1 is a partial mRNA with start codon at nucleotides 51-53. SEQ ID NO:2 is the partial coding sequence.

A homolog of EG82013 was identified in O. sativa, nucleic acid sequence SEQ ID NO:3. SEQ ID NO:3 is the mRNA transcript with start codon from nucleotides 28-30 and stop codon at nucleotides 1387-1389. SEQ ID NO:4 is the predicted CDS. The predicted polypeptide sequence encoded by SEQ ID NO:4 is polypeptide SEQ ID NO:5. SEQ ID NO:6 includes the position of EG82013 (i.e., exon/intron structure) on rice chromosome 3. Genomic sequence is from Gramene database: exons were all thoroughly sequenced (and their positions verified) by Evolutionary Genomics. Intron GT/AG boundaries can be seen in this sequence. The start codon can be seen at nucleotides 73-75. The first exon is from nucleotides 76-103. The next exon is from nucleotides 457 to 600; nucleotides 1269 to 1327; nucleotides 1883-1950; nucleotides 2038-2089; nucleotides 2974-3162; nucleotides 3250-3853; nucleotides 4117-4329; the stop codon is from nucleotides 4330-4332.

A side by side comparison of SEQ ID NO:2 and SEQ ID NO:4, aligned by start codons, showed that in the process of domestication, a number of nucleotide changes have occurred, relative to the domesticated allele, namely, SEQ ID NO:2, position 72, C to T; SEQ ID NO:2, position 78, C to T; SEQ ID NO:1, position 132, A to G; SEQ ID NO:1, position 183, C to T; SEQ ID NO:2, position 279, A to G; SEQ ID NO:2, position 306, C to T; SEQ ID NO:2, position 340, T to C; SEQ ID NO:2, position 348, G to A; SEQ ID NO:2, position 414, A to G; SEQ ID NO:2, position 546, A to G; SEQ ID NO:2, position 555, T to C; SEQ ID NO:2, position 690, T to G; SEQ ID NO:2, position 851, A to G; SEQ ID NO:2, position 901, A to G; SEQ ID NO:2, Position 924, G to A; SEQ ID NO:2, position 953, C to G; SEQ ID NO:2, position 1000, G to A; SEQ ID NO:2, position 1012, A to T; SEQ ID NO:2, position 1016, T to A; SEQ ID NO:2, position 1153, A to T; SEQ ID NO:2, position 1174, G to A; SEQ ID NO:2, position 1185, T to A; and SEQ ID NO:2, position 1248, T to A.

We inferred the biochemical function of the gene encoded by EG82013 from homology searches. The homology searches indicate that EG82013 is a ubiquinone biosynthesis monooxygenase. There are three conserved protein domains: Monooxygenase (monooxygenases include diverse enzymes that utilize FAD; UBiH, (2-polyprenyl-6-methoxyphenol hydroxylases and related FAD-dependent oxidoreductases [These are involved in energy production and conversion, including coenzyme metabolism]; FixC, (these hydrogenases, also known as flavoproteins, are involved in energy production and conversion.

The region of rice chromosome 3 that is syntenic in maize is maize chromosome 1.

### Example 2

### Discovery of EG 81345

Another homologous pair of sequences identified as positively selected as described in Example 1 is O. rufipogon EST clone number 81345 and O. sativa in a known database, which was found to have a Ka/Ks ratio of 1.9. The nucleic acid coding sequence corresponding to O. rufipogon clone number 81345 is nucleic acid sequence SEQ ID NO:48 and is called an O. rufipogon EG81345 nucleic acid and is also referred to as the ancestral allele herein. SEQ ID NO:48 is a partial mRNA; SEQ ID NO:49 is the partial CDS.

The nucleic acid sequence of the homologous O. sativa nucleic acid is nucleic acid sequence SEQ ID NO:50 and is called an O. sativa EG81345 nucleic acid and is also referred to as the derived or domesticated allele in the Examples below and elsewhere in this application. Start codon is present at 95-97 and stop codon is present at 1175-1177. SEQ ID NO:51 is the coding sequence, and the predicted O. sativa polypeptide is polypeptide SEQ ID NO:52. SEQ ID NO:53 includes the position of EG81345 (i.e., exon/intron structure) on rice chromosome 6. The region of rice chromosome 6 that is syntenic in maize is maize chromosome 9. Genomic sequence is from Gramene database: exons were all thoroughly sequenced (and their positions verified) by Evolutionary Genomics. Intron GT/AG boundaries can be seen in this sequence. The start codon can be seen at nucleotides 335-337; the first exon is from nucleotides 338-554; the next exon is from nucleotides 2128-2721; the next exon is from nucleotides 3699 to 4077; and the stop codon is from nucleotides 4078 to 4080.

EG81345 is located on rice chromosome 6 GenBank: AP004806 Bp:151921-152274 in QTLs for % seed set, grain yield, 1000-grain weight, panicle number, spikelet number, amylose content, head rice percentage, and days to maturity (see Example 4). It encodes a unique B-Box-type zinc finger protein with a B-Box-type zinc finger domain and a zinc binding domain, most likely mediating protein-protein interaction. An Arabidopsis ortholog is reported to confer salt tolerance. (See GenBank accession #Q9SYM2.)

A side by side comparison of SEQ ID NO:51 and SEQ ID NO:49, aligned by start codons, showed that in the process of domestication, a number of nucleotide changes have occurred, relative to the domesticated allele, namely, SEQ ID NO:51, position 521, C to T; SEQ ID NO:51, position 600, C to T; SEQ ID NO:51, position 644, A to G; SEQ ID NO:51, position 649, G to A; SEQ ID NO:51, position 665, A to G; SEQ ID NO:51, position 807, C to T; SEQ ID NO:51, position 825, T to G; SEQ ID NO:51, position 873, T to C; SEQ ID NO:51, position 924, T to C; and SEQ ID NO:51, position 984, T to C.

### Example 3

### BLAST to Identify Additional EG82013 and EG81345 Rice Sequences

O. rufipogon and O. sativa EG82013 nucleic acids were used to further BLAST GenBank to identify homologous genes in rice (see Table 1):

TABLE 1

| | | | Sequence | SEQ ID |
|---|---|---|---|---|
| Species | Accession No. | Tissue | Identity | NO: |
| EG82013 | | | | |
| Rice | CK064309 | nearly all plant tissues | 100% | 22 |
| (Oryza sativa) | | i.e., expressed throughout | | |
| | | the plant | | |
| Rice | CI146733 | undisclosed | 100% | 23 |
| (Oryza sativa) | | | | |
| Rice | CA765660 | undisclosed | 99% | 24 |
| (Oryza sativa) | | | | |
| Rice | AK109847 | undisclosed | 100% | 25 |
| (Oryza sativa) | | | | |
| Rice | NM_001058375 | undisclosed | 100% | 26 |
| (Oryza sativa) | | | | |
| EG81345 | | | | |
| Rice | CB651381 | leaf | 99% | 44 |
| (Oryza sativa) | | | | |
| Rice | CB658637 | leaf | 100% | 45 |
| (Oryza sativa) | | | | |
| Rice | AK072231 | undisclosed | 100% | 46 |
| (Oryza sativa) | | | | |
| Rice | NM_001063369 | undisclosed | 100% | 47 |
| (Oryza sativa) | | | | |

### Example 4

### Published QTLs Associated with EG82013 and EG81345

A search of Gramene (www.gramene.org) indicated that EG82013 and EG81345 are associated with the following QTLs in rice:

EG82013:

CQB5 Trait category: Yield Trait: 1000-grain weight

Lu-C-F Shen-L-H Tan-Z-B Xu-Y-B He-P Chen-Y Zhu-L-H, Comparative mapping of QTLs for agronomic traits of rice across environments by using a doubled-haploid population, Theoretical and applied genetics, 94, 1997, pp. 145-150

Lu-C Shen-L Tan-Z Xu-Y He-P Chen-Y Zhu-L Xu-Y-B, Comparative mapping of QTLs for agronomic traits of rice across environments using a doubled haploid population, Theoretical and applied genetics, 93, 1996, pp. 1211-1217

CQAE21 Trait category: Yield Trait: 1000-grain weight

Moncada-P Martinez-C-P Borrero-J Chatel-M Gauch-H Guimaraes-E Tohme-J McCouch-S-R, Quantitative trait loci for yield and yield components in an Oryza sativa X Oryza rufipogon BC2F2 population evaluated in an upland environment, Theoretical and applied genetics, 102(1), 201, pp. 41-42

AQBK048 Trait category: Yield Trait: % seed set

Li-J-Z Zheng-X-W Zhu-L-H He-P Lu-R-L, Identification and interaction analysis of six agronomic trait loci of rice based on a recombinant inbred population, Acta. Bot. Sinica, 41, 1999, pp. 1199-1203

AQEJ084 Trait Category: Vigor Trait: root exudates (synonym: root activity)

Yan-C-J Liang-G-H Chen-F Li-X Tang-S-Z Yi-C-D Tian-S Lu-J-F Gu-M-H, Mapping quantitative trait loci associated with rice grain shape based on an indica/japonica backcross population, Acta genetica Sinica, 30, 2003, pp. 711-716

AQCN006 Trait Category: Vigor Trait: plant height (synonym: seedling height)

Albar-L Lorieux-M Ahmadi-N Rimbault-I Pinel-A Fargette-A-A-S-D Ghesquiere-A, Genetic basis and mapping of the resistance to rice yellow mottle virus. I. QTLs identification and relationship between resistance and plant morphology, Theoretical and applied genetics, 97, 1998, pp. 1145-1154

AQCN007 Trait Category: Vigor Trait: plant height (synonym: seedling height)

Albar-L Lorieux-M Ahmadi-N Rimbault-I Pinel-A Fargette-A-A-S-D Ghesquiere-A, Genetic basis and mapping of the resistance to rice yellow mottle virus. I. QTLs identification and relationship between resistance and plant morphology, Theoretical and applied genetics, 97, 1998, pp. 1145-1154

AQBP009 Trait Category: Quality Trait: grain core percent white

He-P Li-S-G Qian-Q Ma-Y-Q Li-J-Z Wang-W-M Chen-Y Zhu-L-H, Genetic analysis of rice grain quality, Theoretical and applied genetics, 98, 1999, pp. 502-508

EG81345:

AQCF038 Trait category: Yield (Fertility) Trait: spikelet fertility (% seed set)

Zhuang-J-Y Fan-Y-Y Wu-J-L Xia-Y-W Zheng-K-L, Comparison of the detection of QTL for yield traits in different generations of a rice cross using two mapping approaches, Acta genetica Sinica, 28, 2001, pp. 458-464

AQCF040 Trait category: Yield Trait: grain yield

Zhuang-J-Y Fan-Y-Y Wu-J-L Xia-Y-W Zheng-K-L, Comparison of the detection of QTL for yield traits in different generations of a rice cross using two mapping approaches, Acta genetica Sinica, 28, 2001, pp. 458-464

AQCQ017 Trait category: Yield Trait: 1000 grain weight

Yu-S-B Li-J-X Xu-C-G Tan-Y-F Gao-Y-J Li-X-H Zhang-Q Maroof-M-A, Importance of epistasis as the genetic basis of heterosis in an elite rice hybrid, Proceedings of the National Academy of Sciences of the United States of America, 94, 1997, pp. 9226-9231

AQCY026 Trait Category: Yield Trait: panicle number

Hua-J Xing-Y Wu-W Xu-C Sun-X Yu-S Zhang-Q, Single-locus heterotic effects and dominance by dominance interactions can adequately explain the genetic basis of heterosis in an elite rice hybrid, Proceedings of the National Academy of Sciences of the United States of America, 100, 2003, pp. 2574-2579

AQCF021 Trait Category: Yield Trait: grain yield

Zhuang-J-Y Fan-Y-Y Wu-J-L Xia-Y-W Zheng-K-L, Comparison of the detection of QTL for yield traits in different generations of a rice cross using two mapping approaches, Acta genetica Sinica, 28, 2001, pp. 458-464

AQCF037 Trait Category: Yield Trait: spikelet number

Zhuang-J-Y Fan-Y-Y Wu-J-L Xia-Y-W Zheng-K-L, Comparison of the detection of QTL for yield traits in different generations of a rice cross using two mapping approaches, Acta genetica Sinica, 28, 2001, pp. 458-464

AQFU016 Trait Category: Quality Trait: amylose content Aluko-G Martinez-C Tohme-J Castano-C Bergman-C Oard-J-H, QTL mapping of grain quality traits from the interspecific cross Oryza sativa×O. glaberrima, Theoretical and applied genetics, 2004, pp.—

AQFU006 Trait Category: Quality Trait: head rice percentage

Aluko-G Martinez-C Tohme-J Castano-C Bergman-C Oard-J-H, QTL mapping of grain quality traits from the interspecific cross Oryza sativa x O. glaberrima, Theoretical and applied genetics, 2004, pp.—

AQCF009 Trait Category: Yield Trait: spikelet number

Zhuang-J-Y Fan-Y-Y Wu-J-L Xia-Y-W Zheng-K-L, Comparison of the detection of QTL for yield traits in different generations of a rice cross using two mapping approaches, Acta genetica Sinica, 28, 2001, pp. 458-464

### Example 5

### BLAST to Identify ESTs Homologous to EG82013 in Other Genera

O. rufipogon and O. sativa EG82013 nucleic acids were used to further BLAST GenBank to identify homologous genes in other plants. In this way, the following genes were identified in Table 2:

TABLE 2

| | GenBank | | | |
|---|---|---|---|---|
| | Accession | | % | SEQ ID |
| Species | Number | Tissue | Identity | No. |
| Maize (Zea mays) | DT938493 | nearly all plant tissues | 85% | 27 |
| Maize (Zea mays) | DR955187 | nearly all plant tissues | 85% | 28 |
| Maize (Zea mays) | DR831207 | nearly all plant tissues | 84% | 29 |
| Sorghum | BG103656 | rhizome | 84% | 30 |
| (Sorghum propinquum) | | | | |
| Sorghum | BG35665 | mix of ovaries | 84% | 9 |
| (Sorghum bicolor) | | | | |
| Sugarcane | CA279855 | whole seedlings | 88% | 10 |
| (Saccharum officinarum) | | | | |
| Bread wheat | CJ724311 | unknown | 86% | 11 |
| (Triticum aestivum) | | | | |
| Bread wheat | BG904046 | unknown | 86% | 12 |
| (Triticum aestivum) | | | | |
| Bread wheat | CJ648827 | dormant seed | 86% | 13 |
| (Triticum aestivum) | | | | |
| Barley | BQ462082 | unknown | 87% | 14 |
| (Hordeum vulgare) | | | | |
| Colonial Bentgrass | DV856125 | leaf | 90% * | 15 |
| (Agrostis capillaries) | | | | |
| Poplar | BU888909 | petiole | 68% | 16 |
| (Populus tremula) | | | | |
| Poplar | BU827243 | apical shoot | 66% | 17 |
| (Populus tremula × | | | | |
| Populus tremuloides) | | | | |
| Upland Cotton | CO496355 | cotton lint fiber | 67% | 18 |
| (Gossypium hirsutum) | | | | |
| Potato | CN214658 | Callus-derived | 80% | 19 |
| (Solanum tuberosum) | | suspension culture | | |
| Arabidopsis | NM_001035681 | unknown | 66.5% | 20 |
| (Arabidopsis thaliana) | | | | |
| Arabidopsis | CK120474 | inflorescence meristem | 66.5% | 21 |
| (Arabidopsis thaliana) | | | | |

* Only a very small portion of the gene was available

### Example 6

### BLAST to Identify ESTs Homologous to EG81345 in Other Genera

O. rufipogon and O. sativa EG82013 nucleic acids were used to further BLAST GenBank to identify homologous genes in other plants. In this way, the following genes were identified in Table 3:

TABLE 3

| | GenBank | | | |
|---|---|---|---|---|
| | Accession | | % | SEQ |
| Species | Number | Tissue | Identity | ID No. |
| Maize (Zea mays) | DQ245906 | silks, husks, ears, pollen, | 84% | 31 |
| | | shoot tips, leaf, root tips, | | |
| | | whole seed, embryo | | |
| Maize (Zea mays) | EB409030 | silks, husks, ears, pollen, | 85% | 32 |
| | | shoot tips, leaf, root tips, | | |
| | | whole seed, embryo | | |
| Maize (Zea mays) | EE014060 | silks, husks, ears, pollen, | 85% | 33 |
| | | shoot tips, leaf, root tips, | | |
| | | whole seed, embryo | | |
| Sorghum | CX606373 | root | 83% | 34 |
| (Sorghum bicolor) | | | | |
| Sorghum | CN144762 | wounded leaves | 83% | 35 |
| (Sorghum bicolor) | | | | |
| Sugarcane | CA298075 | developing inflorescence | 83% | 36 |
| (Saccharum officinarum) | | and rachis | | |
| Sugarcane | CA176104 | first apical stalk internodes | 83% | 37 |
| (Saccharum officinarum) | | of adult plants | | |
| Bread wheat | BT009289 | unknown | 82% | 38 |
| (Triticum aestivum) | | | | |
| Bread wheat | CK202082 | root | 80% | 39 |
| (Triticum aestivum) | | | | |
| Barley | CV056260 | endosperm | 80% | 40 |
| (Hordeum vulgare) | | | | |
| Switchgrass | DN141282 | crown | 83% | 41 |
| (Panicum virgatum | | | | |
| cultivar Kanlow) | | | | |
| Rye (Secale cereale) | BE495742 | anther | 80% | 42 |
| Arabidopsis | NM_106507 | unknown | <60% | 43 |
| (Arabidopsis thaliana) | | | | |

### Example 7

### Genotyping EG82013 in Rice Lines and Hybrids and Statistical Analysis

EG82013 nucleic acids were PCR amplified from rice lines and hybrids and their nucleic acid sequences were determined. Generally, the higher yielding lines and hybrids were found to have the derived allele of EG82013 and the lower yielding lines and hybrids were found to have the ancestral allele of EG82013. In fact, the average yield was 7,136 lbs/acre for entries with the ancestral allele compared with 8,806 lbs/acre for entries with the derived allele of EG82013. Association with a commercially important traits was determined using statistical software including SAS (SAS Institute. SAS/STAT User's Guide Version 8 (SAS Institute, Cary, N.C., 1999) and TASSEL (Yu, Jainming et al. Nature Genetics 2006 38(2):203-208). These statistical software packages calculate R² values which indicate the percent of the variance in a particular trait that can be explained by a particular gene or locus. Results indicated strong, statistically significant associations with yield traits including yield, whole mill, total mill, seed weight, and seed width, as well as agronomic traits lodging and plant height (see Table 4).

TABLE 4
Association analysis results for EG 82013

| | | Whole | Total | | | | |
|---|---|---|---|---|---|---|---|
| | | Milling | Milling | Seed | Seed | | Plant |
| Trait | Yield | Yield | Yield | Weight | Width | Lodging | Height |
| R² | 27% | 31% | 22% | 28% | 21% | 26% | 21% |
| Value | | | | | | | |

### Example 8

### Genotyping EG81345 in Rice Lines and Hybrids and Statistical Analysis

EG81345 nucleic acids were PCR amplified from rice lines and hybrids and their nucleic acid sequences were determined. Statistical analysis was conducted as described above and indicated strong, statistically significant associations with seed weight, total milling yield, whole weight of milled rice, whole milling yield, and lodging (see Table 5).

TABLE 5
Association analysis results for EG 81345

| | Whole | Total | | | |
|---|---|---|---|---|---|
| | Milling | Milling | Seed | Seed | |
| Trait | Yield | Yield | Weight | Width | Lodging |
| R² Value | 48% | 20% | 31% | 30% | 27% |

### Example 9

### Expression Profile for EG82013

Expression profile, shown in the below table, suggested by analysis of EST counts in GenBank showed that expression of EG82013 was highest in inflorescence, compared to callus, flower, leaf, root, seed, and stem.

| | | |
|---|---|---|
| callus | 0 | 0/15887 |
| flower | 0 | 0/5122 |
| inflorescence | 47 | 2/41731 |
| leaf | 0 | 0/44341 |
| root | 0 | 0/4902 |
| seed | 0 | 0/7737 |
| stem | 0 | 0/2264 |

### Example 10

### Expression Profile for EG81345

Expression profile, shown in the below table (which is an analysis of EST counts in GenBank) showed that expression of EG81345 was highest in the inflorescence, seed and callus, as compared to flower, leaf, root, and stem.

| | | |
|---|---|---|
| callus | 62 | 1/15887 |
| flower | 0 | 0/5122 |
| inflorescence | 23 | 1/41731 |
| leaf | 0 | 0/44341 |
| root | 0 | 0/4902 |
| seed | 129 | 1/7737 |
| stem | 0 | 0/2264 |

### Example 11

### Alleles of EG81345

One hundred and fourteen rice lines and hybrids were genotyped for the EG81345 locus to determine the extent and nature of allelic diversity. We targeted a portion of the EG81345 locus that appeared to contain sufficient nucleotide diversity to allow differentiation of alleles. Primers designated MR07021F (5′⁻³¹ TTC AGT TGG CTG TGT CAT GTG C⁻3′) (SEQ ID NO:54) and MR07022R (5′⁻GGT ACA GGT TTT CAC TTA ACG AAT GAC G⁻3′) (SEQ ID NO:55) were designed using standard techniques to produce an amplicon of 1026 bases for each sample (line or hybrid). Genotyping was accomplished by DNA sequencing the same 835 base pair section from each amplicon. Only two alleles were identified in the 114 rice lines and hybrids.

### Example 12

### Additional Alleles of EG81345

Examination of additional rice germplasm shows that at least a few more alleles are found. Examination at other non-coding regions within the EG81345 locus other than the 835 contiguous base pairs discussed previously, reveals more alleles.

Primers are designed to areas adjacent to the targeted amplicon described above, areas distal to it, or areas that overlap it and additional amplicons are produced. Genotyping is accomplished by DNA sequencing of each amplicon. If any nucleotides differ in amplicons from one line compared with an orthologous amplicon from another, statistical analysis is conducted to determine any association with yield of such nucleotide differences. Target amplicons can vary in length. The most useful amplicon length for genotyping is determined empirically. Target amplicons may be designed anywhere throughout the EG81345 chromosomal locus; such amplicons can be adjacent to the target amplicon used in this example, or they may overlap the target amplicon. Alternatively, suitable amplicons may be separated from our target amplicon; they may lie within the EG81345 chromosomal locus, but at some distance either 5′ or 3′ from our target amplicon.

One of skill in the art can utilize EG81345 sequences provided herein to sequence in a set of rice lines to find markers for the two EG81345 alleles we identified (the O. rufipogon sequence and the O. sativa sequence) and identify new alleles that have different effects on yield.

### Example 13

### Alleles of EG82013

One hundred and fourteen rice lines and hybrids were genotyped for the EG82013 locus to determine the extent and nature of allelic diversity. We targeted a portion of the EG82013 locus that appeared to contain sufficient nucleotide diversity to allow differentiation of alleles. Primers designated MR07033F (5′-CAC CCA TGT GTA GGA CAG AGT AAA CC-3) (SEQ ID NO:7) and MR070008R (5′-5′-CAA CAG AGT ACA TCT TCT GGA AAC CAT CTA GC-3′) (SEQ ID NO:8) were designed using standard techniques to produce an amplicon of 1354 bases for each sample (line or hybrid). Genotyping was accomplished by DNA sequencing the same 636 base pair section from each amplicon. Only two alleles were identified in the 114 rice lines and hybrids.

### Example 14

### Additional Alleles of EG82013

Primers are designed to areas adjacent to the targeted amplicon described above, areas distal to it, or areas that overlap it and additional amplicons are produced. Genotyping is accomplished by DNA sequencing of each amplicon. If any nucleotides differ in amplicons from one line compared with an orthologous amplicon from another, statistical analysis is conducted to determine any association with yield of such nucleotide differences. Target amplicons can vary in length. The most useful amplicon length for genotyping is determined empirically. Target amplicons may be designed anywhere throughout the EG82013 chromosomal locus; such amplicons can be adjacent to the target amplicon used in this example, or they may overlap the target amplicon. Alternatively, suitable amplicons may be separated from our target amplicon; they may lie within the EG82013 chromosomal locus, but at some distance either 5′ or 3′ from our target amplicon.

### Example 15

### Identification of EG82013 and EG81345 in Zea mays mays

Another homologous pair of sequences identified as homologues to EG81345 and EG82013, identified in Example 1 and Example 2, was obtained from Zea mays mays. The nucleic acid mRNA corresponding to EG 82013 in Zea mays mays is nucleic acid sequence SEQ ID NO:58 having a start codon at nucleotides 52-54 and a stop codon at 1618-1620. The nucleic acid mRNA (partial) corresponding to EG81345 in Zea mays mays is nucleic acid sequence SEQ ID NO:59 and SEQ ID NO:60 (both partial mRNA).

What is claimed is:

1-7. (canceled)

8. An isolated EG82013 nucleic acid or polypeptide comprising a nucleic acid or polypeptide selected from the group consisting of:
a) SEQ ID NO:1, SEQ ID NO:2, SEQ ID NO:3, SEQ ID NO:4, SEQ ID NO:6, SEQ ID NO:7, SEQ ID NO:8, and SEQ ID NO:58;
b) a nucleic acid having at least about 80% sequence identity to a nucleic acid in a) and is a yield gene or a marker for yield in a plant;
c) a polypeptide encoded by a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, and SEQ ID NO:58;
d) a polypeptide encoded by a nucleic acid having at least about 80% sequence identity to a nucleic acid in a) and wherein its coding nucleic acid is a yield gene or a marker for yield in a plant;
e) a polypeptide comprising SEQ ID NO:5; and
f) a polypeptide comprising a polypeptide having at least about 80% sequence identity to a polypeptide of d) and wherein its coding nucleic acid is a yield gene or a marker for yield in a plant.

9. (canceled)

10. A plant cell comprising heterologous DNA comprising a nucleic acid or polypeptide of claim 8.

11. A propagation material of a transgenic plant comprising the transgenic plant cell according to claim 10.

12. A transgenic plant comprising heterologous DNA comprising a nucleic acid of claim 8.

13. An isolated nucleic acid which includes a promoter operably linked to a nucleic acid of claim 8.

14. The isolated nucleic acid of claim 13, wherein said nucleic acid is a recombinant nucleic acid.

15. A transfected host cell comprising a host cell transfected with a construct comprising a promoter, enhancer or intron nucleic acid from an EG82013 nucleic acid of claim 8, operably linked to a nucleic acid encoding a reporter protein.

16. A method for identifying an EG81345 or EG82013 homolog nucleic acid sequence or an EG81345 or EG82013 allele in a plant, comprising the steps of:
a) comparing at least a portion of the plant nucleic acid sequence with at least one nucleic acid selected from the group consisting of:
i) an isolated nucleic acid comprising at least 20 contiguous nucleotides of a nucleic acid selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:53, SEQ ID NO:54, SEQ ID NO:55, SEQ ID NO:56, SEQ ID NO:57, SEQ ID NO:59, and SEQ ID NO:60;
ii) an isolated nucleic acid comprising at least 20 contiguous nucleotides of a nucleic acid selected from the group consisting of SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:58;
iii) the complement of a nucleic acid of i) or ii); and
b) identifying at least one nucleic acid sequence having at least about 80% sequence identity to a nucleic acid in i), ii) or iii) and is a yield gene or a marker for yield in a plant.

17-24. (canceled)

25. The method of claim 16, wherein the plant nucleic acid sequence is genomic DNA.

26. The method of claim 16, wherein the plant nucleic acid sequence is cDNA.

27. The method of claim 16, wherein the EG82013 nucleic acid sequence modulates yield in a plant.

28. The method of claim 16, wherein modulated yield is relative to a second plant from the same genus having a second EG82013 nucleic acid sequence with at least one nucleotide change relative to the EG82013 nucleic acid sequence from the plant.

29. The method of claim 16, wherein the plant is selected from the group consisting of Zea mays mays, Oryza sativa, Triticum aestivum, Hordeum vulgare, Saccharum officinarum, Sorghum bicolor, Agrostis capillaries, Populus tremula, Gossypium hirsutum, Solanum tuberosum, and Arabidopsis thaliana.

30. The method of claim 16, wherein the plant is selected from the group consisting of a wild ancestor plant for a domesticated plant selected from the group consisting of Zea mays mays, Oryza sativa, Triticum aestivum, Hordeum vulgare, Saccharum officinarum, Sorghum bicolor, Agrostis capillaries, Populus tremula, Gossypium hirsutum, Solanum tuberosum, and Arabidopsis thaliana.

31-50. (canceled)

51. A method for identifying one or more alleles of the gene encoding EG82013 in a plant, comprising: assaying a sample of nucleic acids from a plant for the presence of one or more single nucleotide polymorphisms in the plant EG82013 gene, wherein single nucleotide polymorphisms are selected from the group consisting of: SEQ ID NO:2, position 72, C or T; SEQ ID NO:2, position 78, C or T; SEQ ID NO:2, position 132, A or G; SEQ ID NO:2, position 183, C or T; SEQ ID NO:2, position 279, A or G; SEQ ID NO:2, position 306, C or T; SEQ ID NO:2, position 340, T or C; SEQ ID NO:2, position 348, G or A; SEQ ID NO:2, position 414, A or G; SEQ ID NO:2, position 546, A or G; SEQ ID NO:2, position 555, T or C; SEQ ID NO:2, position 690, T to G; SEQ ID NO:2, position 851, A or G; SEQ ID NO:2, position 901, A or G; SEQ ID NO:2, Position 924, G to A; SEQ ID NO:2, position 953, C or G; SEQ ID NO:2, position 1000, G or A; SEQ ID NO:2, position 1012, A or T; SEQ ID NO:2, position 1016, T or A; SEQ ID NO:2, position 1153, A or T; SEQ ID NO:2, position 1174, G or A; SEQ ID NO:2, position 1185, T or A; and SEQ ID NO:2, position 1248, T or A.
